(19) United States
(12) Patent Application Publication
(10) Pub. No.: US 2022/0348549 A1
(43) Pub. Date: Nov. 3, 2022

(54) QUINAZOLINE COMPOUNDS FOR THE TREATMENT AND PROPHYLAXIS OF HEPATITIS B VIRUS DISEASE

(71) Applicant: Hoffmann-La Roche Inc., Little Falls, NJ (US)

(72) Inventors: Dongdong CHEN, Shanghai (CN); Song FENG, Shanghai (CN); Chungen LIANG, Shanghai (CN); Chao LI, Shanghai (CN); Zongxing QIU, Shanghai (CN); Xuefei TAN, Pudong, Shanghai (CN); Guolong WU, Shanghai (CN)

(73) Assignee: Hoffmann-La Roche Inc., Little Falls, NJ (US)

(21) Appl. No.: 17/639,110

(22) PCT Filed: Aug. 26, 2020
(86) PCT No.: PCT/EP2020/073780

§ 371 Date: Feb. 28, 2022

(30) Foreign Application Priority Data
Aug. 28, 2019 (CN) PCT/CN2019/102958

(51) Int. Cl. C07D 239/74; C07D 401/04; C07D 401/14; C07D 239/91; A61P 31/20

(52) U.S. Cl. CPC C07D 239/74; C07D 401/04; C07D 401/14; C07D 239/91; A61P 31/20

(57) ABSTRACT

The present invention provides novel compounds having the general formula: wherein R¹ to R⁶ and A are as described herein, compositions including the compounds and methods of using the compounds.

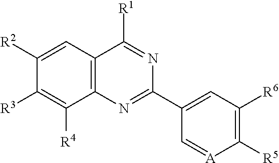

The present invention relates to organic compounds useful for therapy and/or prophylaxis of HBV infection in a mammal, and in particular to HBV cccDNA (covalently closed circular DNA) inhibitors useful for treating HBV infection.

### FIELD OF THE INVENTION

The present invention relates to novel quinazoline derivatives having pharmaceutical activity, their manufacture, pharmaceutical compositions containing them and their potential use as medicaments.

The present invention relates to compounds of formula (I)

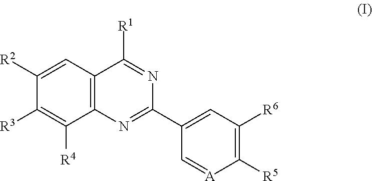

wherein R¹ to R⁶ and A are as described below, or pharmaceutically acceptable salt, or enantiomer, or diastereomer thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are novel compounds of formula (I), their manufacture, medicaments based on a compound in accordance with the invention and their production as well as the use of compounds of formula (I) as HBV cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

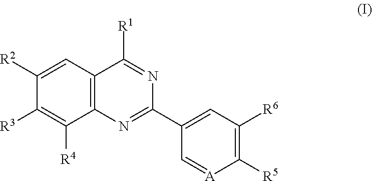

wherein,
A is CH or N;
R¹ is H, (C₁₋₆alkyl)₂aminoC₁₋₆alkyl, C₁₋₆alkoxy, C₁₋₆alkoxyC₁₋₆alkyl, C₁₋₆alkoxycarbonyl, C₁₋₆alkyl, C₁₋₆alkyl-C(O)O—C₁₋₆alkyl-, carbamoyl, carboxy, haloC₁₋₆alkyl, haloC₁₋₆alkylaminocarbonyl, hydroxy, hydroxyC₁₋₆alkyl, hydroxyC₁₋₆alkylaminocarbonyl, morpholinylC₁₋₆alkyl or morpholinylcarbonyl;
R² is H or halogen;
R³ is H, halogen or haloC₁₋₆alkyl;
R⁴ is H or halogen;
R⁵ is H, (C₁₋₆alkoxycarbonyl)phenylC₁₋₆alkoxy, (C₁₋₆alkoxycarbonyl)piperidinyl, (C₁₋₆alkoxycarbonylC₁₋₆alkoxy)C₁₋₆alkoxy, (C₁₋₆alkyl)₂amino, (carboxyC₁₋₆alkoxy)C₁₋₆alkoxy, (carboxyC₃₋₇cycloalkoxy)C₁₋₆alkoxy, (hydroxyC₁₋₆alkyl)₂amino, C₁₋₆alkoxy, carboxyphenylC₁₋₆alkoxy, carboxypiperidinyl, halogen, hydroxy, hydroxyC₁₋₆alkoxy, morpholinyl or pyrrolidinyl;
R⁶ is H, halogen, hydroxyC₁₋₆alkyl or haloC₁₋₆alkyl;
with the proviso that R⁵ and R⁶ are not H simultaneously;
or pharmaceutically acceptable salt, or enantiomer, or diastereomer thereof.

### DETAILED DESCRIPTION OF THE INVENTION

### Definitions

As used herein, the term “C₁₋₆alkyl” denotes to a saturated, linear- or branched chain alkyl group containing 1 to 6, particularly 1 to 4 carbon atoms, for example methyl, ethyl, propyl, isopropyl, 1-butyl, 2-butyl, tert-butyl and the like. Particular “C₁₋₆alkyl” groups are methyl, ethyl, isopropyl and tert-butyl. More particularly, “C₁₋₆alkyl” group is methyl.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclopropyl, cyclobutyl or cyclopentyl.

The term “C₁₋₆alkoxy” denotes to a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy, ethoxy and propoxy. More particularly, “C₁₋₆alkoxy” group is methoxy or ethoxy.

The term “C₃₋₇cycloalkoxy” denotes to a group C₃₋₇cycloalkyl-O—, wherein the “C₃₋₇cycloalkyl” is as defined above; for example cyclopropoxy, cyclobutoxy, cyclopentoxy.

Particular “C₃₋₇cycloalkoxy” group is cyclobutoxy.

The term “halogen” denotes to fluorine, chlorine, bromine or iodine.

The term “carbonyl” denotes to the group —C(O)—.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monofluoro-, monochloro-, difluoro- or trifluoro-methyl, -ethyl or -propyl, for example chloroethyl, 3,3,3-trifluoropropyl, 2-fluoroethyl, 2,2,2-trifluoroethyl, fluoromethyl, difluoromethyl, trifluoromethyl and trifluoroethyl.

The term “enantiomer” denotes two stereoisomers of a compound which are non-superimposable mirror images of one another.

The term “diastereomer” denotes a stereoisomer with two or more centers of chirality and whose molecules are not mirror images of one another. Diastereomers have different physical properties, e.g. melting points, boiling points, spectral properties, and reactivities.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

cccDNA Inhibitors

The present invention provides (i) a compound having the general formula (I):

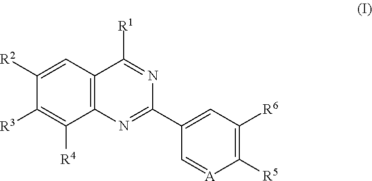

wherein,
A is CH or N;
R¹ is H, (C₁₋₆alkyl)₂aminoC₁₋₆alkyl, C₁₋₆alkoxy, C₁₋₆alkoxyC₁₋₆alkyl, C₁₋₆alkoxycarbonyl, C₁₋₆alkyl, C₁₋₆alkyl-C(O)O—C₁₋₆alkyl-, carbamoyl, carboxy, haloC₁₋₆alkyl, haloC₁₋₆alkylaminocarbonyl, hydroxy, hydroxyC₁₋₆alkyl, hydroxyC₁₋₆alkylaminocarbonyl, morpholinylC₁₋₆alkyl or morpholinylcarbonyl;
R² is H or halogen;
R³ is H, halogen or haloC₁₋₆alkyl;
R⁴ is H or halogen;
R⁵ is H, (C₁₋₆alkoxycarbonyl)phenylC₁₋₆alkoxy, (C₁₋₆alkoxycarbonyl)piperidinyl, (C₁₋₆alkoxycarbonylC₁₋₆alkoxy)C₁₋₆alkoxy, (C₁₋₆alkyl)₂amino, (carboxyC₁₋₆alkoxy)C₁₋₆alkoxy, (carboxyC₃₋₇cycloalkoxy)C₁₋₆alkoxy, (hydroxyC₁₋₆alkyl)₂amino, C₁₋₆alkoxy, carboxyphenylC₁₋₆alkoxy, carboxypiperidinyl, halogen, hydroxy, hydroxyC₁₋₆alkoxy, morpholinyl or pyrrolidinyl;
R⁶ is H, halogen, hydroxyC₁₋₆alkyl or haloC₁₋₆alkyl;
with the proviso that R⁵ and R⁶ are not H simultaneously;
or pharmaceutically acceptable salt, or enantiomer, or diastereomer thereof.

A further embodiment of the present invention is (ii) a compound of formula (I), wherein, A is CH or N;
R¹ is H, acetoxymethyl, carbamoyl, carboxy, chloroethylaminocarbonyl, dimethylaminomethyl, ethoxycarbonyl, hydroxy, hydroxyethylaminocarbonyl, hydroxymethyl, methoxy, methoxycarbonyl, methoxymethyl, methyl, morpholinylcarbonyl, morpholinylmethyl or trifluoromethyl;
R² is H or chloro;
R³ is H, bromo or trifluoromethyl;
R⁴ is H or chloro;
R⁵ is H, (carboxycyclobutoxy)ethoxy, (carboxymethoxy)ethoxy, (hydroxyethyl)₂amino, (methoxycarbonyl)phenylmethoxy, (methoxycarbonyl)piperidinyl, (methoxyoxoethoxy)ethoxy, bromo, carboxyphenylmethoxy, carboxypiperidinyl, chloro, dimethylamino, ethoxy, hydroxy, hydroxyethoxy, methoxy, morpholinyl, pyrrolidinyl;
R⁶ is H, bromo, hydroxymethyl or trifluoromethyl;
with the proviso that R⁵ and R⁶ are not H simultaneously;
or pharmaceutically acceptable salt, or enantiomer, or diastereomer thereof.

A further embodiment of the present invention is (iii) a compound of formula (I) or pharmaceutically acceptable salt, or enantiomer, or diastereomer thereof, wherein R¹ is H, carbamoyl, carboxy, hydroxy, hydroxyC₁₋₆alkylaminocarbonyl, hydroxyC₁₋₆alkyl, C₁₋₆alkyl or morpholinylcarbonyl.

A further embodiment of the present invention is (iv) a compound of formula (I) or pharmaceutically acceptable salt, or enantiomer, or diastereomer thereof, wherein R¹ is H, carbamoyl, carboxy, hydroxy, hydroxyethylaminocarbonyl, hydroxymethyl, methyl or morpholinylcarbonyl.

A further embodiment of the present invention is (v) a compound of formula (I) or pharmaceutically acceptable salt, or enantiomer, or diastereomer thereof, wherein R² is H.

A further embodiment of the present invention is (vi) a compound of formula (I), or pharmaceutically acceptable salt, or enantiomer, or diastereomer thereof, wherein R⁵ is H, (C₁₋₆alkoxycarbonyl)piperidinyl, (C₁₋₆alkoxycarbonylC₁₋₆alkoxy)C₁₋₆alkoxy, (C₁₋₆alkyl)₂amino, (carboxyC₁₋₆alkoxy)C₁₋₆alkoxy, (carboxyC₃₋₇cycloalkoxy)C₁₋₆alkoxy, (hydroxyC₁₋₆alkyl)₂amino, C₁₋₆alkoxy, carboxyphenylC₁₋₆alkoxy, halogen, hydroxyC₁₋₆alkoxy or morpholinyl.

A further embodiment of the present invention is (vii) a compound of formula (I), or pharmaceutically acceptable salt, or enantiomer, or diastereomer thereof, wherein R⁵ is H, (carboxycyclobutoxy)ethoxy, (carboxymethoxy)ethoxy, (hydroxyethyl)₂amino, (methoxycarbonyl)piperidinyl, (methoxyoxoethoxy)ethoxy, bromo, carboxyphenylmethoxy chloro, dimethylamino, hydroxyethoxy, methoxy or morpholinyl.

In another embodiment (viii) of the present invention, particular compounds of the present invention are selected from:
2-(4-Bromophenyl)-8-chloroquinazoline;
2-(3-Bromophenyl)-8-chloroquinazoline;
7-Bromo-4-methyl-2-[3-(trifluoromethyl)phenyl]quinazoline;
2-(3-Bromophenyl)-6-chloro-4-methyl-7-(trifluoromethyl)quinazoline;
4-(8-Chloroquinazolin-2-yl)phenol;
Methyl 3-((4-(8-chloroquinazolin-2-yl)phenoxy)methyl)benzoate;
3-((4-(8-Chloroquinazolin-2-yl)phenoxy)methyl)benzoic acid;
3-((4-(8-Chloro-4-methylquinazolin-2-yl)phenoxy)methyl)benzoic acid;
2-[4-(8-Chloro-4-methyl-quinazolin-2-yl)phenoxy]ethanol;
Methyl 2-[2-[4-(8-chloro-4-methyl-quinazolin-2-yl)phenoxy]ethoxy]acetate;
2-[2-[4-(8-Chloro-4-methyl-quinazolin-2-yl)phenoxy]ethoxy]acetic acid;
cis-3-[2-[4-(8-Chloro-4-methyl-quinazolin-2-yl)phenoxy]ethoxy]cyclobutanecarboxylic acid;
8-Chloro-2-(4-chlorophenyl)-4-methylquinazoline;
2-(2-(4-(4-Methyl-7-(trifluoromethyl)quinazolin-2-yl)phenoxy)ethoxy)acetic acid;
3-(2-(4-(4-Methyl-7-(trifluoromethyl)quinazolin-2-yl)phenoxy)ethoxy) cyclobutanecarboxylic acid;
2-(3-Bromophenyl)-4-(methoxymethyl)-7-(trifluoromethyl)quinazoline;
1-(2-(3-Bromophenyl)-7-(trifluoromethyl)quinazolin-4-yl)-N,N-dimethylmethanamine;
4-((2-(3-Bromophenyl)-7-(trifluoromethyl)quinazolin-4-yl)methyl)morpholine;
2-(2-(3-Bromobenzamido)-4-(trifluoromethyl)phenyl)-2-oxoethyl acetate;
(2-(3-Bromophenyl)-7-(trifluoromethyl)quinazolin-4-yl)methanol;
(8-Chloro-2-(4-chlorophenyl)quinazolin-4-yl)methanol;
(3-(4-Methyl-7-(trifluoromethyl)quinazolin-2-yl)phenyl)methanol;
8-Chloro-2-(6-chloropyridin-3-yl)-4-methylquinazoline;
8-Chloro-2-(6-chloropyridin-3-yl)quinazoline;
5-(8-Chloroquinazolin-2-yl)pyridin-2-ol;
5-(8-Chloroquinazolin-2-yl)-N,N-dimethylpyridin-2-amine;
4-(5-(8-Chloroquinazolin-2-yl)pyridin-2-yl)morpholine;
2,2′-((5-(8-Chloroquinazolin-2-yl)pyridin-2-yl)azanediyl)diethanol;
Methyl 1-(5-(8-chloroquinazolin-2-yl)pyridin-2-yl)piperidine-4-carboxylate;
1-(5-(8-chloroquinazolin-2-yl)pyridin-2-yl)piperidine-4-carboxylic acid;
2-(3-Bromo-phenyl)-7-trifluoromethyl-quinazoline-4-carboxylic acid;
Methyl 2-(3-bromophenyl)-7-(trifluoromethyl)quinazoline-4-carboxylate;
8-Chloro-2-(4-methoxyphenyl)quinazoline-4-carboxylic acid;
8-chloro-2-(4-methoxyphenyl)quinazoline-4-carboxamide;
8-Chloro-N-(2-hydroxyethyl)-2-(4-methoxyphenyl)quinazoline-4-carboxamide;
8-Chloro-2-(4-methoxyphenyl)quinazoline;
2-(4-Bromophenyl)-8-chloroquinazoline-4-carboxylic acid; ethyl 2-(4-bromophenyl)-8-chloro-quinazoline-4-carboxylate;
2-(4-Bromophenyl)-8-chloro-N-(2-chloroethyl)quinazoline-4-carboxamide;
2-(4-Bromophenyl)-8-chloro-4-methyl-quinazoline;
2-[4-(8-Chloroquinazolin-2-yl)phenoxy]ethanol;
cis-3-[2-[4-(8-Chloroquinazolin-2-yl)phenoxy]ethoxy]cyclobutanecarboxylic acid;
2-(4-Bromophenyl)-7-(trifluoromethyl)quinazoline-4-carboxylic acid;
[2-(4-Bromophenyl)-7-(trifluoromethyl)quinazolin-4-yl]-morpholino-methanone;
8-Chloro-2-(6-chloro-3-pyridyl)quinazoline-4-carboxylic acid;
8-chloro-2-(6-chloro-3-pyridyl)quinazoline-4-carboxamide;
8-Chloro-2-(6-methoxy-3-pyridyl)quinazoline-4-carboxylic acid;
8-Chloro-2-(6-ethoxy-3-pyridyl)quinazoline-4-carboxylic acid;
8-Chloro-2-(6-pyrrolidin-1-yl-3-pyridyl)quinazoline-4-carboxylic acid;
cis-2-[6-[2-(3-Carboxycyclobutoxy)ethoxy]-3-pyridyl]-8-chloro-quinazoline-4-carboxylic acid;
2-[2-[[5-(8-Chloroquinazolin-2-yl)-2-pyridyl]oxy]ethoxy]acetic acid;
8-Chloro-2-(6-chloro-3-pyridyl)-N-(2-hydroxyethyl)quinazoline-4-carboxamide;
[8-Chloro-2-(6-chloro-3-pyridyl)quinazolin-4-yl]-morpholino-methanone;
cis-3-[2-[[5-(8-Chloro-4-hydroxy-quinazolin-2-yl)-2-pyridyl]oxy]ethoxy]cyclobutanecarboxylic acid;
3-(2-(4-(8-Chloro-4-methoxyquinazolin-2-yl)phenoxy)ethoxy)cyclobutanecarboxylic acid; and
3-[2-[4-[8-chloro-4-(trifluoromethyl)quinazolin-2-yl]phenoxy]ethoxy]cyclobutanecarboxylic acid;

or pharmaceutically acceptable salt, or enantiomer, or diastereomer thereof.

### Synthesis

The compounds of the present invention can be prepared by any conventional means.

Suitable processes for synthesizing these compounds as well as their starting materials are provided in the schemes below and in the subsequent examples. All substituents, in particular, R¹ to R⁸, X, G₁, G₂, A and Q are defined as below unless otherwise indicated. Furthermore, unless explicitly otherwise stated, all reactions, reaction conditions, abbreviations and symbols have the meanings well known to a person of ordinary skill in the art.

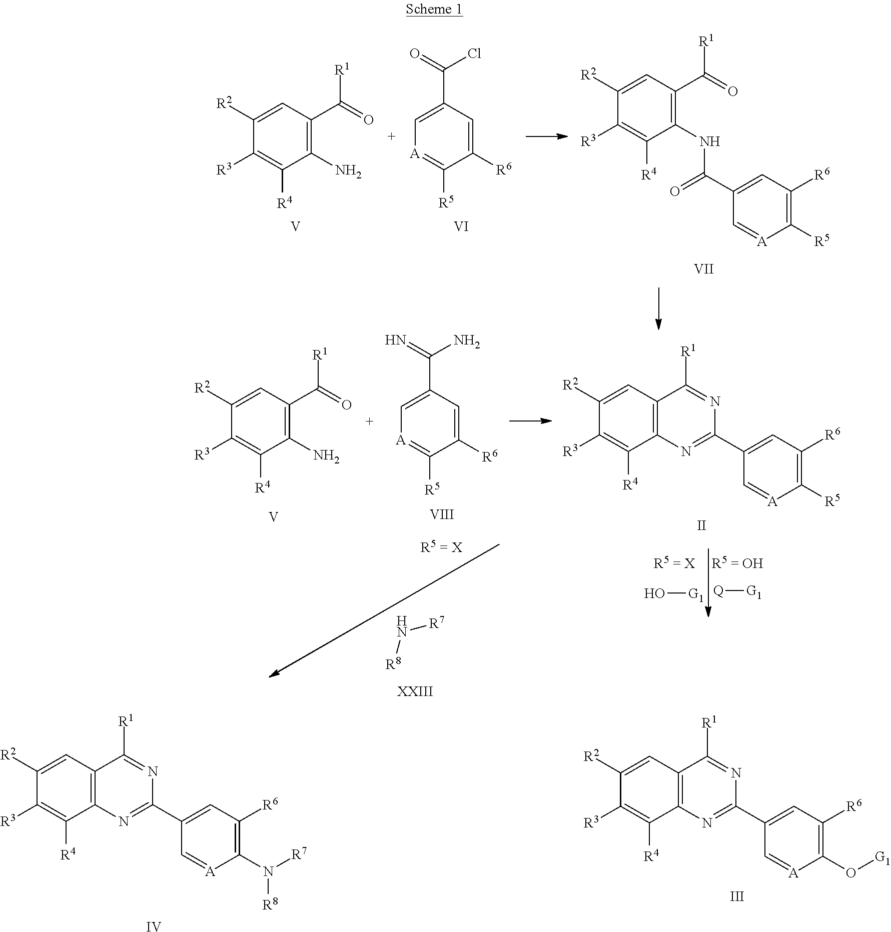

wherein X is halogen; Q is OTf, OMs or halogen; G₁ is (carboxyC₃₋₇cycloalkoxy)C₁₋₆alkyl, (carboxyC₁₋₆alkoxy)C₁₋₆alkyl, (C₁₋₆alkoxycarbonylC₁₋₆alkoxy)C₁₋₆alkyl, carboxyphenylC₁₋₆alkyl, hydroxyC₁₋₆alkyl or C₁₋₆alkyl; R⁷ and R⁸ are independently selected from H, C₁₋₆alkyl or hydroxyC₁₋₆alkyl, or R⁷ and R⁸ together with the nitrogen they are attached to form a 3-8 membered ring.

Compounds of formula II can be prepared by direct cyclization of compounds of formula V with arylamidine VIII under microwave irradiation. Alternatively, the synthesis of compounds of formula II can be prepared from the acylation of compounds of formula V with aryl carbonyl chloride VI, followed by intramolecular cyclization of compounds of formula VII in the presence of an ammonium salt, such as ammonium acetate, under microwave irradiation.

Compounds of formula IV can be prepared by amination of compounds of formula II (when R⁵ is X) with various amines XXIII under microwave irradiation. Compounds of formula III can be prepared by substitution of compounds of formula II (when R⁵ is X) with HO-G₁-O-G₂ in the presence of a base, such as sodium hydride, or alkylation of compounds of formula II (when R⁵ is OH) with Q-G₁-O-G₂ in the presence of a base, such as K₂CO₃.

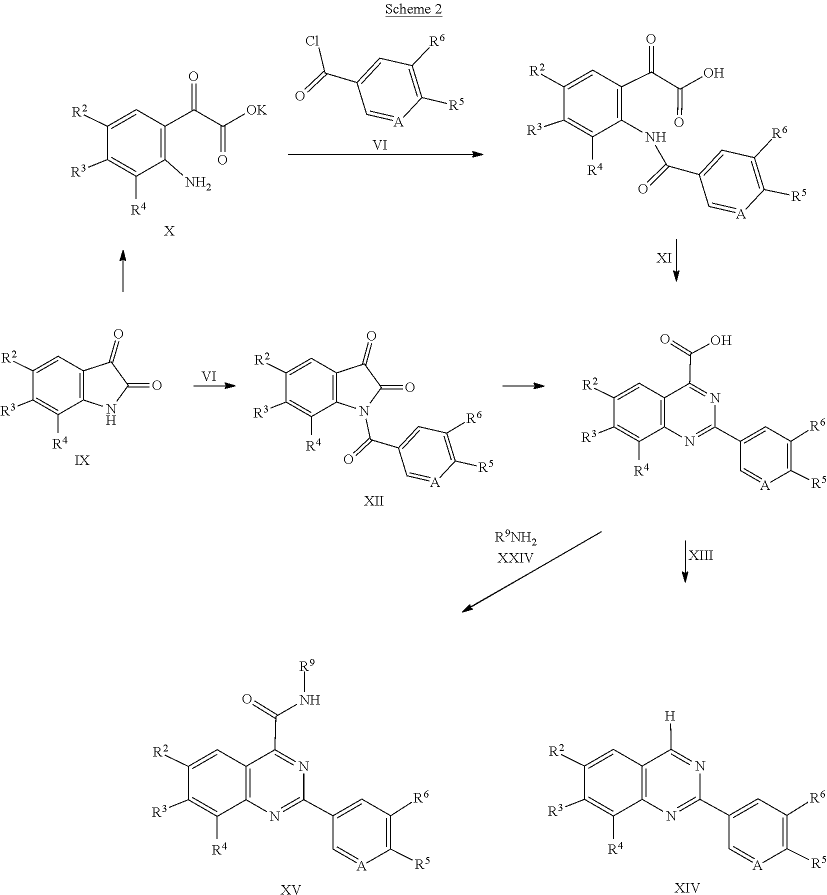

wherein R⁹ is hydroxyC₁₋₆alkyl, haloC₁₋₆alkyl or C₁₋₆alkoxyC₁₋₆alkyl.

Compounds of formula XIII can be prepared starting from hydrolysis of intermediate IX, followed by acylation with benzoyl chloride VI to give intermediate XI, then the intramolecular cyclization of intermediate XI with an ammonium salt, such as acetate ammonium, under microwave irradiation affords compounds of formula XIII. Alternatively, compounds of formula XIII can be prepared from acylation of compounds of formula IX which gives compound of formula XII, followed by ring rearrangement reaction with an ammonium salt, such as ammonium acetate, under microwave irradiation.

Compounds of formula XV can be prepared by coupling reaction of compounds of formula XIII with different amines XXIV in the presence of coupling reagents, such as HATU and oxalyl chloride.

Compounds of formula XIV can be prepared by decarboxylation of compounds of formula XIII. This reaction can occur in the presence of decarboxylation reagent, such as ethylene glycol, under microwave irradiation or in the presence of a catalyst, such as cupric chloride.

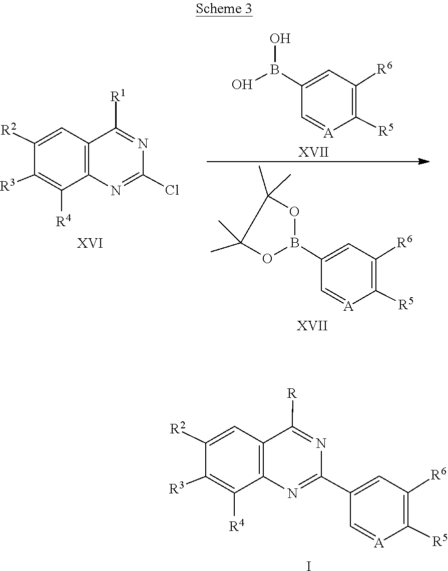

Compounds of formula I also can be prepared by direct Suzuki crossing coupling reaction of intermediate XVI with aryl boric acid XVII or borate ester XVIII in the presence of a catalyst, such as Pd(PPh₃)₄.

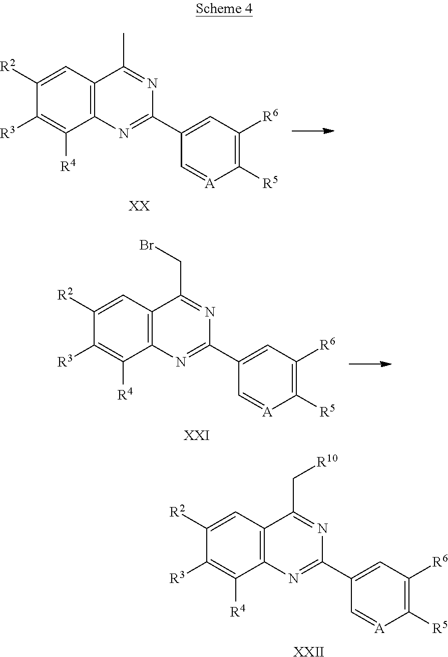

wherein R¹⁰ is (C₁₋₆alkyl)₂amino, morpholinyl or C₁₋₆alkoxy.

Compounds of formula XXII can be prepared by bromination of compounds of formula XX in the present of bromination reagents, such as CuBr₂, followed by substitution reaction using different amines or hydrolysis using a base, such as potassium acetate and potassium hydroxide.

This invention also relates to a process for the preparation of a compound of formula (I) comprising any one of the following steps:
(a) intramolecular cyclization of compounds of formula (VII),

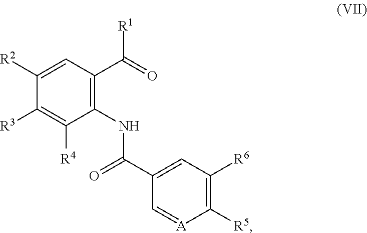

in the presence of an ammonium salt;
(b) cyclization of compounds of formula (V),

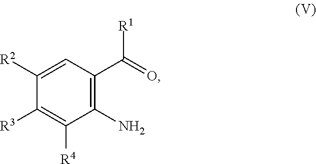

and arylamidine (VIII),

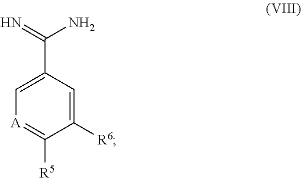

(c) substitution of compounds of formula (II),

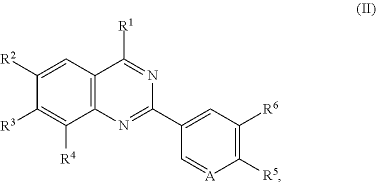

with HO-G₁ in the presence of a base, when R⁵ is X;
(d) substitution of compounds of formula (II),

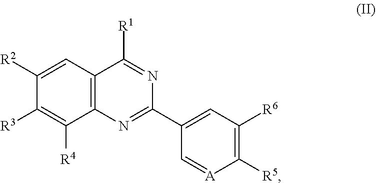

with amine (XXIII), when R⁵ is X;
(e) substitution of compounds of formula (II),

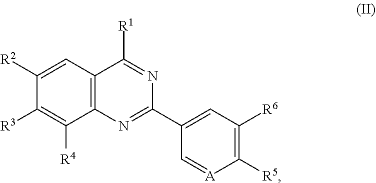

with Q-G₁ in the presence of a base, when R⁵ is OH;
(f) intramolecular cyclization of compounds of formula (XI),

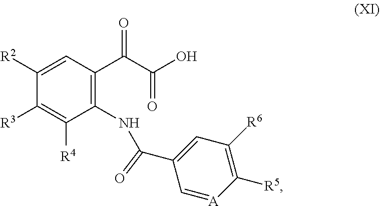

in the presence of an ammonium salt;
(g) ring rearrangement reaction of compounds of formula (XII),

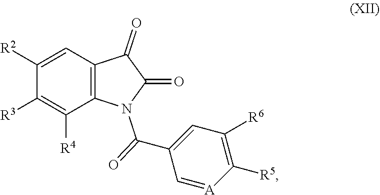

in the presence of an ammonium salt;
(h) coupling reaction of compounds of formula (XIII),

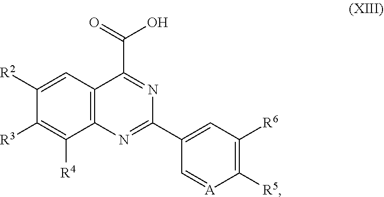

with amines (XXIV) in the presence of coupling reagents;
(i) decarboxylation of a compounds of formula (XIII),

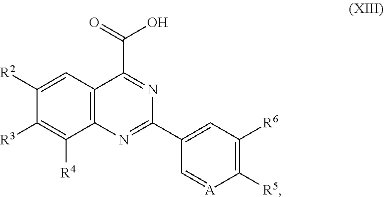

in the presence of decarboxylation reagents;
(j) Suzuki crossing coupling reaction of intermediate (XVI),

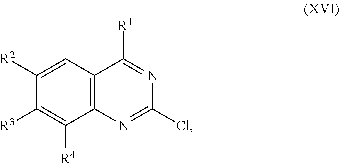

with aryl boric acid (XVII) or borate ester (XVIII) in the presence of a catalyst;
(k) substitution reaction of compounds of formula (XXI),

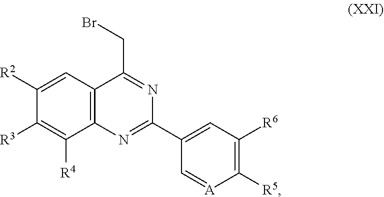

with amines or hydrolysis of compounds of formula (XXI) with a base;
wherein R¹, R², R³, R⁴, R⁵, R⁶, A and G₁ are defined as above;
the ammonium salt in step (a), (f), (g) can be for example ammonium acetate;
the base in step (c), (e), can be for example sodium hydride, K₂CO₃;
the coupling reagent in step (h) can be for example HATU and oxalyl chloride;
the decarboxylation reagents in step (i) can be for example ethylene glycol;
the catalyst in step (j) can be for example cupric chloride;
the base in step (k) can be for example potassium acetate and potassium hydroxide.

A compound of formula (I) when manufactured according to the above process is also an object of the invention.

### Pharmaceutical Compositions and Administration

The invention also relates to a compound of formula (I) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I), or pharmaceutically acceptable salt or enantiomer or diastereomer thereof.

In a further embodiment includes a pharmaceutical composition comprising a compound of formula (I), or pharmaceutically acceptable salt or enantiomer or diastereomer thereof, together with a pharmaceutically acceptable carrier or excipient.

Another embodiment includes a pharmaceutical composition comprising a compound of formula (I), or pharmaceutically acceptable salt or enantiomer or diastereomer thereof for use in the treatment of HBV infection.

### Indications and Methods of Treatment

The compounds of the invention can inhibit HBV cccDNA and have anti-HBV activity. Accordingly, the compounds of the invention are useful for the treatment or prophylaxis of HBV infection.

The invention relates to the use of a compound of formula (I) for the inhibition of HBV cccDNA.

The invention also relates to the use of a compound of formula (I) for the inhibition of HBeAg.

The invention further relates to the use of a compound of formula (I) for the inhibition of HBsAg.

The invention relates to the use of a compound of formula (I) for the inhibition of HBV DNA.

The invention relates to the use of a compound of formula (I) for the treatment or prophylaxis of HBV infection.

The use of a compound of formula (I) for the preparation of medicaments useful in the treatment or prophylaxis diseases that are related to HBV infection is an object of the invention.

The invention relates in particular to the use of a compound of formula (I) for the preparation of a medicament for the treatment or prophylaxis of HBV infection.

Another embodiment includes a method for the treatment or prophylaxis of HBV infection, which method comprises administering an effective amount of a compound of Formula (I), or enantiomers, diastereomers, prodrugs or pharmaceutically acceptable salts thereof.

### EXAMPLES

The invention will be more fully understood by reference to the following examples. They should not, however, be construed as limiting the scope of the invention.

Abbreviations used herein are as follows:
AcOH: acetic acid
BBr₃: boron tribromide
DCM: dichloromethane
DMAP: 4-dimethylaminopyridine
DMA: Acetic acid dimethylamide
DME: dimethoxyethane
DMF: N,N-dimethylformamide
DPPF: 1,1′-Bis(diphenylphosphino)ferrocene
EC₅₀: the molar concentration of an inhibitor, which produces 50% of the maximum possible response for that inhibitor.
FBS: fetal bovine serum
HATU: 1-[Bis(dimethylamino)methylene]-1H-1,2,3-triazolo[4,5-b]pyridinium 3-oxid hexafluorophosphate
H₂O₂: hydrogen peroxide
HPLC: high performance liquid chromatography
hr(s): hour(s)
min: minute
MS (ESI): mass spectroscopy (electron spray ionization)
Ms: methylsulfonyl
NCS: N-chlorosuccinimide
NMP: N-methyl-2-pyrrolidone
obsd.: observed
PE: petroleum ether
PPh₃: triphenylphosphine
rt: room temperature
Tf: trifluoromethanesulfonyl
TFA: trifluoroacetic acid
THF: Tetrahydrofuran
TMS: trimethylsilyl
Ts: p-tolylsulfonyl
δ: chemical shift

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):
Acidic condition: A: 0.1% formic acid and 1% acetonitrile in H₂O; B: 0.1% formic acid in acetonitrile;
Basic condition: A: 0.05% NH₃—H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under argon atmosphere. Reagents were used as received from commercial suppliers without further purification unless otherwise noted.

### PREPARATIVE EXAMPLES

### Example 1

### 2-(4-Bromophenyl)-8-chloroquinazoline

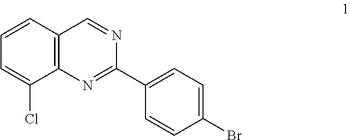 [SMILES: Clc1cccc2cnc(-c3ccc(Br)cc3)nc12]

### Preparation of 2-(4-bromophenyl)-8-chloroquinazoline

A mixture of 2-amino-3-chlorobenzaldehyde (200 mg, 1.29 mmol) and 4-bromobenzimidamide hydrochloride (303 mg, 1.29 mmol) in NMP (3 mL) was heated under microwave irradiation at 210° C. for 1 hr. After being cooled to rt, the reaction mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:10 to 100:50 (v/v) to give 2-(4-bromophenyl)-8-chloroquinazoline (300 mg, Example 1) as a solid. ¹H NMR (400 MHz, DMSO-d₆) δ ppm 9.78-9.81 (m, 1H), 8.51-8.57 (m, 2H), 8.24 (dd, J=1.22, 7.58 Hz, 1H), 8.19 (dd, J=1.22, 8.07 Hz, 1H), 7.79-7.85 (m, 2H), 7.75 (t, J=7.82 Hz, 1H). MS obsd. (ESI⁺) [(M+H)⁺]: 319.0.

### Example 2

### 2-(3-Bromophenyl)-8-chloroquinazoline

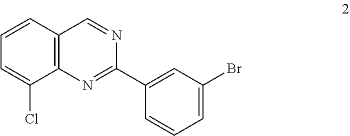 [SMILES: Clc1cccc2cnc(-c3cccc(Br)c3)nc12]

### Preparation of 2-(3-bromophenyl)-8-chloroquinazoline

Example 2 was prepared in analogy to Example 1 by using 3-bromobenzimidamide hydrochloride (75.7 mg, 0.321 mmol) instead of 4-bromobenzimidamide hydrochloride. 2-(3-Bromophenyl)-8-chloroquinazoline (Example 2, 19 mg) was obtained as a solid. ¹H NMR (400 MHz, DMSO-d₆) δ ppm 9.82 (s, 1H), 8.73 (t, J=1.83 Hz, 1H), 8.61 (d, J=8.31 Hz, 1H), 8.26 (dd, J=1.22, 7.58 Hz, 1H), 8.21 (dd, J=1.22, 8.07 Hz, 1H), 7.81 (ddd, J=0.98, 2.08, 7.95 Hz, 1H), 7.76 (t, J=7.83 Hz, 1H), 7.59 (t, J=7.95 Hz, 1H). MS obsd. (ESI⁺) [(M+H)⁺]: 319.0.

### Example 3

### 7-Bromo-4-methyl-2-[3-(trifluoromethyl)phenyl]quinazoline

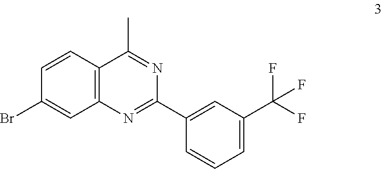 [SMILES: Cc1nc(-c2cccc(C(F)(F)F)c2)nc2cc(Br)ccc12]

### Step 1: Preparation of N-(2-acetyl-5-bromophenyl)-3-(trifluoromethyl)benzamide

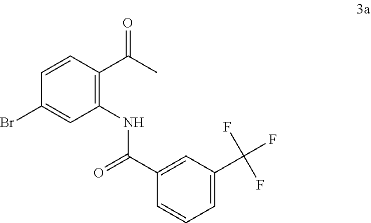 [SMILES: CC(=O)c1ccc(Br)cc1NC(=O)c1cccc(C(F)(F)F)c1]

To a solution of 1-(2-amino-4-bromophenyl)ethanone (300 mg, 1.4 mmol) and TEA (566 mg, 5.61 mmol) in DCM (10 mL) was added 4-(trifluoromethyl)benzoyl chloride (292 mg, 1.4 mmol) and the mixture was then stirred at 25° C. for 4 hrs. After the reaction was completed, the mixture was diluted with DCM (40 mL). The resulting organic solution was washed with 1N aqueous HCl (25 mL), water (25 mL), saturated NaHCO₃ solution (20 mL), brine, and dried over MgSO₄ and then concentrated in vacuo. The residue was suspended in EtOH (10 mL). The precipitate of product was collected and dried to give N-(2-acetyl-5-bromophenyl)-3-(trifluoromethyl)benzamide (250 mg, compound 3a) as a yellow solid. MS obsd. (ESI⁺) [(M+H)⁺]: 386.0.

### Step 2: Preparation of 7-bromo-4-methyl-2-[3-(trifluoromethyl)phenyl]quinazoline

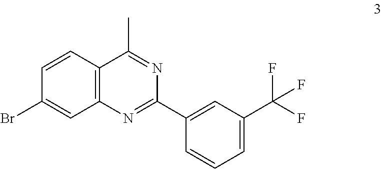 [SMILES: Cc1nc(-c2cccc(C(F)(F)F)c2)nc2cc(Br)ccc12]

A suspension of N-(2-acetyl-5-bromophenyl)-3-(trifluoromethyl)benzamide (compound 3a, 250 mg, 0.647 mmol) and ammonium acetate (2 g, 33.3 mmol) in EtOH (10 mL) was stirred at 80° C. under microwave irradiation for 0.5 hr. The mixture was then concentrated in vacuo. The residue was purified by column chromatography on silica gel using gradient elution with PE-EtOAc from 100:10 to 100:50 (v/v) to give 7-bromo-4-methyl-2-[3-(trifluoromethyl)phenyl]quinazoline (Example 3, 130 mg) as a white solid. ¹H NMR (DMSO-d₆, 400 MHz): δ ppm 8.81-8.87 (m, 2H), 8.35-8.37 (m, 1H), 8.28-8.32 (m, 1H), 7.94-7.99 (m, 1H), 7.90-7.94 (m, 1H), 7.79-7.88 (m, 1H), 3.03 (s, 3H). MS obsd. (ESI⁺) [(M+H)⁺]: 367.3, 369.3.

### Example 4

### 2-(3-Bromophenyl)-6-chloro-4-methyl-7-(trifluoromethyl)quinazoline

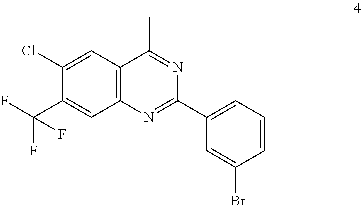 [SMILES: Cc1nc(-c2cccc(Br)c2)nc2cc(C(F)(F)F)c(Cl)cc12]

### Step 1: Preparation of methyl 2-amino-5-chloro-4-(trifluoromethyl)benzoate

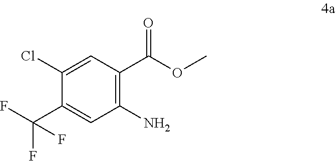 [SMILES: COC(=O)c1cc(Cl)c(C(F)(F)F)cc1N]

A mixture of methyl 2-amino-5-iodo-4-(trifluoromethyl)benzoate (1.2 g, 3.48 mmol) and copper(I) chloride (1.2 g, 12.1 mmol) in NMP (3 mL) was stirred at 150° C. under microwave condition for 0.5 hr. After the reaction was completed, the mixture was quenched by addition of 0.5 N aqueous HCl (20 mL) and extracted with EtOAc (20 mL) three times. The combined organic layer was washed with brine, dried over Na₂SO₄, and concentrated in vacuo. The residue was purified by column chromatography on silica gel using gradient elution with PE-EtOAc from 100:10 to 100:50 (v/v) to give methyl 2-amino-5-chloro-4-(trifluoromethyl)benzoate (compound 4a, 620 mg) as a solid. MS obsd. (ESI⁺) [(M+H)⁺]: 254.0.

### Step 2: Preparation of 2-amino-5-chloro-4-(trifluoromethyl)benzoic acid

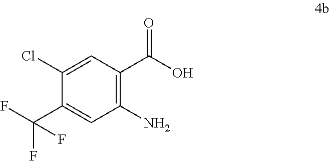 [SMILES: Nc1cc(C(F)(F)F)c(Cl)cc1C(=O)O]

To a solution of methyl 2-amino-5-chloro-4-(trifluoromethyl)benzoate (compound 4a, 620 mg, 2.44 mmol) in the mixed solvent of THF (20 mL), MeOH (5 mL) and water (0.5 mL) was added LiOH (230 mg, 10 mmol), the mixture was then stirred at rt overnight. After the reaction was completed, the mixture was adjusted to pH 6.0 by addition of AcOH (1.2 g, 20 mmol). The mixture was then partitioned between EtOAc (20 mL) and saturated with NH₄Cl (20 mL). The organic layer was separated and the aqueous phase was extracted with EtOAc (20 mL) twice. The combined organic phase was dried over Na₂SO₄ and concentrated in vacuo. The residue was purified by column chromatography on silica gel using gradient elution with PE-EtOAc from 100:10 to 100:50 (v/v) to give 2-amino-5-chloro-4-(trifluoromethyl)benzoic acid (compound 4b, 460 mg) as a yellow solid. MS obsd. (ESI⁺) [(M+H)⁺]: 240.1.

### Step 3: Preparation of 1-[2-amino-5-chloro-4-(trifluoromethyl)phenyl]ethanone

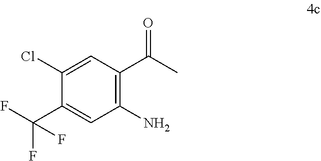

To a suspension of 2-amino-5-chloro-4-(trifluoromethyl)benzoic acid (compound 4b, 340 mg, 1.42 mmol) in THF (5 mL) was added 3 N methyl lithium in DME (11.4 mmol) at −78° C. After addition was completed, the reaction mixture was warmed to rt and stirred for 4 hrs, then quenched by saturated NH₄Cl solution (20 mL) and extracted with EtOAc (50 mL) twice. The combined organic phase was dried over Na₂SO₄ and concentrated in vacuo. The residue was purified by column chromatography on silica gel using gradient elution with PE-EtOAc from 100:10 to 100:50 (v/v) to give 1-(2-amino-5-chloro-4-(trifluoromethyl)phenyl)ethanone (compound 4c, 200 mg) as a yellow solid. MS obsd. (ESI⁺) [(M+H)⁺]: 238.1.

### Step 4: Preparation of N-[2-acetyl-4-chloro-5-(trifluoromethyl)phenyl]-3-bromo-benzamide

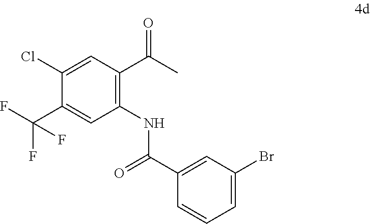 [SMILES: CC(=O)c1cc(Cl)c(C(F)(F)F)cc1NC(=O)c1cccc(Br)c1]

To a solution of 1-(2-amino-5-chloro-4-(trifluoromethyl)phenyl)ethanone (compound 4c, 200 mg, 0.842 mmol) and DMAP (157 mg, 1.3 mmol) in DCM (25 mL) was added 3-bromobenzoyl chloride (175 mg, 0.797 mmol). The mixture was stirred at rt overnight, then washed by 2N aqueous HCl (25 mL), water (50 mL), NaHCO₃ solution (25 mL), and brine in sequence. The organic layer was then dried over MgSO₄ and concentrated in vacuo to give the crude of N-[2-acetyl-4-chloro-5-(trifluoromethyl)phenyl]-3-bromo-benzamide (compound 4d, 350 mg) as a yellow oil, which was used in next step directly. MS obsd. (ESI⁺) [(M+H)⁺]: 420.1.

### Step 5: Preparation of 2-(3-bromophenyl)-6-chloro-4-methyl-7-(trifluoromethyl)quinazoline

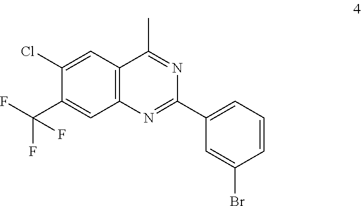 [SMILES: Cc1nc(-c2cccc(Br)c2)nc2cc(C(F)(F)F)c(Cl)cc12]

A mixture of N-[2-acetyl-4-chloro-5-(trifluoromethyl)phenyl]-3-bromo-benzamide (compound 4d, 300 mg, 0.713 mmol) and ammonium acetate (4.5 g, 58.4 mmol) in EtOH (15 mL) was heated under microwave irradiation at 85° C. for 1 hr. After being cooled to rt, the reaction mixture was concentrated in vacuo. The mixture was partitioned between EtOAc (100 mL) and water (100 mL), and the separated aqueous phase was extracted with EtOAc (100 mL) twice. The combined organic phase was dried over Na₂SO₄ and concentrated in vacuo. The residue was suspended in MeOH (15 mL). The suspension was filtered and dried to give 2-(3-bromophenyl)-6-chloro-4-methyl-7-(trifluoromethyl)quinazoline (Example 4, 31.5 mg,) as a light yellow solid. ¹H NMR (DMSO-d₆, 400 MHz): δ ppm 8.70 (s, 2H), 8.54 (s, 2H), 7.76-7.84 (m, 1H), 7.57 (s, 1H), 3.06 (s, 3H). MS obsd. (ESI⁺) [(M+H)⁺]: 401.0.

### Example 5

### 4-(8-Chloroquinazolin-2-yl)phenol

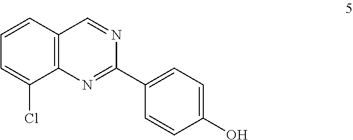 [SMILES: Oc1ccc(-c2ncc3cccc(Cl)c3n2)cc1]

### Preparation of 4-(8-chloroquinazolin-2-yl)phenol

4-(8-Chloroquinazolin-2-yl)phenol (Example 5) was prepared in analogy to Example 1 by using 4-hydroxybenzimidamide hydrochloride (200 mg, 1.16 mmol) instead of 4-bromobenzimidamide hydrochloride. 4-(8-Chloroquinazolin-2-yl)phenol (Example 5, 150 mg) was obtained as a solid. ¹H NMR (400 MHz, DMSO-d₆) δ ppm 10.07-10.14 (m, 1H), 9.68 (s, 1H), 8.41-8.52 (m, 2H), 8.15 (s, 1H), 8.10 (s, 1H), 7.65 (t, J=7.82 Hz, 1H), 6.91-7.00 (m, 2H). MS obsd. (ESI⁺) [(M+H)⁺]: 257.0.

### Example 6

### Methyl 3-((4-(8-chloroquinazolin-2-yl)phenoxy)methyl)benzoate

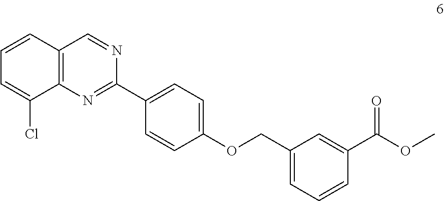 [SMILES: COC(=O)c1cccc(COc2ccc(-c3ncc4cccc(Cl)c4n3)cc2)c1]

### Preparation of methyl 3-((4-(8-chloroquinazolin-2-yl)phenoxy)methyl)benzoate

To a solution of 4-(8-chloroquinazolin-2-yl)phenol (Example 5, 110 mg, 0.429 mmol) in acetonitrile (4 mL) was added K₂CO₃ (88.8 mg, 0.643 μmol) and methyl 3-(bromomethyl)benzoate (118 mg, 0.514 mmol) at rt. The mixture was stirred at 60° C. for 2 hrs. After the reaction was completed, the solvent was removed in vacuo. The residue was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:10 to 100:50 (v/v) to give methyl 3-((4-(8-chloroquinazolin-2-yl)phenoxy)methyl)benzoate (Example 6, 120 mg) as a solid. ¹H NMR (400 MHz, DMSO-d₆) δ ppm 9.72 (s, 1H), 8.58 (d, J=9.05 Hz, 2H), 8.19 (dd, J=1.22, 7.34 Hz, 1H), 8.14 (dd, J=1.22, 8.31 Hz, 1H), 8.10 (s, 1H), 7.95 (d, J=7.83 Hz, 1H), 7.79 (d, J=8.07 Hz, 1H), 7.68 (t, J=7.83 Hz, 1H), 7.55-7.62 (m, 1H), 7.22-7.30 (m, 2H), 5.33 (s, 2H), 3.88 (s, 3H). MS obsd. (ESI⁺) [(M+H)⁺]: 405.1.

### Example 7

### 3-((4-(8-Chloroquinazolin-2-yl)phenoxy)methyl)benzoic acid

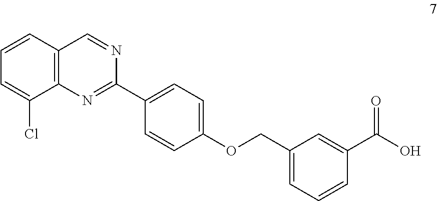 [SMILES: O=C(O)c1cccc(COc2ccc(-c3ncc4cccc(Cl)c4n3)cc2)c1]

### Preparation of 3-((4-(8-chloroquinazolin-2-yl)phenoxy)methyl)benzoic acid

To a solution of methyl 3-((4-(8-chloroquinazolin-2-yl)phenoxy)methyl)benzoate (Example 6, 110 mg, 0.272 mmol) in THF (5 mL) was added 2N LiOH (1 mL) solution at rt, and the mixture was stirred at 50° C. for 1 hr. After the reaction was completed, the mixture was adjusted to pH 4 by addition of 1N Aqueous HCl then partitioned between EtOAc (10 mL) and water (10 mL), and the separated aqueous phase was extracted with EtOAc (10 mL) twice. The combined organic phase was dried over Na₂SO₄ and concentrated in vacuo to give 3-((4-(8-chloroquinazolin-2-yl)phenoxy)methyl)benzoic acid (Example 7, 50 mg) as a solid. ¹H NMR (400 MHz, DMSO-d₆) δ ppm 9.72 (s, 1H), 8.58 (d, J=9.05 Hz, 2H), 8.19 (dd, J=1.22, 7.58 Hz, 1H), 8.14 (dd, J=1.22, 8.07 Hz, 1H), 8.08 (s, 1H), 7.93 (d, J=7.83 Hz, 1H), 7.75 (d, J=7.58 Hz, 1H), 7.68 (t, J=7.83 Hz, 1H), 7.56 (t, J=7.83 Hz, 1H), 7.25 (d, J=9.05 Hz, 2H), 5.32 (s, 2H). MS obsd. (ESI⁺) [(M+H)⁺]: 391.1.

### Example 8

### 3-((4-(8-Chloro-4-methylquinazolin-2-yl)phenoxy)methyl)benzoic acid

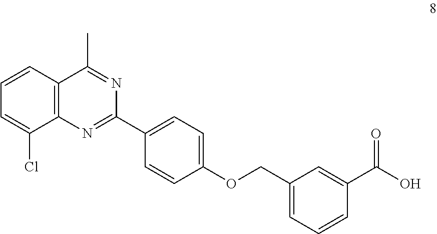 [SMILES: Cc1nc(-c2ccc(OCc3cccc(C(=O)O)c3)cc2)nc2c(Cl)cccc12]

### Step 1: Preparation of 4-(8-chloro-4-methylquinazolin-2-yl)phenol

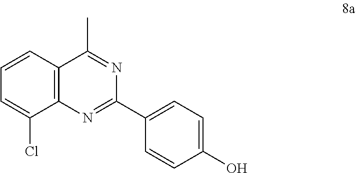 [SMILES: Cc1nc(-c2ccc(O)cc2)nc2c(Cl)cccc12]

A mixture of 1-(2-amino-3-chlorophenyl)ethanone (100 mg, 0.59 mmol) and 4-hydroxybenzimidamide hydrochloride (509 mg, 2.95 mmol) in NMP (3 mL) was heated under microwave irradiation at 210° C. for 1 hr. After being cooled to rt, the reaction mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:20 to 100:100 (v/v) to give 4-(8-chloro-4-methylquinazolin-2-yl)phenol (compound 8a, 200 mg) as a solid. MS obsd. (ESI⁺) [(M+H)⁺]: 271.1.

### Step 2: Preparation of methyl 3-((4-(8-chloro-4-methylquinazolin-2-yl)phenoxy)methyl)benzoate

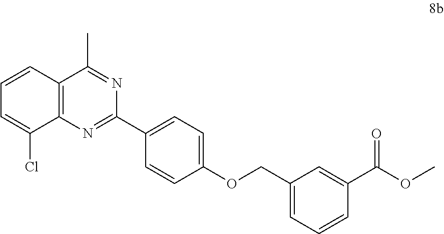 [SMILES: COC(=O)c1cccc(COc2ccc(-c3nc(C)c4cccc(Cl)c4n3)cc2)c1]

Compound 8b was prepared in analogy to Example 6 by using 4-(8-chloro-4-methylquinazolin-2-yl)phenol (compound 8a, 200 mg) instead of 4-(8-chloroquinazolin-2-yl)phenol (Example 5). Methyl 3-((4-(8-chloro-4-methylquinazolin-2-yl)phenoxy)methyl)benzoate (compound 8b, 200 mg) was obtained as a solid. MS obsd. (ESI⁺) [(M+H)⁺]: 419.1.

### Step 3: Preparation of 3-((4-(8-chloro-4-methylquinazolin-2-yl)phenoxy)methyl)benzoic acid

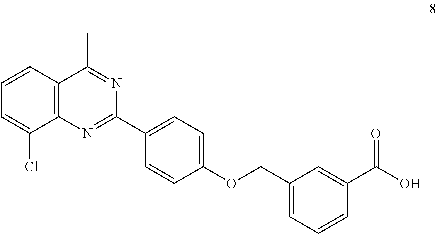 [SMILES: Cc1nc(-c2ccc(OCc3cccc(C(=O)O)c3)cc2)nc2c(Cl)cccc12]

Example 8 was prepared in analogy to Example 7 by using methyl 3-((4-(8-chloro-4-methylquinazolin-2-yl)phenoxy)methyl)benzoate (compound 8b, 200 mg, 0.477 mmol) instead of methyl 3-((4-(8-chloroquinazolin-2-yl)phenoxy)methyl)benzoate (Example 6). 3-((4-(8-Chloro-4-methylquinazolin-2-yl)phenoxy)methyl)benzoic acid (Example 8, 21 mg) was obtained as a solid. ¹H NMR (400 MHz, DMSO-d₆) δ ppm 8.50 (d, J=8.80 Hz, 2H), 8.18 (dd, J=1.10, 8.19 Hz, 1H), 8.07 (dd, J=1.22, 7.58 Hz, 1H), 8.01 (s, 1H), 7.86 (d, J=7.58 Hz, 1H), 7.68 (d, J=7.58 Hz, 1H), 7.56 (t, J=7.95 Hz, 1H), 7.46-7.52 (m, 1H), 7.17 (d, J=9.05 Hz, 2H), 5.24 (s, 2H), 2.94 (s, 3H). MS obsd. (ESI⁺) [(M+H)⁺]: 405.1.

### Example 9

### 2-[4-(8-Chloro-4-methyl-quinazolin-2-yl)phenoxy]ethanol

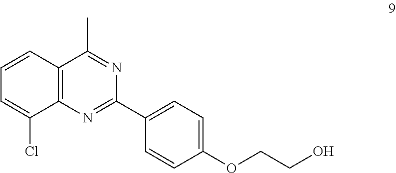 [SMILES: Cc1nc(-c2ccc(OCCO)cc2)nc2c(Cl)cccc12]

### Step 1: Preparation of 2-[4-(4,4,5,5-tetramethyl-1,3,2-dioxaborolan-2-yl)phenoxy]ethanol

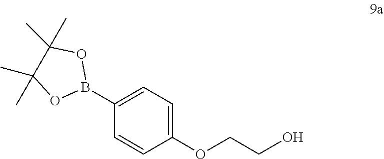 [SMILES: CC1(C)OB(c2ccc(OCCO)cc2)OC1(C)C]

A mixture of 2-(4-bromophenoxy)ethanol (4.34 g, 20 mmol), 4,4,4′,4′,5,5,5′,5′-octamethyl-2,2′-bi(1,3,2-dioxaborolane) (5.59 g, 22 mmol), PdCl₂(DPPF)-CH₂Cl₂ adduct (439 mg, 0.600 mmol), and potassium acetate (5.89 g, 60 mmol) in DMSO (50 mL) was heated to 90° C. for 4 hrs. After being cooled to rt, the mixture was diluted with EtOAc (300 mL), and washed with water (100 mL) twice. The organic layer was separated and concentrated in vacuo. The residue was purified by column chromatography on silica gel using gradient elution with PE-EtOAc from 100:10 to 100:50 (v/v) to give 2-[4-(4,4,5,5-tetramethyl-1,3,2-dioxaborolan-2-yl)phenoxy]ethanol (compound 9a, 4.3 g) as a light yellow oil. MS obsd. (ESI⁺) [(M+H)⁺]: 265.2.

### Step 2: Preparation of N-(2-acetyl-6-chloro-phenyl)-2,2,2-trichloro-acetamide

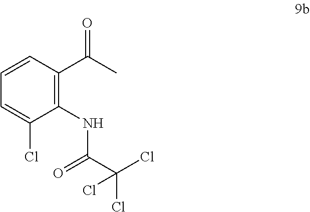 [SMILES: CC(=O)c1cccc(Cl)c1NC(=O)C(Cl)(Cl)Cl]

To a solution of 1-(2-amino-3-chlorophenyl)ethanone (5.5 g, 32.4 mmol) and DMAP (0.7 g, 5.73 mmol) in DCM (50 mL) was added 2,2,2-trichloroacetyl chloride (7.08 g, 38.9 mmol) dropwise at 0° C. The mixture was stirred for 2 hrs before it was diluted with EtOAc (100 mL). The mixture was washed with water (30 mL) and 0.5 N aqueous HCl (30 mL) separately. The organic layer was separated, dried over anhydrous Na₂SO₄ and concentrated in vacuo. The residue was purified by column chromatography on silica gel using gradient elution with PE-EtOAc from 100:10 to 100:50 (v/v) to give N-(2-acetyl-6-chloro-phenyl)-2,2,2-trichloro-acetamide (compound 9b, 9.36 g, 92.1%) as a light yellow solid. MS obsd. (ESI⁺) [(M+H)⁺]: 313.9.

### Step 3: Preparation 8-chloro-4-methyl-1H-quinazolin-2-one

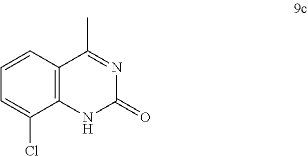

To a solution of N-(2-acetyl-6-chloro-phenyl)-2,2,2-trichloro-acetamide (compound 9b, 9.36 g, 29.8 mmol) in THF (20 mL) was added to a solution of ammonium hydroxide (15 mL, 385 mmol). The mixture was stirred overnight. After evaporated off the solvent, 8-chloro-4-methyl-1H-quinazolin-2-one (compound 9c, 5.78 g) was obtained as a white solid which was used in next step without further purification. MS obsd. (ESI⁺) [(M+H)⁺]: 195.1.

### Step 4: Preparation of 2,8-dichloro-4-methyl-quinazoline

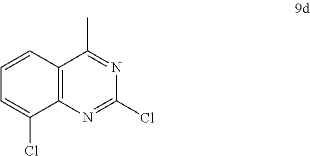

To a suspension solution of 8-chloro-4-methyl-1H-quinazolin-2-one (compound 9c, 3.5 g, 18 mmol,) in POCl₃ (19.7 g, 12 mL, 129 mmol) was added N,N-diethylaniline (2.68 g, 18 mmol), and the mixture was heated to 80° C. for 3 hrs. After being cooled to rt, the mixture was diluted with DCM (20 mL) and quenched with ice-water carefully. The organic layer was separated, and the aqueous phase was extracted with DCM (20 mL). The combined organic phase was concentrated in vacuo. The residue was purified by column chromatography on silica gel using gradient elution with PE-EtOAc from 100:10 to 100:50 (v/v) to give 2,8-dichloro-4-methyl-quinazoline (compound 9d, 2.4 g) as a light yellow solid. MS obsd. (ESI⁺) [(M+H)⁺]: 213.2.

### Step 5: Preparation of 2-[4-(8-chloro-4-methyl-quinazolin-2-yl)phenoxy]ethanol

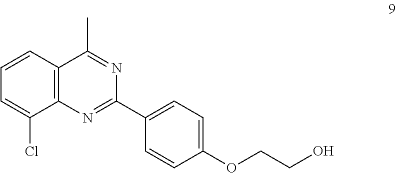 [SMILES: Cc1nc(-c2ccc(OCCO)cc2)nc2c(Cl)cccc12]

A mixture of 2,8-dichloro-4-methyl-quinazoline (compound 9d, 800 mg, 3.75 mmol), 2-[4-(4,4,5,5-tetramethyl-1,3,2-dioxaborolan-2-yl)phenoxy]ethanol (compound 9a, 992 mg, 3.75 mmol), Pd(Ph₃P)₄ (130 mg, 0.113 mmol) and Cs₂CO₃ (1.84 g, 5.63 mmol) in toluene (7 mL), MeOH (1 mL) and water (1 mL) was charged with N₂ and heated under microwave irradiation at 100° C. for 3 hrs. After being cooled to rt, the mixture was extracted with EtOAc (50 mL) and washed with water (10 mL). The organic layer was separated and concentrated in vacuo. The residue was purified by column chromatography on silica gel using gradient elution with PE-EtOAc from 100:10 to 100:50 (v/v) to give 2-[4-(8-chloro-4-methyl-quinazolin-2-yl)phenoxy]ethanol (Example 9, 980 mg, 83.2%) as a light yellow solid. ¹H NMR (DMSO-d₆, 400 MHz)): δ ppm 8.51-8.58 (m, 2H), 8.25 (dd, J=1.22, 8.31 Hz, 1H), 8.14 (dd, J=1.22, 7.58 Hz, 1H), 7.58-7.67 (m, 1H), 7.10-7.17 (m, 2H), 4.92 (t, J=5.50 Hz, 1H), 4.10 (t, J=5.01 Hz, 2H), 3.77 (q, J=5.38 Hz, 2H), 3.00 (s, 3H). MS obsd. (ESI⁺) [(M+H)⁺]: 315.1.

### Example 10

### Methyl 2-[2-[4-(8-chloro-4-methyl-quinazolin-2-yl)phenoxy]ethoxy]acetate

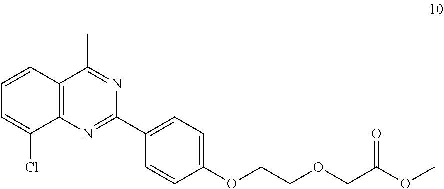 [SMILES: COC(=O)COCCOc1ccc(-c2nc(C)c3cccc(Cl)c3n2)cc1]

### Preparation of methyl 2-[2-[4-(8-chloro-4-methyl-quinazolin-2-yl)phenoxy]ethoxy]acetate

To a solution of 2-[4-(8-chloro-4-methyl-quinazolin-2-yl)phenoxy]ethanol (Example 9, 700 mg, 2.22 mmol) and 2,6-lutidine (357 mg, 3.34 mmol) in DCM (5 mL) was added trifluoromethanesulfonic anhydride (941 mg, 3.34 mmol) dropwise at 0° C. over 30 min. The mixture was diluted with EtOAc (20 mL) and washed with water (10 mL), 0.5 N aqueous HCl (10 mL) and water (10 mL). The organic layer was separated and dried over anhydrous Na₂SO₄, and then concentrated in vacuo. The residue was added to a pre-prepared mixture of methyl 2-hydroxyacetate (401 mg, 4.45 mmol) and sodium hydride (80.1 mg, 3.34 mmol) in THF (5 mL) at 0° C. The mixture was stirred for 1 hr before it was quenched by water, which was extracted with EtOAc (30 mL) and washed with water (10 mL). The organic layer was separated and concentrated in vacuo. The residue was purified by column chromatography on silica gel using gradient elution with PE-EtOAc from 100:10 to 100:50 (v/v) to give methyl 2-[2-[4-(8-chloro-4-methyl-quinazolin-2-yl)phenoxy]ethoxy]acetate (Example 10, 460 mg) as a light yellow solid. ¹H NMR (CHLOROFORM-d, 400 MHz): δ ppm 8.58 (d, J=9.05 Hz, 2H), 7.90 (dd, J=8.31, 1.22 Hz, 1H), 7.85 (dd, J=7.58, 1.22 Hz, 1H), 7.33-7.39 (m, 1H), 6.98 (d, J=8.93 Hz, 2H), 4.16-4.24 (m, 4H), 3.92 (dd, J=5.44, 3.85 Hz, 2H), 3.69 (s, 3H), 2.93 (s, 3H). MS obsd. (ESI⁺) [(M+H)⁺]: 387.1.

### Example 11

### 2-[2-[4-(8-Chloro-4-methyl-quinazolin-2-yl)phenoxy]ethoxy]acetic acid

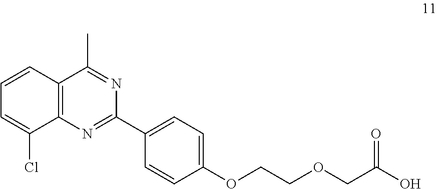 [SMILES: Cc1nc(-c2ccc(OCCOCC(=O)O)cc2)nc2c(Cl)cccc12]

### Preparation of 2-[2-[4-(8-chloro-4-methyl-quinazolin-2-yl)phenoxy]ethoxy]acetic acid

To a solution of methyl 2-[2-[4-(8-chloro-4-methyl-quinazolin-2-yl)phenoxy]ethoxy]acetate (Example 10, 300 mg, 776 μmol) in THF (5 mL) and water (5 mL) was added Lithium hydroxide monohydrate (81.4 mg, 1.94 mmol), and the mixture was stirred for 2 hrs. After THF was removed in vacuo, the remained aqueous solution was acidified by 1 N aqueous HCl to pH 2 to form a precipitate, then the liquid was extracted with EtOAc (30 mL). The organic layer was separated, dried over anhydrous Na₂SO₄, and concentrated in vacuo. The residue was dissolved in hot EtOAc (5 mL) and PE (10 mL) was added to form a light yellow precipitate, which was collected and dried in vacuo to give 2-[2-[4-(8-chloro-4-methyl-quinazolin-2-yl)phenoxy]ethoxy]acetic acid (Example 11, 271 mg). ¹H NMR (DMSO-d₆, 400 MHz)): δ ppm 12.67 (br s, 1H), 8.50-8.59 (m, 2H), 8.25 (dd, J=8.31, 1.22 Hz, 1H), 8.14 (dd, J=7.52, 1.16 Hz, 1H), 7.57-7.67 (m, 1H), 7.15 (d, J=9.05 Hz, 2H), 4.23 (dd, J=5.44, 3.61 Hz, 2H), 4.13 (s, 2H), 3.82-3.91 (m, 2H), 3.00 (s, 3H). MS obsd. (ESI⁺) [(M+H)⁺]: 374.1.

### Example 12

### cis-3-[2-[4-(8-Chloro-4-methyl-quinazolin-2-yl)phenoxy]ethoxy]cyclobutanecarboxylic acid

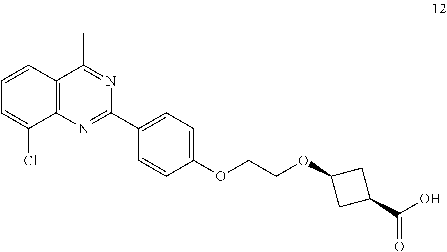 [SMILES: Cc1nc(-c2ccc(OCCO[C@H]3C[C@@H](C(=O)O)C3)cc2)nc2c(Cl)cccc12]

### Step 1: Preparation of cis-ethyl 3-[2-[4-(8-chloro-4-methyl-quinazolin-2-yl)phenoxy]ethoxy]cyclobutanecarboxylate

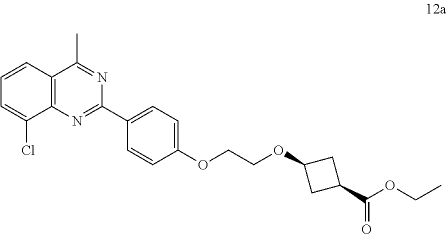 [SMILES: CCOC(=O)[C@H]1C[C@@H](OCCOc2ccc(-c3nc(C)c4cccc(Cl)c4n3)cc2)C1]

Compound 12a was prepared in analogy to example 10 by using cis-ethyl 3-hydroxycyclobutanecarboxylate (137 mg, 0.953 mmol) instead of methyl 2-hydroxyacetate. cis-Ethyl 3-[2-[4-(8-chloro-4-methyl-quinazolin-2-yl)phenoxy]ethoxy]cyclobutanecarboxylate (compound 12a, 140 mg) was obtained as a light yellow solid. MS obsd. (ESI⁺) [(M+H)⁺]: 441.2.

### Step 2: Preparation of cis-3-[2-[4-(8-chloro-4-methyl-quinazolin-2-yl)phenoxy]ethoxy]cyclobutanecarboxylic acid

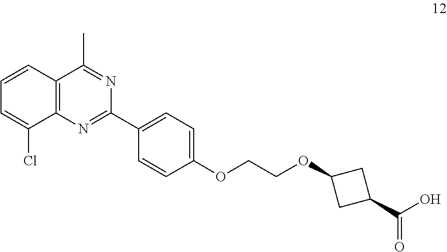 [SMILES: Cc1nc(-c2ccc(OCCO[C@H]3C[C@@H](C(=O)O)C3)cc2)nc2c(Cl)cccc12]

Example 12 was prepared in analogy to Example 11 by using cis-ethyl 3-[2-[4-(8-chloro-4-methyl-quinazolin-2-yl)phenoxy]ethoxy]cyclobutanecarboxylate (compound 12a, 130 mg, 0.295 mmol) instead of methyl 2-[2-[4-(8-chloro-4-methyl-quinazolin-2-yl)phenoxy]ethoxy]acetate (Example 10). cis-3-[2-[4-(8-Chloro-4-methyl-quinazolin-2-yl)phenoxy]ethoxy]cyclobutanecarboxylic acid (Example 12, 95 mg) was obtained as a white solid. ¹H NMR (DMSO-d₆, 400 MHz)): δ ppm 12.15 (br s, 1H), 8.55 (d, J=8.93 Hz, 2H), 8.24 (dd, J=8.25, 1.16 Hz, 1H), 8.13 (dd, J=7.52, 1.16 Hz, 1H), 7.58-7.67 (m, 1H), 7.14 (d, J=8.93 Hz, 2H), 4.14-4.23 (m, 2H), 3.90-4.02 (m, 1H), 3.63-3.72 (m, 2H), 3.00 (s, 3H), 2.54-2.64 (m, 1H), 2.41-2.49 (m, 2H), 1.96-2.06 (m, 2H). MS obsd. (ESI⁺) [(M+H)⁺]: 413.1.

### Example 13

### 8-Chloro-2-(4-chlorophenyl)-4-methylquinazoline

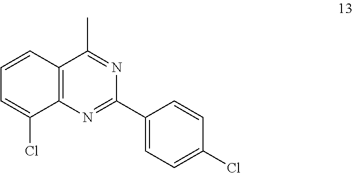 [SMILES: Cc1nc(-c2ccc(Cl)cc2)nc2c(Cl)cccc12]

To a solution of 2,8-dichloro-4-methyl-quinazoline (compound 9d, 200 mg, 0.939 mmol) and (4-chlorophenyl)boronic acid (147 mg, 0.939 mmol) in 1,4-Dioxane (6 mL) were added 2N aqueous K₂CO₃ (1.5 mL) and Pd(Ph₃P)₄ (86.8 mg, 0.075 mmol) at rt, the mixture was stirred at 110° C. for 3 hrs. After being cooled to rt, the reaction mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:10 to 100:50 (v/v) to give 8-chloro-2-(4-chlorophenyl)-4-methylquinazoline (Example 13, 200 mg) as a solid. ¹H NMR (400 MHz, DMSO-d₆) δ ppm 8.63-8.70 (m, 2H), 8.21 (dd, J=0.98, 8.31 Hz, 1H), 8.06 (dd, J=0.98, 7.58 Hz, 1H), 7.62 (t, J=7.95 Hz, 1H), 7.51-7.58 (m, 2H), 3.05 (s, 3H). MS obsd. (ESI⁺) [(M+H)⁺]: 289.0.

### Example 14

### 2-(2-(4-(4-Methyl-7-(trifluoromethyl)quinazolin-2-yl)phenoxy)ethoxy)acetic acid

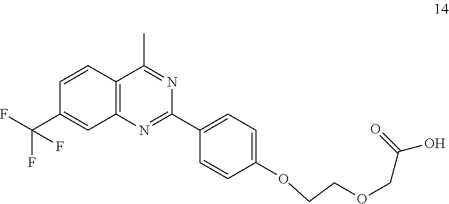 [SMILES: Cc1nc(-c2ccc(OCCOCC(=O)O)cc2)nc2cc(C(F)(F)F)ccc12]

### Step 1: Preparation of ethyl 2-(2-(tosyloxy)ethoxy)acetate

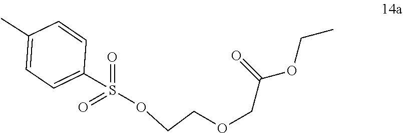 [SMILES: CCOC(=O)COCCOS(=O)(=O)c1ccc(C)cc1]

To a solution of ethyl 2-(2-hydroxyethoxy)acetate (6 g, 40.5 mmol) and N,N-dimethylpyridin-4-amine (4.95 g, 40.5 mmol) in DCM (80 mL) was added 4-methylbenzene-1-sulfonyl chloride (8.49 g, 44.5 mmol) at 0° C., then the mixture was warmed to rt for 48 hrs. After the reaction was completed, the mixture was partitioned between DCM (200 mL) and water (100 mL), and the separated organic layer was washed with 1N aqueous HCl (50 mL), saturated NaHCO₃ (50 mL) solution, and brine (30 mL). The organic phase was dried over Na₂SO₄ and concentrated in vacuo to give the crude ethyl 2-(2-(tosyloxy)ethoxy)acetate (compound 14a, 6 g, 49%) as a colorless oil. MS obsd. (ESI⁺) [(M+H)⁺]: 303.1

### Step 2: Preparation of N-(2-acetyl-5-(trifluoromethyl)phenyl)-4-methoxybenzamide

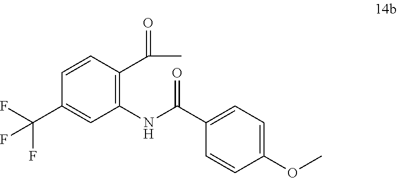 [SMILES: COc1ccc(C(=O)Nc2cc(C(F)(F)F)ccc2C(C)=O)cc1]

To a stirred solution of 1-(2-amino-4-(trifluoromethyl)phenyl)ethanone (6.01 g, 29.5 mmol) in DCM (10 mL) was added 4-methoxy-benzyl chloride (5.03 g, 29.5 mmol) and 4-methylmorpholine (2.99 g, 29.5 mmol) at 0° C., then the mixture was stirred at rt overnight. The mixture was partitioned between DCM (100 mL) and water (100 mL), and the separated aqueous phase was extracted with DCM (100 mL) twice. The combined organic phase was dried over Na₂SO₄ and concentrated in vacuo. The residue was purified by column chromatography on silica gel using gradient elution with PE-EtOAc from 100:10 to 100:50 (v/v) to give N-(2-acetyl-5-(trifluoromethyl)phenyl)-4-methoxybenzamide (compound 14b, 7 g) as a light yellow solid. MS obsd. (ESI⁺) [(M+H)⁺]: 338.1

### Step 3: Preparation of 2-(4-methoxyphenyl)-4-methyl-7-(trifluoromethyl)quinazoline

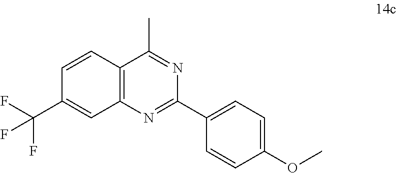 [SMILES: COc1ccc(-c2nc(C)c3ccc(C(F)(F)F)cc3n2)cc1]

A mixture of N-(2-acetyl-5-(trifluoromethyl)phenyl)-4-methoxybenzamide (compound 14b, 10 g, 29.6 mmol) and ammonium acetate (11.4 g, 148 mmol) in EtOH (60 mL) was stirred at 80° C. for 1 hr. After being cooled to rt, the mixture was partitioned between EtOAc (100 mL) and water (100 mL), and the separated aqueous phase was extracted with EtOAc (100 mL) twice. The combined organic phase was dried over Na₂SO₄ and concentrated in vacuo. The residue was purified by column chromatography on silica gel using gradient elution with PE-EtOAc from 100:10 to 100:50 (v/v) to give 2-(4-methoxyphenyl)-4-methyl-7-(trifluoromethyl)quinazoline (compound 14c, 8 g) as a light yellow foam. MS obsd. (ESI⁺) [(M+H)⁺]: 319.1

### Step 4: Preparation of 4-(4-methyl-7-(trifluoromethyl)quinazolin-2-yl)phenol

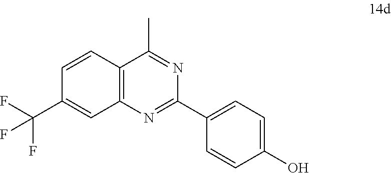 [SMILES: Cc1nc(-c2ccc(O)cc2)nc2cc(C(F)(F)F)ccc12]

To a solution of 2-(4-methoxyphenyl)-4-methyl-7-(trifluoromethyl)quinazoline (compound 14c, 4 g, 12.6 mmol) in DCM (20 mL) was added 3.0 M tribromoborane solution in DCM (16.8 mL, 50.3 mmol). After the reaction mixture was stirred at rt overnight, it was poured into ice-water (100 mL) carefully. The solution was partitioned between EtOAc (100 mL) and water (100 mL), and the separated aqueous phase was extracted with EtOAc (100 mL) twice. The combined organic phase was dried over Na₂SO₄ and concentrated in vacuo to give the crude 4-(4-methyl-7-(trifluoromethyl)quinazolin-2-yl)phenol (compound 14d, 3 g) as a yellow solid. MS obsd. (ESI⁺) [(M+H)⁺]: 305.1

### Step 5: Preparation of ethyl 2-(2-(4-(4-methyl-7-(trifluoromethyl)quinazolin-2-yl)phenoxy)ethoxy)acetate

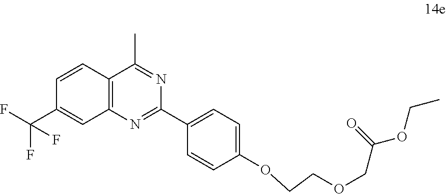 [SMILES: CCOC(=O)COCCOc1ccc(-c2nc(C)c3ccc(C(F)(F)F)cc3n2)cc1]

To a solution of 4-(4-methyl-7-(trifluoromethyl)quinazolin-2-yl)phenol (compound 14d, 400 mg, 1.31 mmol), ethyl 2-(2-(tosyloxy)ethoxy)acetate (compound 14a, 437 mg, 1.45 mmol) in DMF (5 mL) was added K₂CO₃ (363 mg, 2.63 mmol), and the mixture was heated to 50° C. overnight. After being cooled to rt, the mixture was partitioned between EtOAc (20 mL) and water (20 mL), and the separated aqueous phase was extracted with EtOAc (10 mL) twice. The combined organic phase was dried over Na₂SO₄ and concentrated in vacuo to give the crude ethyl 2-(2-(4-(4-methyl-7-(trifluoromethyl)quinazolin-2-yl)phenoxy)ethoxy)acetate (compound 14e, 450 mg, 1.04 mmol) as a yellow solid. MS obsd. (ESI⁺) [(M+H)⁺]: 435.1

### Step 6: Preparation of 2-(2-(4-(4-methyl-7-(trifluoromethyl)quinazolin-2-yl)phenoxy)ethoxy)acetic acid

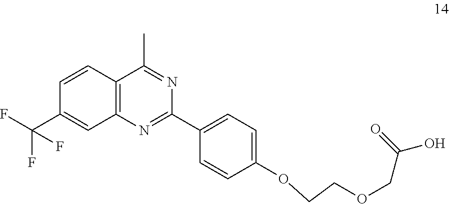 [SMILES: Cc1nc(-c2ccc(OCCOCC(=O)O)cc2)nc2cc(C(F)(F)F)ccc12]

To a solution of ethyl 2-(2-(4-(4-methyl-7-(trifluoromethyl)quinazolin-2-yl)phenoxy)ethoxy)acetate (compound 14e, 400 mg, 0.921 mmol) in THF (4 mL) was added 3.0 N aqueous HCl (4 mL, 12 mmol), and the mixture was heated to 50° C. for 2 hrs. After being cooled to rt, the mixture was concentrated in vacuo. The residue was purified by preparative HPLC to give 2-(2-(4-(4-methyl-7-(trifluoromethyl)quinazolin-2-yl)phenoxy)ethoxy)acetic acid (Example 14, 150 mg) as a light yellow foam. ¹H NMR (400 MHz, DMSO-d₆) δ ppm 12.01-12.24 (m, 1H), 8.47-8.56 (m, 3H), 8.30-8.35 (m, 1H), 7.89-7.95 (m, 1H), 7.11-7.19 (m, 2H), 4.20-4.26 (m, 2H), 4.09 (s, 2H), 3.82-3.90 (m, 2H), 3.04 (s, 3H). MS obsd. (ESI⁺) [(M+H)⁺]: 407.1

### Example 15

### 3-(2-(4-(4-Methyl-7-(trifluoromethyl)quinazolin-2-yl)phenoxy)ethoxy) cyclobutanecarboxylic acid

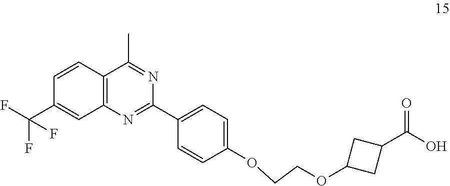 [SMILES: Cc1nc(-c2ccc(OCCOC3CC(C(=O)O)C3)cc2)nc2cc(C(F)(F)F)ccc12]

### Step 1: Preparation of 2-benzyloxyethoxy(trimethyl)silane

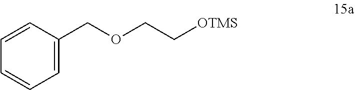

To a solution of 2-benzyloxyethanol (20.0 g, 131.4 mmol) and TEA (20.0 g, 197.1 mmol) in DCM (200 mL) at 0° C. was added trimethylsilyl chloride (17.1 g, 157.7 mmol) and the mixture was then stirred at 25° C. for 16 hrs. After the reaction was completed, the mixture was concentrated in vacuo and the residue was purified by column chromatography on silica gel using gradient elution with PE-EtOAc from 50:1 to 10:2 (v/v) to give the 2-benzyloxyethoxy(trimethyl)silane (compound 15a, 25.0 g, 84.9%) as a colorless oil.

### Step 2: Preparation of methyl 3-(2-benzyloxyethoxy)cyclobutanecarboxylate

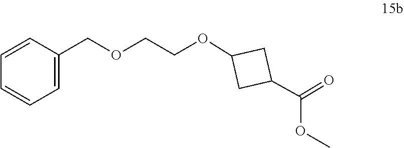 [SMILES: COC(=O)C1CC(OCCOCc2ccccc2)C1]

To a solution of 2-benzyloxyethoxy(trimethyl)silane (25.0 g, 111.4 mmol) and methyl 3-oxocyclobutanecarboxylate (CAS #: 4934-99-0, Cat. #: PB01390, from PharmaBlock (Nanjing) R&D Co. Ltd, 15.0 g, 117.0 mmol) in DCM (200 mL) was added trimethylsilyl trifluoromethanesulfonate (12.4 g, 55.7 mmol) dropwise at −78° C. After addition, the mixture was stirred at −78° C. for additional 1 hr, and then to the resulting mixture was added triethylsilane (14.25 g, 122.57 mmol). After addition, the resulting mixture was warmed to room temperature and stirred for 1 hr. After the reaction was completed, the mixture was washed with saturated NH₄Cl solution and brine, dried over anhydrous sodium sulfate, and concentrated in vacuo. The residue was purified by column chromatography on silica gel using gradient elution with PE-EtOAc from 100:1 to 50:1 (v/v) to give methyl 3-(2-benzyloxyethoxy)cyclobutanecarboxylate (compound 15b, 28 g, 95.1%) as a colorless oil. MS obsd. (ESI⁺) [(M+H)⁺]: 265.1.

### Step 3: Preparation of methyl 3-(2-hydroxyethoxy)cyclobutanecarboxylate

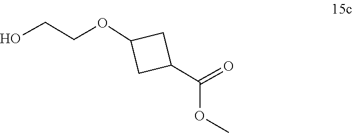

To a solution of methyl 3-(2-benzyloxyethoxy)cyclobutanecarboxylate (compound 15b, 28.0 g, 105.9 mmol) in MeOH (300.0 mL) was added Pd(OH)₂ (wet) (1.48 g, 10.6 mmol) at room temperature and the mixture was then hydrogenated under H₂ atmosphere at room temperature overnight. After the reaction was completed, the mixture was filtered through silica gel pad and the filtrate was concentrated in vacuo to give crude methyl 3-(2-hydroxyethoxy)cyclobutanecarboxylate (compound 15c, 18 g, 97.6%) as a colorless oil.

### Step 4: methyl 3-[2-(p-tolylsulfonyloxy)ethoxy]cyclobutanecarboxylate

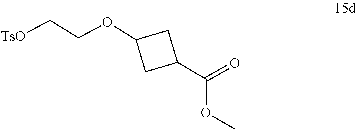 [SMILES: COC(=O)C1CC(OCCOS(=O)(=O)c2ccc(C)cc2)C1]

To a solution of methyl 3-(2-hydroxyethoxy)cyclobutanecarboxylate (compound 15c, 5 g, 28.7 mmol) and DMAP (5.26 g, 43.1 mmol) in DCM (80 mL) was added 4-methylbenzene-1-sulfonyl chloride (6.02 g, 31.6 mmol) at room temperature and the mixture was then stirred at room temperature overnight. After the reaction was completed, the reaction mixture was washed with 1N aqueous HCl (25 mL), water (15 mL), saturated NaHCO₃ solution and brine, and then concentrated in vacuo to give the crude methyl 3-[2-(p-tolylsulfonyloxy)ethoxy]cyclobutanecarboxylate (compound 15d, 8.1 g, 85.6%) as a colorless oil, which was used in next step directly without further purification. MS obsd. (ESI⁺) [(M+H)⁺]: 329.2.

### Step 5: Preparation of methyl 3-(2-(4-(4-methyl-7-(trifluoromethyl)quinazolin-2-yl)phenoxy)ethoxy)cyclobutanecarboxylate

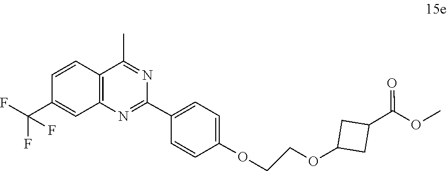 [SMILES: COC(=O)C1CC(OCCOc2ccc(-c3nc(C)c4ccc(C(F)(F)F)cc4n3)cc2)C1]

Compound 15e was prepared in analogy to Example 14, Step 5 by using methyl 3-[2-(p-tolylsulfonyloxy)ethoxy]cyclobutanecarboxylate (compound 15d, 475 mg, 1.45 mmol) instead of ethyl 2-(2-(tosyloxy)ethoxy)acetate (compound 14a). Methyl 3-(2-(4-(4-methyl-7-(trifluoromethyl)quinazolin-2-yl)phenoxy)ethoxy)cyclobutanecarboxylate (compound 15e, 400 mg, 66.1%) was obtained as a yellow solid. MS obsd. (ESI⁺) [(M+H)⁺]: 461.1 Step 6: Preparation of 3-(2-(4-(4-methyl-7-(trifluoromethyl)quinazolin-2-yl)phenoxy)ethoxy)cyclobutanecarboxylic acid

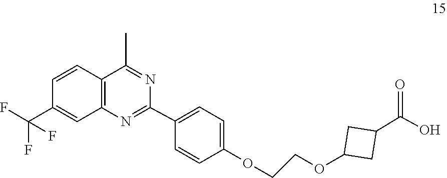 [SMILES: Cc1nc(-c2ccc(OCCOC3CC(C(=O)O)C3)cc2)nc2cc(C(F)(F)F)ccc12]

Example 15 was prepared in analogy to Example 14, Step 6 by using methyl 3-(2-(4-(4-methyl-7-(trifluoromethyl)quinazolin-2-yl)phenoxy)ethoxy)cyclobutanecarboxylate (compound 15e, 475 mg, 1.45 mmol) instead of ethyl 2-(2-(4-(4-methyl-7-(trifluoromethyl)quinazolin-2-yl)phenoxy)ethoxy) acetate (compound 14e). 3-(2-(4-(4-Methyl-7-(trifluoromethyl)quinazolin-2-yl)phenoxy)ethoxy)cyclobutanecarboxylic acid (Example 15, 42 mg) was obtained as a light yellow foam ¹H NMR (400 MHz, DMSO-d₆) δ ppm 12.05-12.44 (m, 1H), 8.41-8.55 (m, 3H), 8.29-8.36 (m, 1H), 7.88-7.95 (m, 1H), 7.08-7.17 (m, 2H), 4.14-4.22 (m, 2H), 3.91-4.01 (m, 1H), 3.64-3.72 (m, 2H), 3.03 (s, 3H), 2.87-2.97 (m, 1H), 2.36-2.48 (m, 1H), 2.11-2.22 (m, 2H), 1.99-2.06 (m, 1H). MS obsd. (ESI⁺) [(M+H)⁺]: 447.1

### Example 16

### 2-(3-Bromophenyl)-4-(methoxymethyl)-7-(trifluoromethyl)quinazoline

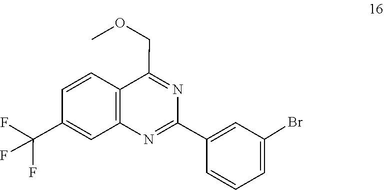 [SMILES: COCc1nc(-c2cccc(Br)c2)nc2cc(C(F)(F)F)ccc12]

### Step 1: Preparation of N-(2-acetyl-5-(trifluoromethyl)phenyl)-3-bromobenzamide

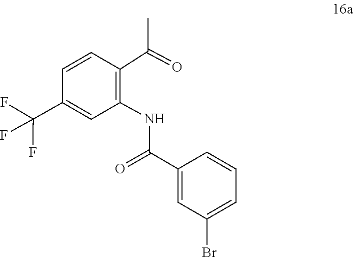 [SMILES: CC(=O)c1ccc(C(F)(F)F)cc1NC(=O)c1cccc(Br)c1]

Compound 16a was prepared in analogy to Example 14, step 2 by using 3-bromobenzoyl chloride (1.3 g, 5.91 mmol) instead of 4-methoxy-benzyl chloride. N-(2-acetyl-5-(trifluoromethyl)phenyl)-3-bromobenzamide (compound 16a, 1.7 g) to give a solid. MS obsd. (ESI⁺) [(M+H)⁺]: 386.0.

### Step 2: Preparation of 2-(3-bromophenyl)-4-methyl-7-(trifluoromethyl)quinazoline

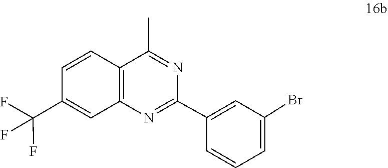 [SMILES: Cc1nc(-c2cccc(Br)c2)nc2cc(C(F)(F)F)ccc12]

Compound 16b was prepared in analogy to Example 14, Step 3 by using N-(2-acetyl-5-(trifluoromethyl)phenyl)-3-bromobenzamide (compound 16a, 1.7 g, 4.4 mmol) instead of N-(2-acetyl-5-(trifluoromethyl)phenyl)-4-methoxybenzamide (compound 14b). 2-(3-bromophenyl)-4-methyl-7-(trifluoromethyl)quinazoline (compound 16b, 1.4 g, 86.6%) was obtained as a solid. MS obsd. (ESI⁺) [(M+H)⁺]: 367.0.

### Step 3: Preparation of 4-(bromomethyl)-2-(3-bromophenyl)-7-(trifluoromethyl)quinazoline

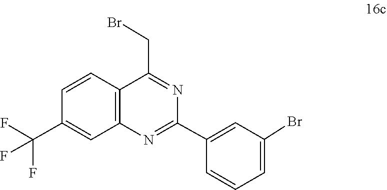 [SMILES: FC(F)(F)c1ccc2c(CBr)nc(-c3cccc(Br)c3)nc2c1]

To a solution of 2-(3-bromophenyl)-4-methyl-7-(trifluoromethyl)quinazoline (compound 16 b, 400 mg, 1.09 mmol) in EtOAc (20 mL) was added copper (II) bromide (511 mg, 2.29 mmol) at rt, then the mixture was stirred at 85° C. overnight. After the reaction was completed, the mixture was filtered and the filtrate was concentrated in vacuo. The residue was purified by column chromatography on silica gel using gradient elution with PE-EtOAc from 100:0 to 100:30 (v/v) to give 4-(bromomethyl)-2-(3-bromophenyl)-7-(trifluoromethyl)quinazoline (compound 16c, 180 mg, 37%) as a solid. MS obsd. (ESI⁺) [(M+H)⁺]: 444.9.

### Step 3: Preparation of 2-(3-bromophenyl)-4-(methoxymethyl)-7-(trifluoromethyl)quinazoline

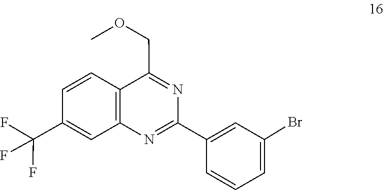 [SMILES: COCc1nc(-c2cccc(Br)c2)nc2cc(C(F)(F)F)ccc12]

To a solution of 4-(bromomethyl)-2-(3-bromophenyl)-7-(trifluoromethyl)quinazoline (compound 16c, 50 mg, 0.112 mmol) in MeOH (2 mL) was added sodium methoxide (6 mg, 0.112 mmol) at rt, and the mixture was stirred overnight. After the reaction was completed, the mixture was neutralized with AcOH. The solvent was removed in vacuo. The residue was purified by preparative HPLC to give 2-(3-bromophenyl)-4-(methoxymethyl)-7-(trifluoromethyl)quinazoline (compound 16, 3.5 mg) as a solid. ¹H NMR (400 MHz, DMSO-d₆) δ ppm 8.74 (t, J=1.76 Hz, 1H), 8.61 (t, J=9.29 Hz, 2H), 8.51 (s, 1H), 8.05 (dd, J=1.76, 8.78 Hz, 1H), 7.80-7.86 (m, 1H), 7.57-7.66 (m, 1H), 5.18 (s, 2H), 3.48 (s, 3H). MS obsd. (ESI⁺) [(M+H)⁺]: 397.0.

### Example 17

### 1-(2-(3-Bromophenyl)-7-(trifluoromethyl)quinazolin-4-yl)-N,N-dimethylmethanamine

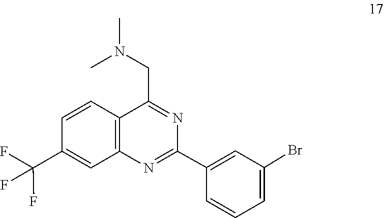 [SMILES: CN(C)Cc1nc(-c2cccc(Br)c2)nc2cc(C(F)(F)F)ccc12]

To a solution of 4-(bromomethyl)-2-(3-bromophenyl)-7-(trifluoromethyl)quinazoline (compound 16c, 50 mg, 0.112 mmol) in acetonitrile (2 mL) was added dimethylamine (0.112 ml, 0.224 mmol) at rt. After the reaction was completed, the mixture was neutralized with AcOH. The solvent was removed in vacuo. The residue was purified by preparative HPLC to give 1-(2-(3-bromophenyl)-7-(trifluoromethyl)quinazolin-4-yl)-N,N-dimethylmethanamine (Example 17, 4 mg, 8.35%) as a solid. ¹H NMR (400 MHz, DMSO-d₆) δ ppm 8.81 (d, J=8.78 Hz, 1H), 8.72 (t, J=1.63 Hz, 1H), 8.59 (d, J=8.03 Hz, 1H), 8.47 (s, 1H), 8.02 (dd, J=1.76, 8.78 Hz, 1H), 7.79-7.85 (m, 1H), 7.59 (s, 1H), 4.15 (s, 2H), 2.31 (s, 6H). MS obsd. (ESI⁺) [(M+H)⁺]: 410.0.

### Example 18

### 4-((2-(3-Bromophenyl)-7-(trifluoromethyl)quinazolin-4-yl)methyl)morpholine

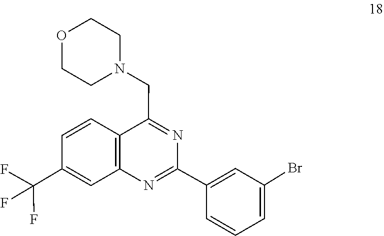 [SMILES: FC(F)(F)c1ccc2c(CN3CCOCC3)nc(-c3cccc(Br)c3)nc2c1]

Example 18 was prepared in analogy to Example 17 by using morpholine instead of dimethylamine. 4-((2-(3-Bromophenyl)-7-(trifluoromethyl)quinazolin-4-yl)methyl)morpholine (Example 18, 14.5 mg) was obtained as a solid. ¹H NMR (400 MHz, DMSO-d₆) δ ppm 8.84 (d, J=8.78 Hz, 1H), 8.72 (t, J=1.76 Hz, 1H), 8.55-8.60 (m, 1H), 8.47 (s, 1H), 8.03 (dd, J=1.76, 8.78 Hz, 1H), 7.82 (ddd, J=0.88, 2.07, 7.84 Hz, 1H), 7.59 (t, J=7.91 Hz, 1H), 4.23 (s, 2H), 3.52-3.61 (m, 4H), 2.58 (d, J=4.27 Hz, 4H). MS obsd. (ESI⁺) [(M+H)⁺]: 452.1.

### Example 19

### 2-(2-(3-Bromobenzamido)-4-(trifluoromethyl)phenyl)-2-oxoethyl acetate

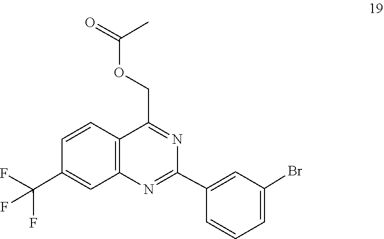 [SMILES: CC(=O)OCc1nc(-c2cccc(Br)c2)nc2cc(C(F)(F)F)ccc12]

### Step 1: Preparation of 3-bromo-N-(2-(2-bromoacetyl)-5-(trifluoromethyl)phenyl)benzamide

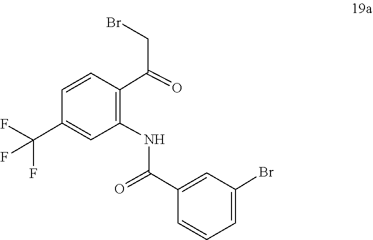 [SMILES: O=C(Nc1cc(C(F)(F)F)ccc1C(=O)CBr)c1cccc(Br)c1]

Compound 19a was prepared in analogy to Example 16, step 3 by using N-[2-acetyl-5-(trifluoromethyl)phenyl]-3-bromo-benzamide (compound 16a) (1.2 g, 3.11 mmol) instead of 2-(3-bromophenyl)-4-methyl-7-(trifluoromethyl)quinazoline (compound 16b) to give 3-bromo-N-(2-(2-bromoacetyl)-5-(trifluoromethyl)phenyl)benzamide (compound 19a, 1.4 g, 96.9%) as a solid. MS obsd. (ESI⁺) [(M+H)⁺]: 463.9.

### Step 2: Preparation of 2-(2-(3-bromobenzamido)-4-(trifluoromethyl)phenyl)-2-oxoethyl acetate

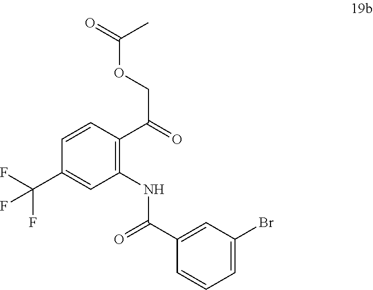 [SMILES: CC(=O)OCC(=O)c1ccc(C(F)(F)F)cc1NC(=O)c1cccc(Br)c1]

To a solution of 3-bromo-N-(2-(2-bromoacetyl)-5-(trifluoromethyl)phenyl)benzamide (compound 19a, 450 mg, 0.968 mmol) in acetonitrile (5 mL) was added potassium acetate (190 mg, 1.94 mmol) at rt, then the mixture was stirred at 60° C. for 1.5 hrs. After the reaction was completed, the solvent was removed in vacuo. The residual was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:0 to 100:100 (v/v) to give the 2-(2-(3-bromobenzamido)-4-(trifluoromethyl)phenyl)-2-oxoethyl acetate (compound 19b, 200 mg, 41.9%) as solid. MS obsd. (ESI⁺) [(M+H)⁺]: 444.0

### Step 3: Preparation of 2-(2-(3-bromobenzamido)-4-(trifluoromethyl)phenyl)-2-oxoethyl acetate

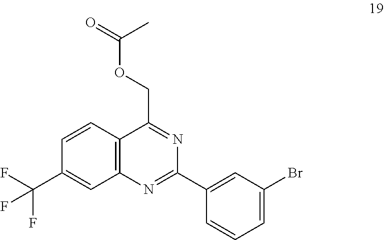 [SMILES: CC(=O)OCc1nc(-c2cccc(Br)c2)nc2cc(C(F)(F)F)ccc12]

[2-(3-Bromophenyl)-7-(trifluoromethyl)quinazolin-4-yl]methyl acetate was prepared in analogy to Example 14, step 3 by using 2-(2-(3-bromobenzamido)-4-(trifluoromethyl)phenyl)-2-oxoethyl acetate (compound 19b, 200 mg, 0.45 mmol) instead of N-(2-acetyl-5-(trifluoromethyl)phenyl)-4-methoxybenzamide (compound 14b). 2-(2-(3-Bromobenzamido)-4-(trifluoromethyl)phenyl)-2-oxoethyl acetate give a solid (Example 19, 9 mg, 4.7%) was obtained as a solid. ¹H NMR (400 MHz, DMSO-d₆) δ ppm 8.70 (t, J=1.63 Hz, 1H), 8.52-8.59 (m, 3H), 8.05-8.10 (m, 1H), 7.83 (d, J=7.78 Hz, 1H), 7.61 (t, J=7.91 Hz, 1H), 5.93 (s, 2H), 2.28 (s, 3H). MS obsd. (ESI⁺) [(M+H)⁺]: 425.0.

### Example 20

### (2-(3-Bromophenyl)-7-(trifluoromethyl)quinazolin-4-yl)methanol

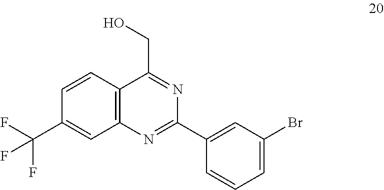 [SMILES: OCc1nc(-c2cccc(Br)c2)nc2cc(C(F)(F)F)ccc12]

To a solution of [2-(3-bromophenyl)-7-(trifluoromethyl)quinazolin-4-yl]methyl acetate (Example 19, 100 mg, 0.235 mmol) in MeOH (2 mL) was added K₂CO₃ (32.5 mg, 0.235 mmol) at rt for 10 mins. After the reaction was completed, the mixture was neutralized with AcOH and concentrated in vacuo. The residue was partitioned between EtOAc (10 mL) and water (10 mL). The separated aqueous phase was extracted with EtOAc (10 mL) twice. The combined organic phase was dried over Na₂SO₄ and concentrated in vacuo. The residue was purified by preparative HPLC to give (2-(3-bromophenyl)-7-(trifluoromethyl)quinazolin-4-yl)methanol (Example 20, 2.4 mg) as a solid. ¹H NMR (400 MHz, DMSO-d₆) δ ppm 8.82 (s, 1H), 8.66 (t, J=7.78 Hz, 2H), 8.49 (s, 1H), 8.01-8.06 (m, 1H), 7.82 (d, J=7.03 Hz, 1H), 7.60 (s, 1H), 5.88 (t, J=6.02 Hz, 1H), 5.23 (d, J=5.77 Hz, 2H). MS obsd. (ESI⁺) [(M+H)⁺]: 383.0.

### Example 21

### (8-Chloro-2-(4-chlorophenyl)quinazolin-4-yl)methanol

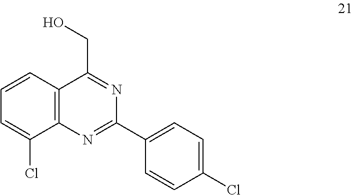 [SMILES: OCc1nc(-c2ccc(Cl)cc2)nc2c(Cl)cccc12]

### Step 1: Preparation of 4-(bromomethyl)-8-chloro-2-(4-chlorophenyl)quinazoline

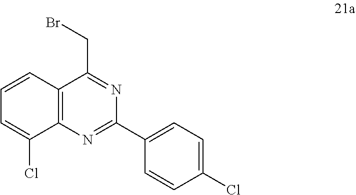 [SMILES: Clc1ccc(-c2nc(CBr)c3cccc(Cl)c3n2)cc1]

To a solution of 8-chloro-2-(4-chlorophenyl)-4-methylquinazoline (Example 13, 170 mg, 0.588 mmol) in EtOAc (5 mL) was added copper (II) bromide (144 mg, 0.647 mmol) at rt, then the mixture was stirred at 85° C. overnight. After the reaction was completed, the mixture was filtered and the filtrate was concentrated in vacuo to give the crude 4-(bromomethyl)-8-chloro-2-(4-chlorophenyl)quinazoline (compound 21a, 170 mg, 462 μmol) as a solid. MS obsd. (ESI⁺) [(M+H)⁺]: 366.9.

### Step 2: Preparation of (8-chloro-2-(4-chlorophenyl)quinazolin-4-yl)methanol

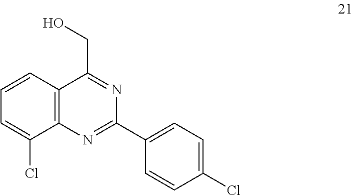 [SMILES: OCc1nc(-c2ccc(Cl)cc2)nc2c(Cl)cccc12]

To a solution of 4-(bromomethyl)-8-chloro-2-(4-chlorophenyl)quinazoline (compound 21a, 170 mg, 0.462 mmol) in DMF (3 mL) was added sodium acetate (75.8 mg, 0.924 mmol) at rt and the mixture was stirred at 80° C. for 30 mins. After being cooled to rt, aqueous KOH (1N, 0.5 mL) was added to the mixture and the mixture was stirred at rt for 30 mins. The reaction mixture was purified by preparative HPLC to give (8-chloro-2-(4-chlorophenyl)quinazolin-4-yl)methanol (Example 21, 12 mg, 8.26%) as a solid. ¹H NMR (400 MHz, METHANOL-d₄) δ ppm 8.70-8.80 (m, 2H), 8.27 (dd, J=1.22, 8.31 Hz, 1H), 8.10 (dd, J=1.22, 7.58 Hz, 1H), 7.61-7.70 (m, 1H), 7.53-7.60 (m, 2H), 5.32 (s, 2H). MS obsd. (ESI⁺) [(M+H)⁺]: 305.0.

### Example 22

### (3-(4-Methyl-7-(trifluoromethyl)quinazolin-2-yl)phenyl)methanol

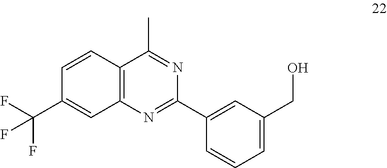 [SMILES: Cc1nc(-c2cccc(CO)c2)nc2cc(C(F)(F)F)ccc12]

### Preparation of (3-(4-methyl-7-(trifluoromethyl)quinazolin-2-yl)phenyl)methanol

To a solution of 2-chloro-4-methyl-7-(trifluoromethyl)quinazoline (80 mg, 0.324 mmol) and Pd(Ph₃P)₄ (30 mg, 0.026 mmol) in 1,4-dioxane (2 mL) and 2 N K₂CO₃ solution (0.5 mL) was added (3-(hydroxymethyl)phenyl)boronic acid (73.9 mg, 0.487 mmol), then the mixture was heated to 110° C. overnight. After the reaction was completed, the mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residual was purified by preparative HPLC to give (3-(4-methyl-7-(trifluoromethyl)quinazolin-2-yl)phenyl)methanol (Example 22, 17 mg, 16.3%) as a solid. ¹H NMR (400 MHz, DMSO-d6) δ ppm 8.58 (s, 1H), 8.53 (d, J=8.78 Hz, 1H), 8.46 (td, J=1.88, 6.78 Hz, 1H), 8.39 (s, 1H), 7.97 (dd, J=1.76, 8.53 Hz, 1H), 7.49-7.58 (m, 2H), 5.34-5.41 (m, 1H), 4.64 (d, J=5.77 Hz, 2H), 3.06 (s, 3H). MS obsd. (ESI⁺) [(M+H)⁺]: 319.1.

### Example 23

### 8-Chloro-2-(6-chloropyridin-3-yl)-4-methylquinazoline

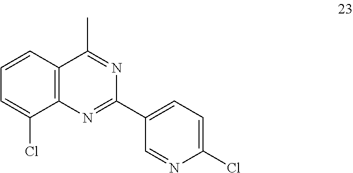 [SMILES: Cc1nc(-c2ccc(Cl)nc2)nc2c(Cl)cccc12]

Example 23 was prepared in analogy to Example 8 by using 6-chloronicotinimidamide hydrochloride instead of 4-hydroxybenzimidamide hydrochloride. 8-Chloro-2-(6-chloropyridin-3-yl)-4-methylquinazoline (Example 23, 20 mg) was obtained as a solid. ¹H NMR (400 MHz, DMSO-d₆) δ ppm 9.48-9.51 (m, 1H), 8.90 (d, J=2.45 Hz, 1H), 8.88 (d, J=2.45 Hz, 1H), 8.34 (dd, J=1.22, 8.31 Hz, 1H), 8.23 (dd, J=1.22, 7.58 Hz, 1H), 7.71-7.79 (m, 2H), 3.06 (s, 3H). MS obsd. (ESI⁺) [(M+H)⁺]: 290.0.

### Example 24

### 8-Chloro-2-(6-chloropyridin-3-yl)quinazoline

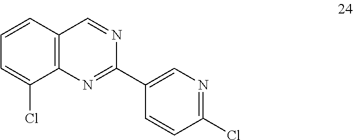 [SMILES: Clc1ccc(-c2ncc3cccc(Cl)c3n2)cn1]

Example 24 was prepared in analogy to Example 1 by using 6-chloronicotinimidamide hydrochloride (185 mg, 0.964 mmol) instead of 4-bromobenzimidamide hydrochloride. 8-Chloro-2-(6-chloropyridin-3-yl)quinazoline (Example 24, 130 mg) was obtained as a solid. ¹H NMR (400 MHz, DMSO-d₆) δ ppm 9.84 (s, 1H), 9.51 (d, J=2.20 Hz, 1H), 8.90 (dd, J=2.45, 8.31 Hz, 1H), 8.25 (ddd, J=1.10, 7.83, 18.95 Hz, 2H), 7.71-7.83 (m, 2H). MS obsd. (ESI⁺) [(M+H)⁺]E: 276.0.

### Example 25

### 5-(8-Chloroquinazolin-2-yl)pyridin-2-ol

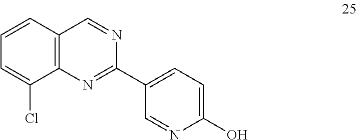 [SMILES: Oc1ccc(-c2ncc3cccc(Cl)c3n2)cn1]

8-Chloro-2-(6-chloropyridin-3-yl)quinazoline (Example 24, 25 mg, 0.091 mmol) in the mixture of 1,4-Dioxane (1.5 mL) and aqueous NaOH (4N, 1.5 mL) was heated at 150° C. under microwave irradiation for about 3 hrs. After being cooled to rt, the mixture was adjusted to pH 7 by AcOH and the solvent was removed in vacuo. The residual was purified by preparative HPLC to give 5-(8-chloroquinazolin-2-yl)pyridin-2-ol (Example 25, 15 mg, 21.2%) as a solid ¹H NMR (400 MHz, DMSO-d₆) δ ppm 11.96-12.09 (m, 1H), 9.66 (s, 1H), 8.56 (d, J=2.45 Hz, 1H), 8.51 (dd, J=2.69, 9.78 Hz, 1H), 8.17 (dd, J=1.22, 7.58 Hz, 1H), 8.12 (dd, J=1.22, 8.07 Hz, 1H), 7.66 (t, J=7.82 Hz, 1H), 6.55 (d, J=9.78 Hz, 1H). MS obsd. (ESI⁺) [(M+H)⁺]: 258.0

### Example 26

### 5-(8-Chloroquinazolin-2-yl)-N,N-dimethylpyridin-2-amine

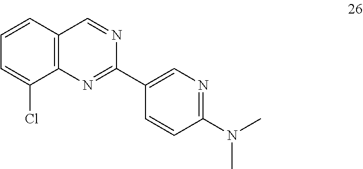 [SMILES: CN(C)c1ccc(-c2ncc3cccc(Cl)c3n2)cn1]

A mixture of 2-amino-3-chlorobenzaldehyde (100 mg, 0.643 mmol) and 6-chloronicotinimidamide hydrochloride (123 mg, 0.643 mmol) in DMA (2 mL) was heated at 210° C. under microwave irradiation for 1 hr. After being cooled to rt, the reaction mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by column chromatography on silica gel using gradient elution with PE-EtOAc from 100:20 to 100:100 (v/v) to give 5-(8-chloroquinazolin-2-yl)-N,N-dimethylpyridin-2-amine (Example 26, 57 mg, 28.3%) as a solid. ¹H NMR (400 MHz, DMSO-d₆) δ ppm 9.65 (s, 1H), 9.30 (d, J=1.71 Hz, 1H), 8.58 (dd, J=2.20, 9.05 Hz, 1H), 8.15 (dd, J=1.22, 7.58 Hz, 1H), 8.09 (dd, J=1.22, 8.31 Hz, 1H), 7.62 (t, J=7.83 Hz, 1H), 6.79-6.86 (m, 1H), 3.16 (s, 6H). MS obsd. (ESI⁺) [(M+H)⁺]: 285.1.

### Example 27

### 4-(5-(8-Chloroquinazolin-2-yl)pyridin-2-yl)morpholine

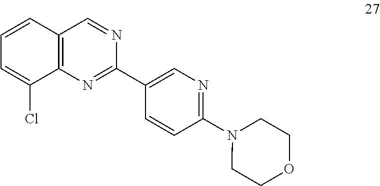 [SMILES: Clc1cccc2cnc(-c3ccc(N4CCOCC4)nc3)nc12]

A mixture of 8-chloro-2-(6-chloropyridin-3-yl)quinazoline (Example 24, 40 mg, 0.145 mmol) and morpholine (50.5 mg, 0.579 mmol) in 1,4-dioxane (3 mL) was heated at 140° C. under microwave irradiation for 2.5 hrs. After being cooled to rt, the solvent was removed in vacuo. The residue was purified by column chromatography on silica gel using gradient elution with PE-EtOAc from 100:20 to 100:100 (v/v) to give 4-(5-(8-chloroquinazolin-2-yl)pyridin-2-yl)morpholine (Example 27, 23 mg, 47.1%) as a solid. ¹H NMR (400 MHz, DMSO-d₆) δ ppm 9.68 (s, 1H), 9.32 (d, J=2.20 Hz, 1H), 8.62 (dd, J=2.45, 9.05 Hz, 1H), 8.17 (dd, J=1.22, 7.58 Hz, 1H), 8.11 (dd, J=1.22, 8.07 Hz, 1H), 7.65 (t, J=7.83 Hz, 1H), 7.03 (d, J=9.05 Hz, 1H), 3.70-3.77 (m, 4H), 3.60-3.67 (m, 4H). MS obsd. (ESI⁺) [(M+H)⁺]: 327.1.

### Example 28

### 2,2′-((5-(8-Chloroquinazolin-2-yl)pyridin-2-yl)azanediyl)diethanol

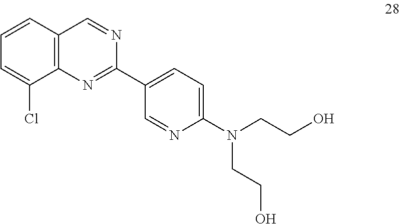 [SMILES: OCCN(CCO)c1ccc(-c2ncc3cccc(Cl)c3n2)cn1]

A mixture of 8-chloro-2-(6-chloropyridin-3-yl)quinazoline (Example 24, 50 mg, 0.181 mmol) and 2,2′-azanediyldiethanol (57.1 mg, 0.543 mmol) in NMP (2 mL) was stirred at 160° C. for 20 mins. After being cooled to rt, the reaction mixture was concentrated and the residue was purified by preparative HPLC to give 2,2′-((5-(8-chloroquinazolin-2-yl)pyridin-2-yl)azanediyl)diethanol (Example 28, 32 mg, 50.2%) as a solid. ¹H NMR (400 MHz, DMSO-d₆) δ ppm 9.65 (s, 1H), 9.26 (d, J=2.45 Hz, 1H), 8.54 (dd, J=2.32, 9.17 Hz, 1H), 8.15 (dd, J=1.22, 7.58 Hz, 1H), 8.08 (dd, J=1.22, 8.31 Hz, 1H), 7.62 (t, J=7.83 Hz, 1H), 6.86 (d, J=9.29 Hz, 1H), 4.83 (t, J=4.89 Hz, 2H), 3.66-3.72 (m, 4H), 3.60-3.66 (m, 4H). MS obsd. (ESI⁺) [(M+H)⁺]: 345.1.

### Example 29 and Example 30

### Methyl 1-(5-(8-chloroquinazolin-2-yl)pyridin-2-yl)piperidine-4-carboxylate (Example 29) and 1-(5-(8-chloroquinazolin-2-yl)pyridin-2-yl)piperidine-4-carboxylic acid (Example 30)

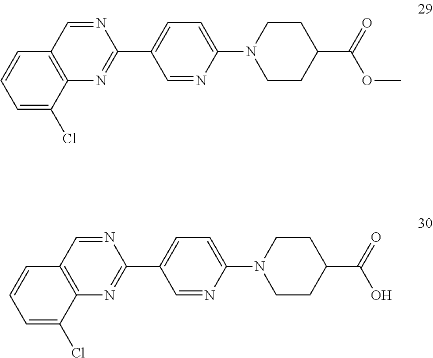 [SMILES: COC(=O)C1CCN(c2ccc(-c3ncc4cccc(Cl)c4n3)cn2)CC1 ; O=C(O)C1CCN(c2ccc(-c3ncc4cccc(Cl)c4n3)cn2)CC1]

### Preparation of methyl 1-(5-(8-chloroquinazolin-2-yl)pyridin-2-yl)piperidine-4-carboxylate (Example 29) and 1-(5-(8-chloroquinazolin-2-yl)pyridin-2-yl)piperidine-4-carboxylic acid (Example 30)

A mixture of 8-chloro-2-(6-chloropyridin-3-yl)quinazoline (compound 24, 50 mg, 0.181 mmol), methyl piperidine-4-carboxylate (38.9 mg, 0.272 mmol) and Et₃N (18.3 mg, 0.181 mmol) in NMP (3 mL) was heated at 170° C. under microwave irradiation for 1.5 hrs. After being cooled to rt, the reaction mixture concentrated and the residue was purified by preparative HPLC to give methyl 1-(5-(8-chloroquinazolin-2-yl)pyridin-2-yl)piperidine-4-carboxylate (Example 29, 15 mg, 21.2%) and 1-(5-(8-chloroquinazolin-2-yl)pyridin-2-yl)piperidine-4-carboxylic acid (Example 30, 21 mg, 30.8%) as solids.

Example 29: ¹H NMR (400 MHz, DMSO-d₆) δ ppm 9.69 (s, 1H), 9.22 (d, J=1.96 Hz, 1H), 8.64 (dd, J=2.20, 9.05 Hz, 1H), 8.17 (d, J=7.34 Hz, 1H), 8.12 (d, J=7.83 Hz, 1H), 7.66 (t, J=7.83 Hz, 1H), 7.15 (d, J=9.05 Hz, 1H), 4.35 (d, J=13.45 Hz, 2H), 3.64 (s, 3H), 3.18 (t, J=11.25 Hz, 2H), 2.68-2.82 (m, 1H), 1.97 (d, J=10.51 Hz, 2H), 1.54-1.70 (m, 2H). MS obsd. (ESI⁺) [(M+H)⁺]: 383.1.

Example 30: ¹H NMR (400 MHz, DMSO-d₆) δ ppm 9.68 (s, 1H), 9.23 (br. s., 1H), 8.62 (d, J=9.29 Hz, 1H), 8.17 (dd, J=0.98, 7.58 Hz, 1H), 8.11 (d, J=8.07 Hz, 1H), 7.65 (t, J=7.83 Hz, 1H), 7.13 (d, J=9.05 Hz, 1H), 4.35 (d, J=12.96 Hz, 2H), 3.16 (br. s., 2H), 2.60 (t, J=11.13 Hz, 1H), 1.94 (d, J=10.51 Hz, 2H), 1.52-1.67 (m, 2H). MS obsd. (ESI⁺) [(M+H)⁺]: 369.1.

### Example 31

### 2-(3-Bromo-phenyl)-7-trifluoromethyl-quinazoline-4-carboxylic acid

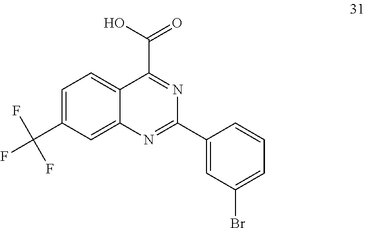 [SMILES: O=C(O)c1nc(-c2cccc(Br)c2)nc2cc(C(F)(F)F)ccc12]

### Step 1: Preparation of potassium 2-[2-amino-4-(trifluoromethyl)phenyl]-2-oxo-acetate

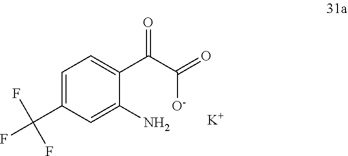 [SMILES: Nc1cc(C(F)(F)F)ccc1C(=O)C(=O)[O-] ; [K+]]

To a suspension solution of 6-(trifluoromethyl)indoline-2,3-dione (215 mg, 1.0 mmol) in water (1 mL) was added 1N KOH (1 mL, 1.0 mmol) at rt, then the mixture was stirred at 35-40° C. for 1 hr. After reaction was completed, the solvent was removed in vacuo to give a solid which was triturated with 5 mL of EtOH/diethyl ether (V/V=1/10) to give potassium 2-[2-amino-4-(trifluoromethyl)phenyl]-2-oxo-acetate (compound 31a, 160 mg, 59%) as a yellow solid. MS obsd. (ESI⁻) [(M−K)⁻]: 232.

### Step 2: Preparation of 2-(2-(3-bromobenzamido)-4-(trifluoromethyl)phenyl)-2-oxoacetic acid

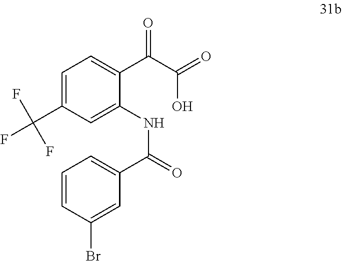 [SMILES: O=C(O)C(=O)c1ccc(C(F)(F)F)cc1NC(=O)c1cccc(Br)c1]

To a solution of potassium 2-[2-amino-4-(trifluoromethyl)phenyl]-2-oxo-acetate (compound 31a, 120 mg, 0.442 mmol) in THF (5 mL) were added triethylamine (134 mg, 1.33 mmol), 3-bromobenzoyl chloride (214 mg, 0.973 mmol) and DMAP (10.8 mg, 0.088 mmol) at 0° C., then the mixture was stirred at rt for 2 hrs. Water (1 mL) was added to the reaction mixture and stirred for additional 1 hr at rt, which was neutralized with 1N aqueous solution. The mixture was partitioned between EtOAc (10 mL) and water (10 mL), and the separated aqueous phase was extracted with EtOAc (10 mL) twice. The combined organic phase was dried over Na₂SO₄ and concentrated in vacuo to give the crude 2-(2-(3-bromobenzamido)-4-(trifluoromethyl)phenyl)-2-oxoacetic acid (compound 31b, 180 mg, 98%) as a semisolid, which was used in the next step directly without further purification. MS obsd. (ESI⁺) [(M+H)⁺]: 413.0.

### Step 3: Preparation of 2-(3-bromo-phenyl)-7-trifluoromethyl-quinazoline-4-carboxylic acid

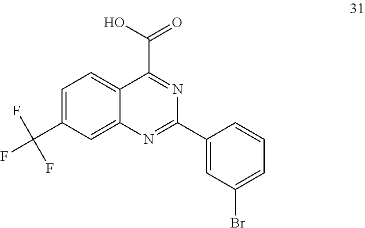 [SMILES: O=C(O)c1nc(-c2cccc(Br)c2)nc2cc(C(F)(F)F)ccc12]

A mixture of 2-(2-(3-bromobenzamido)-4-(trifluoromethyl)phenyl)-2-oxoacetic acid (compound 31b, 180 mg, 0.436 mmol) and ammonium acetate (295 mg, 3.83 mmol) in EtOH (3 mL) was heated at 90° C. under microwave irradiation for 30 mins. After being cooled to rt, the mixture was neutralized with 1N aqueous HCl solution. The mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by preparative HPLC to give 2-(3-bromo-phenyl)-7-trifluoromethyl-quinazoline-4-carboxylic acid (Example 31, 20 mg, 11.56%) as a solid. ¹H NMR (400 MHz, DMSO-d₆) δ ppm 8.71 (t, J=1.76 Hz, 1H), 8.58 (d, J=1.26 Hz, 1H), 8.56 (s, 1H), 8.48 (s, 1H), 8.02 (dd, J=1.76, 8.78 Hz, 1H), 7.81 (td, J=0.94, 7.91 Hz, 1H), 7.59 (t, J=7.91 Hz, 1H). MS obsd. (ESI⁺) [(M+H)⁺]: 397.0.

### Example 32

### Methyl 2-(3-bromophenyl)-7-(trifluoromethyl)quinazoline-4-carboxylate

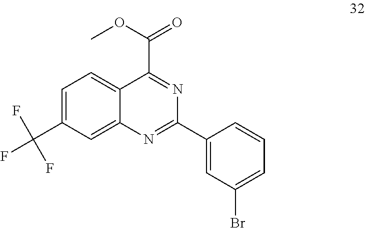 [SMILES: COC(=O)c1nc(-c2cccc(Br)c2)nc2cc(C(F)(F)F)ccc12]

### Preparation of methyl 2-(3-bromophenyl)-7-(trifluoromethyl)quinazoline-4-carboxylate

To a solution of 2-(3-bromo-phenyl)-7-trifluoromethyl-quinazoline-4-carboxylic acid (Example 31, 100 mg, 0.252 mol) in MeOH (3 mL) was added one-drop H₂SO₄ at rt, and the mixture was stirred at rt for 48 hrs. After the reaction was completed, the mixture was neutralized with NaHCO₃ and the solvent was removed in vacuo. The residue was partitioned between EtOAc (10 mL) and water (10 mL), and the separated aqueous phase was extracted with EtOAc (10 mL) twice. The combined organic phase was dried over Na₂SO₄ and concentrated in vacuo to give methyl 2-(3-bromophenyl)-7-(trifluoromethyl)quinazoline-4-carboxylate (Example 32, 100 mg, 91.8%) as a solid. ¹H NMR (400 MHz, DMSO-d₆) δ ppm 8.78 (d, J=8.78 Hz, 1H), 8.70 (t, J=1.76 Hz, 1H), 8.61 (s, 1H), 8.57 (d, J=8.03 Hz, 1H), 8.13 (dd, J=1.51, 8.78 Hz, 1H), 7.81-7.89 (m, 1H), 7.62 (t, J=7.91 Hz, 1H), 4.13 (s, 3H). MS obsd. (ESI⁺) [(M+H)⁺]: 411.2.

### Example 33 and Example 34

### 8-Chloro-2-(4-methoxyphenyl)quinazoline-4-carboxylic acid (Example 33) and 8-chloro-2-(4-methoxyphenyl)quinazoline-4-carboxamide (Example 34)

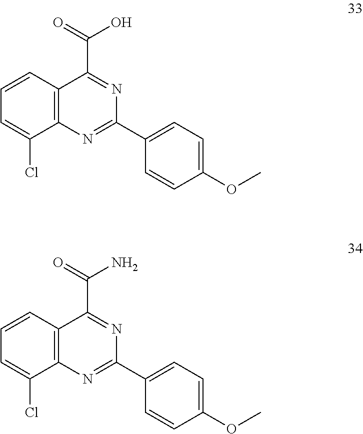 [SMILES: COc1ccc(-c2nc(C(=O)O)c3cccc(Cl)c3n2)cc1 ; COc1ccc(-c2nc(C(N)=O)c3cccc(Cl)c3n2)cc1]

### Step 1: Preparation of 7-chloro-1-(4-methoxybenzoyl)indoline-2,3-dione

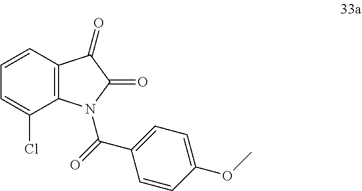 [SMILES: COc1ccc(C(=O)N2C(=O)C(=O)c3cccc(Cl)c32)cc1]

To a solution of 7-chloroindoline-2,3-dione (3 g, 16.5 mmol) in pyridine (16 mL) was added 4-methoxybenzoyl chloride (3.66 g, 21.5 mmol) under ice-water condition, then the mixture was heated at 60° C. for 1 hr. After being cooled to rt, the mixture was neutralized with 1N aqueous HCl. The mixture was partitioned between EtOAc (50 mL) and water (50 mL), and the separated aqueous phase was extracted with EtOAc (50 mL) twice. The combined organic phase was dried over Na₂SO₄ and concentrated in vacuo to give the crude 7-chloro-1-(4-methoxybenzoyl)indoline-2,3-dione (compound 33a, 3 g, 57.5%) as a brown solid, which was used in the next step directly without further purification. MS obsd. (ESI⁺) [(M+H)⁺]: 316.1.

### Step 2: Preparation of 8-chloro-2-(4-methoxyphenyl)quinazoline-4-carboxylic acid and 8-chloro-2-(4-methoxyphenyl)quinazoline-4-carboxamide

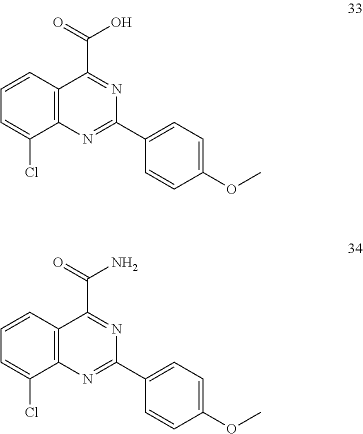 [SMILES: COc1ccc(-c2nc(C(=O)O)c3cccc(Cl)c3n2)cc1 ; COc1ccc(-c2nc(C(N)=O)c3cccc(Cl)c3n2)cc1]

A mixture of 7-chloro-1-(4-methoxybenzoyl)indoline-2,3-dione (compound 33a, 4 g, 12.7 mmol) and ammonium acetate (9.77 g, 127 mmol) in EtOH (100 mL) was heated at 90° C. under microwave irradiation for 30 mins. After being cooled to rt, the mixture was neutralized with 1N aqueous HCl solution. The mixture was partitioned between EtOAc (100 mL) and water (100 mL), and the separated aqueous phase was extracted with EtOAc (100 mL) twice. The combined organic phase was dried over Na₂SO₄ and concentrated in vacuo. The residue was purified by preparative HPLC to give 8-chloro-2-(4-methoxyphenyl)quinazoline-4-carboxylic acid (Example 33, 0.2 g, 5.02%) and 8-chloro-2-(4-methoxyphenyl)quinazoline-4-carboxamide (Example 34, 0.6 g, 15.1%) as yellow solids.

Example 33: ¹H NMR (400 MHz, METHANOL-d₄) δ ppm 8.68 (d, J=9.05 Hz, 2H), 8.43-8.50 (m, 1H), 8.07-8.15 (m, 1H), 7.58-7.67 (m, 1H), 7.11 (d, J=8.93 Hz, 1H), 7.05-7.15 (m, 1H), 3.93 (s, 3H). MS obsd. (ESI⁺) [(M+H)⁺]: 315.0.

Example 34: ¹H NMR (400 MHz, DMSO-d₆) δ ppm 8.71 (dd, J=8.44, 1.22 Hz, 1H), 8.61-8.67 (m, 3H), 8.13-8.26 (m, 2H), 7.68 (dd, J=8.38, 7.64 Hz, 1H), 7.09-7.22 (m, 2H), 3.32 (s, 3H). MS obsd. (ESI⁺) [(M+H)⁺]: 314.0.

### Example 35

### 8-Chloro-N-(2-hydroxyethyl)-2-(4-methoxyphenyl)quinazoline-4-carboxamide

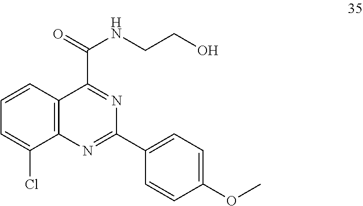 [SMILES: COc1ccc(-c2nc(C(=O)NCCO)c3cccc(Cl)c3n2)cc1]

### Preparation of 8-chloro-N-(2-hydroxyethyl)-2-(4-methoxyphenyl)quinazoline-4-carboxamide

To a solution of 8-chloro-2-(4-methoxyphenyl)quinazoline-4-carboxylic acid (Example 33, 250 mg, 0.794 mmol) in DCM (20 mL) was added oxalyl dichloride (106 mg, 0.834 mmol) dropwise and 2 drops of DMF at 0° C., and the reaction mixture was stirred at rt for 2 hrs. Then the reaction mixture was added to a solution of 2-aminoethanol (229 mg, 3.75 mmol) and triethylamine (152 mg, 1.5 mmol) in DCM (10 mL) at rt, which was stirred at rt for additional 30 mins. After quenched with water, the reaction mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by preparative HPLC to give 8-chloro-N-(2-hydroxyethyl)-2-(4-methoxyphenyl)quinazoline-4-carboxamide (Example 35, 250 mg, 91.3%) as a yellow solid. ¹H NMR (400 MHz, DMSO-d₆) δ ppm 9.19 (br t, J=5.69 Hz, 1H), 8.75 (dd, J=8.50, 1.16 Hz, 1H), 8.60-8.68 (m, 2H), 8.22 (dd, J=7.58, 1.22 Hz, 1H), 7.68 (dd, J=8.31, 7.70 Hz, 1H), 7.11-7.20 (m, 2H), 4.87 (t, J=5.50 Hz, 1H), 3.89 (s, 3H), 3.63 (q, J=5.87 Hz, 2H), 3.50 (q, J=6.19 Hz, 2H). MS obsd. (ESI⁺) [(M+H)⁺]: 358.2.

### Example 36

### 8-Chloro-2-(4-methoxyphenyl)quinazoline

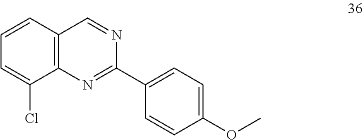 [SMILES: COc1ccc(-c2ncc3cccc(Cl)c3n2)cc1]

A mixture of 8-chloro-2-(4-methoxyphenyl)quinazoline-4-carboxylic acid (Example 33, 30 mg, 0.095 mmol) in NMP (2 ml) was charged with Aragon, and the mixture was heated at 140° C. under microwave irradiation for 1 hr. Then the mixture was concentrated in vacuo. The residue was purified by preparative HPLC to give 8-chloro-2-(4-methoxyphenyl)quinazoline (Example 36, 20 mg, 76%) as a white solid. ¹H NMR (400 MHz, METHANOL-d₄) δ ppm 9.42 (s, 1H), 8.46-8.54 (m, 2H), 7.96 (dd, J=7.52, 1.28 Hz, 1H), 7.90 (dd, J=8.07, 1.22 Hz, 1H), 7.49 (t, J=7.82 Hz, 1H), 6.90-7.03 (m, 2H), 3.80 (s, 3H). MS obsd. (ESI⁺) [(M+H)⁺]: 271.2.

### Example 37 and Example 38

### 2-(4-Bromophenyl)-8-chloroquinazoline-4-carboxylic acid (Example 37) and ethyl 2-(4-bromophenyl)-8-chloro-quinazoline-4-carboxylate (Example 38)

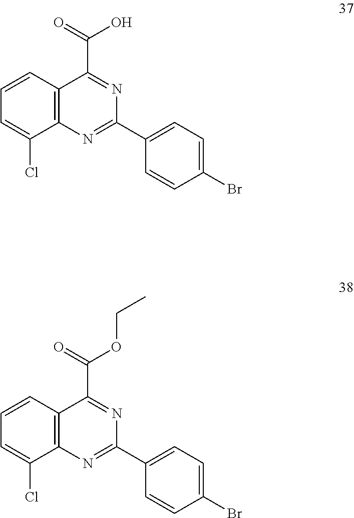 [SMILES: O=C(O)c1nc(-c2ccc(Br)cc2)nc2c(Cl)cccc12 ; CCOC(=O)c1nc(-c2ccc(Br)cc2)nc2c(Cl)cccc12]

### Step 1: Preparation of 1-(4-bromobenzoyl)-7-chloroindoline-2,3-dione

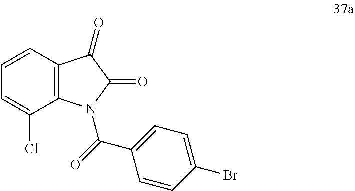 [SMILES: O=C1C(=O)N(C(=O)c2ccc(Br)cc2)c2c(Cl)cccc21]

Compound 37a was prepared in analogy to Example 33, step 1 by using 4-bromobenzoyl chloride (2.66 g, 12.1 mmol) instead of 4-methoxybenzoyl chloride in to give 1-(4-bromobenzoyl)-7-chloroindoline-2,3-dione (compound 37a, 4 g, 99.6%) as a brown solid, which was used in the next step directly without further purification. MS obsd. (ESI⁺) [(M+H)⁺]: MS obsd. (ESI⁺) [(M+H)⁺]: 364.0&366.0.

### Step 2: Preparation of 2-(4-bromophenyl)-8-chloroquinazoline-4-carboxylic acid (Example 37) and ethyl 2-(4-bromophenyl)-8-chloro-quinazoline-4-carboxylate (Example 38)

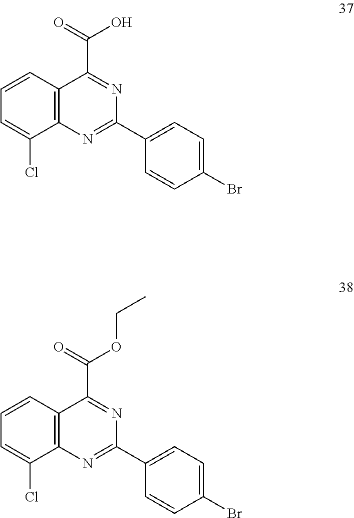 [SMILES: O=C(O)c1nc(-c2ccc(Br)cc2)nc2c(Cl)cccc12 ; CCOC(=O)c1nc(-c2ccc(Br)cc2)nc2c(Cl)cccc12]

Example 37 and Example 38 were prepared in analogy to Example 33, step 2 by using 1-(4-bromobenzoyl)-7-chloroindoline-2,3-dione (compound 37a, 5 g, 13.7 mmol) instead of 7-chloro-1-(4-methoxybenzoyl)indoline-2,3-dione (compound 33a). 2-(4-Bromophenyl)-8-chloroquinazoline-4-carboxylic acid (Example 37, 4 g) and ethyl 2-(4-bromophenyl)-8-chloro-quinazoline-4-carboxylate (Example 38, 1.8 g, 41.9%) were obtained as white solids.

Example 37: ¹H NMR (400 MHz, DMSO-d₆) δ ppm 8.52 (d, J=8.31 Hz, 2H), 8.25 (dd, J=8.07, 12.96 Hz, 2H), 7.82 (d, J=8.56 Hz, 2H), 7.71 (t, J=7.95 Hz, 1H). MS obsd. (ESI⁺) [(M+H)⁺]: 362.9. MS obsd. (ESI⁺) [(M+H)⁺]: 363.2&365.2.

Example 38: ¹H NMR (400 MHz, DMSO-d₆) δ ppm 8.50 (d, J=8.68 Hz, 2H), 8.30-8.42 (m, 2H), 7.79-7.89 (m, 3H), 4.59 (q, J=7.17 Hz, 2H), 1.44 (t, J=7.09 Hz, 3H). MS obsd. (ESI⁺) [(M+H)⁺]: 391.0&393.0.

### Example 39

### 2-(4-Bromophenyl)-8-chloro-N-(2-chloroethyl)quinazoline-4-carboxamide

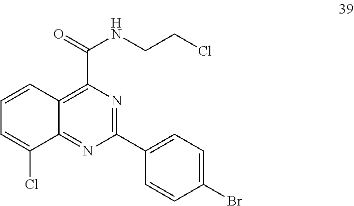 [SMILES: O=C(NCCCl)c1nc(-c2ccc(Br)cc2)nc2c(Cl)cccc12]

### Preparation of 2-(4-bromophenyl)-8-chloro-N-(2-chloroethyl)quinazoline-4-carboxamide

Example 39 was prepared in analogy to Example 35 by using 2-(4-bromophenyl)-8-chloroquinazoline-4-carboxylic acid (Example 37, 200 mg, 0.55 mmol) and 2-chloroethanamine hydrochloride (182 mg, 1.57 mmol) instead of 8-chloro-2-(4-methoxyphenyl)quinazoline-4-carboxylic acid (Example 33) and 2-aminoethanol. 2-(4-Bromophenyl)-8-chloro-N-(2-chloroethyl)quinazoline-4-carboxamide (Example 39, 160 mg) was obtained as an off-white solid. ¹H NMR (400 MHz, DMSO-d₆) δ ppm 9.46-9.56 (m, 1H), 8.78-8.85 (m, 1H), 8.65 (d, J=8.68 Hz, 2H), 8.28 (s, 1H), 7.82-7.92 (m, 2H), 7.73-7.81 (m, 1H), 3.82-3.92 (m, 2H), 3.73-3.80 (m, 2H). MS obsd. (ESI⁺) [(M+H)⁺]: 426.0.

### Example 40

### 2-(4-Bromophenyl)-8-chloro-4-methyl-quinazoline

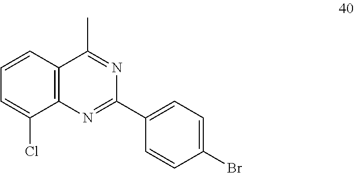 [SMILES: Cc1nc(-c2ccc(Br)cc2)nc2c(Cl)cccc12]

### Preparation of 2-(4-bromophenyl)-8-chloro-4-methyl-quinazoline

To a solution of 2-(4-bromophenyl)-8-chloroquinazoline-4-carboxylic acid (Example 37, 300 mg, 825 μmol) in tetrahydrofuran (10 mL) was added methylmagnesium bromide (4.13 ml, 4.13 mmol) dropwise at 0° C. After warmed to rt and stirred for 2 hrs, the mixture was quenched with saturated aqueous NH₄Cl (10 mL) and extracted with EtOAc (50 mL) and washed with saturated aqueous NH₄Cl (50 mL) three times. The organic layer was separated, dried over Na₂SO₄ and concentrated in vacuo. The residue was purified by preparative HPLC to give 2-(4-bromophenyl)-8-chloro-4-methyl-quinazoline (Example 40, 22 mg, 7.83%) as a white solid. ¹H NMR (400 MHz, DMSO-d₆) δ ppm 8.50-8.58 (m, 2H), 8.30 (dd, J=8.31, 1.22 Hz, 1H), 8.19 (dd, J=7.58, 1.10 Hz, 1H), 7.76-7.87 (m, 2H), 7.65-7.75 (m, 1H), 3.04 (s, 3H). MS obsd. (ESI⁺) [(M+H)⁺]: 333.0&335.0.

### Example 41

### 2-[4-(8-Chloroquinazolin-2-yl)phenoxy]ethanol

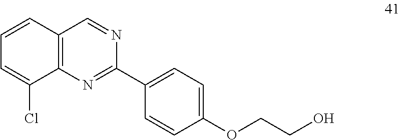 [SMILES: OCCOc1ccc(-c2ncc3cccc(Cl)c3n2)cc1]

### Step 1: Preparation of 8-chloro-2-(4-(2-hydroxyethoxy)phenyl)quinazoline-4-carboxylic acid

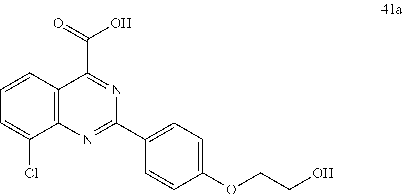 [SMILES: O=C(O)c1nc(-c2ccc(OCCO)cc2)nc2c(Cl)cccc12]

A mixture of 2-(4-bromophenyl)-8-chloroquinazoline-4-carboxylic acid (Example 37, 500 mg, 1.38 mmol), potassium carbonate (570 mg, 4.13 mmol) and cupric chloride (92.4 mg, 0.688 mmol) in ethane-1,2-diol (1.71 g, 27.5 mmol) was stirred at 130° C. for 36 hrs. The mixture was diluted with EtOAc (100 mL) and washed with saturated aqueous NH₄Cl (50 ml 3 times). The organic layer was separated, dried over Na₂SO₄ and concentrated in vacuo. The residue was purified by column chromatography on silica gel using gradient elution with PE-EtOAc from 100:10 to 100:100 (v/v) to give 8-chloro-2-(4-(2-hydroxyethoxy)phenyl)quinazoline-4-carboxylic acid (compound 41a, 400 mg, 84.4%) as a white solid. MS obsd. (ESI⁺) [(M+H)⁺]: 345.2.

### Step 2: Preparation of 2-[4-(8-chloroquinazolin-2-yl)phenoxy]ethanol

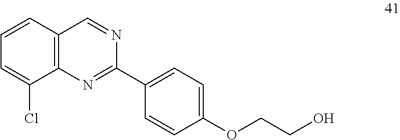 [SMILES: OCCOc1ccc(-c2ncc3cccc(Cl)c3n2)cc1]

A mixture of 8-chloro-2-(4-(2-hydroxyethoxy)phenyl)quinazoline-4-carboxylic acid (compound 41a, 400 mg, 1.16 mmol) in ethylene glycol (14.4 g, 232 mmol) was charged with Argon, and the mixture was heated at 150° C. under microwave irradiation for 3 hrs. Then the mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by preparative HPLC to give 2-[4-(8-chloroquinazolin-2-yl)phenoxy]ethanol (Example 41, 300 mg, 84.3%) as a light yellow solid. ¹H NMR (400 MHz, METHANOL-d₄) δ ppm 9.55 (s, 1H), 8.64 (d, J=8.93 Hz, 2H), 7.98-8.11 (m, 2H), 7.62 (d, J=7.95 Hz, 1H), 7.13 (d, J=8.93 Hz, 2H), 4.18 (d, J=4.89 Hz, 2H), 3.95 (s, 2H). MS obsd. (ESI⁺) [(M+H)⁺]: 301.1.

### Example 42

### cis-3-[2-[4-(8-Chloroquinazolin-2-yl)phenoxy]ethoxy]cyclobutanecarboxylic acid

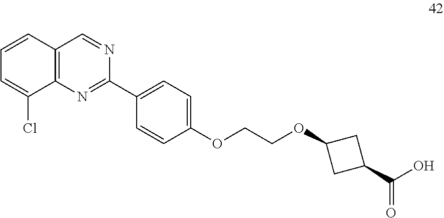 [SMILES: O=C(O)[C@H]1C[C@@H](OCCOc2ccc(-c3ncc4cccc(Cl)c4n3)cc2)C1]

### Step 1: Preparation of cis-methyl 3-[2-[4-(8-chloroquinazolin-2-yl)phenoxy]ethoxy]cyclobutanecarboxylate

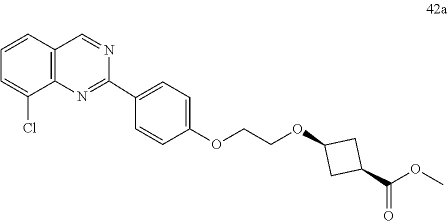 [SMILES: COC(=O)[C@H]1C[C@@H](OCCOc2ccc(-c3ncc4cccc(Cl)c4n3)cc2)C1]

To a solution of 2-[4-(8-chloroquinazolin-2-yl)phenoxy]ethanol (Example 41.90 mg, 0.299 mmol) and 2,6-lutidine (48.1 mg, 0.449 mmol) in DCM (5 mL) was added trifluoromethanesulfonic anhydride (127 mg, 0.449 mmol) dropwise at 0° C., and the mixture was stirred for 30 min. The mixture diluted with EtOAc (20 mL) and was washed with water (10 mL) and 0.5 N aqueous HCl (10 mL) and then water (10 mL). The organic layer was separated and dried over anhydrous Na₂SO₄. After filtered off the desiccant, the filtrated was concentrated, and the residue was added to a previous prepared mixture of cis-methyl 3-hydroxycyclobutanecarboxylate (77.9 mg, 0.599 mmol) and sodium hydride (7.18 mg, 0.299 mmol) in THF (5 mL) at 0° C. The mixture was stirred for 1 hr, then quenched by water. The mixture was extracted with EtOAc (30 mL) and washed with water (10 mL). The organic layer was separated and concentrated in vacuo. The residue was purified by column chromatography on silica gel using gradient elution with PE-EtOAc from 100:10 to 100:50 (v/v) to give cis-methyl 3-(2-(4-(8-chloroquinazolin-2-yl)phenoxy)ethoxy)cyclobutanecarboxylate (compound 42a, 70 mg, 56.7%) as a light yellow solid. MS obsd. (ESI⁺) [(M+H)⁺]: 413.2.

### Step 2: Preparation of cis-3-[2-[4-(8-chloroquinazolin-2yl)phenoxy]ethoxy]cyclobutanecarboxylic acid

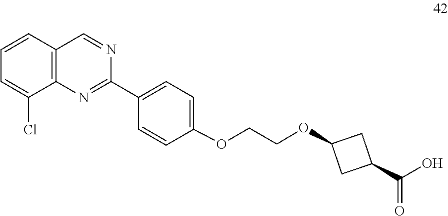 [SMILES: O=C(O)[C@H]1C[C@@H](OCCOc2ccc(-c3ncc4cccc(Cl)c4n3)cc2)C1]

To a solution of cis-methyl 3-(2-(4-(8-chloroquinazolin-2-yl)phenoxy)ethoxy)cyclobutanecarboxylate (compound 42a, 65 mg, 0.157 mmol) in THF (5 mL) and water (5 mL) was added lithium hydroxide monohydrate (19.8 mg, 0.472 mmol), and the mixture was stirred for 2 hrs. After the solvent was removed in vacuo, the remained aqueous solution was acidified by 1N aqueous HCl to pH 2 and extracted with EtOAc (30 mL). The organic layer was separated and dried over anhydrous Na₂SO₄ and concentrated in vacuo to give cis-3-[2-[4-(8-chloroquinazolin-2-yl)phenoxy]ethoxy]cyclobutanecarboxylic acid (Example 42, 36 mg, 56.8%) as a white solid. ¹H NMR (DMSO-d₆, 400 MHz)): δ ppm 12.16 (br s, 1H), 9.72 (s, 1H), 8.56 (d, J=8.93 Hz, 2H), 8.19 (dd, J=7.52, 1.28 Hz, 1H), 8.13 (dd, J=8.13, 1.28 Hz, 1H), 7.67 (t, J=7.83 Hz, 1H), 7.16 (d, J=9.05 Hz, 2H), 4.15-4.22 (m, 2H), 3.91-4.01 (m, 1H), 3.64-3.72 (m, 2H), 2.54-2.65 (m, 1H), 2.41-2.48 (m, 2H), 1.95-2.06 (m, 2H). MS obsd. (ESI⁺) [(M+H)⁺]: 399.2.

### Example 43

### 2-(4-Bromophenyl)-7-(trifluoromethyl)quinazoline-4-carboxylic acid

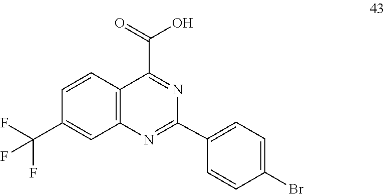 [SMILES: O=C(O)c1nc(-c2ccc(Br)cc2)nc2cc(C(F)(F)F)ccc12]

### Step 1: Preparation of 1-(4-bromobenzoyl)-6-(trifluoromethyl)indoline-2,3-dione

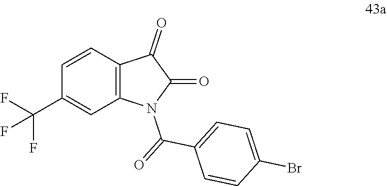 [SMILES: O=C1C(=O)N(C(=O)c2ccc(Br)cc2)c2cc(C(F)(F)F)ccc21]

1-(4-Bromobenzoyl)-6-(trifluoromethyl)indoline-2,3-dione was prepared in analogy to Example 33, step 1 by using 4-bromobenzoyl chloride (2.66 g, 12.1 mmol) and 6-(trifluoromethyl)indoline-2,3-dione instead of 4-methoxybenzoyl chloride and 7-chloroindoline-2,3-dione. 1-(4-bromobenzoyl)-6-(trifluoromethyl)indoline-2,3-dione was obtained as a brown solid (compound 43a, 0.9 g, 97.3%), which was used in the next step directly without further purification. MS obsd. (ESI⁺) [(M+H)⁺]: 398.1, 400.1.

### Step 2: Preparation of 2-(4-bromophenyl)-7-(trifluoromethyl)quinazoline-4-carboxylic acid

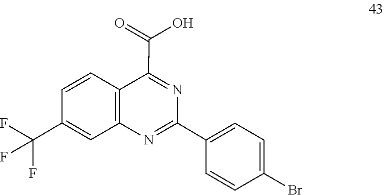 [SMILES: O=C(O)c1nc(-c2ccc(Br)cc2)nc2cc(C(F)(F)F)ccc12]

Example 43 was prepared in analogy to Example 33, step 2 by using 1-(4-bromobenzoyl)-6-(trifluoromethyl)indoline-2,3-dione (compound 43a) instead of 7-chloro-1-(4-methoxybenzoyl)indoline-2,3-dione (compound 33a). 2-(4-Bromophenyl)-7-(trifluoromethyl)quinazoline-4-carboxylic acid was obtained as a white solid (Example 43, 898 mg, 89.1%) after purified by column chromatography on silica gel using gradient elution with PE-EtOAc from 100:10 to 100:100 (v/v). ¹H NMR (400 MHz, DMSO-d₆) δ ppm 8.72 (d, J=8.80 Hz, 1H), 8.48-8.58 (m, 3H), 8.08 (dd, J=1.71, 8.80 Hz, 1H), 7.78-7.88 (m, 2H). MS obsd. (ESI⁺) [(M+H)⁺]: 397.2&399.2.

### Example 44

### [2-(4-Bromophenyl)-7-(trifluoromethyl)quinazolin-4-yl]-morpholino-methanone

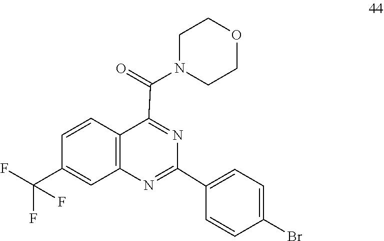 [SMILES: O=C(c1nc(-c2ccc(Br)cc2)nc2cc(C(F)(F)F)ccc12)N1CCOCC1]

### Preparation of [2-(4-bromophenyl)-7-(trifluoromethyl)quinazolin-4-yl]-morpholino-methanone

To a mixture of 2-(4-bromophenyl)-7-(trifluoromethyl)quinazoline-4-carboxylic acid (Example 43, 100 mg, 252 μmol), morpholine (43.9 mg, 44 μL, 504 μmol) and triethylamine (76.4 mg, 0.755 mmol) in DCM (2 mL) and DMF (2 mL) was added HATU (144 mg, 0.378 mmol) and the mixture was stirred for 20 min at rt. After the reaction was completed, the reaction mixture was diluted with water (10 mL) and extracted with EtOAc (20 mL). The combined organic phase was dried over Na₂SO₄ and concentrated in vacuo. The residue was purified by preparative HPLC to give [2-(4-bromophenyl)-7-(trifluoromethyl)quinazolin-4-yl]-morpholino-methanone (Example 44, 9 mg, 7.51%) as a white solid. ¹H NMR (400 MHz, METHANOL-d₄) δ ppm 8.54-8.62 (m, 2H), 8.50 (s, 1H), 8.29 (d, J=8.68 Hz, 1H), 7.98 (dd, J=8.68, 1.59 Hz, 1H), 7.72-7.80 (m, 2H), 3.99 (s, 2H), 3.88-3.94 (m, 2H), 3.64-3.71 (m, 2H), 3.39-3.46 (m, 2H). MS obsd. (ESI⁺) [(M+H)⁺]: 466.2&468.2.

### Example 45 and Example 46

### 8-Chloro-2-(6-chloro-3-pyridyl)quinazoline-4-carboxylic acid (Example 45) and 8-chloro-2-(6-chloro-3-pyridyl)quinazoline-4-carboxamide (Example 46)

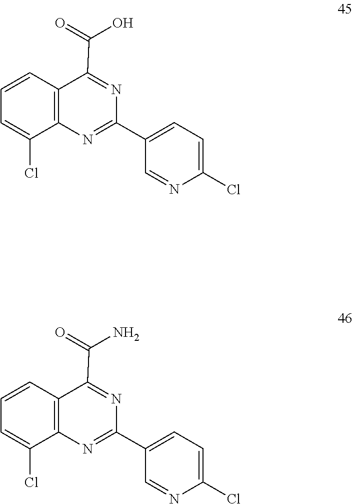 [SMILES: O=C(O)c1nc(-c2ccc(Cl)nc2)nc2c(Cl)cccc12 ; NC(=O)c1nc(-c2ccc(Cl)nc2)nc2c(Cl)cccc12]

### Step 1: Preparation of 7-chloro-1-(6-chloronicotinoyl)indoline-2,3-dione

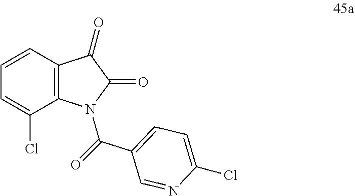 [SMILES: O=C1C(=O)N(C(=O)c2ccc(Cl)nc2)c2c(Cl)cccc21]

Compound 45a was prepared in analogy to Example 33, step 1 by using 6-chloronicotinoyl chloride (1.07 g, 6.06 mmol) instead of 4-methoxybenzoyl chloride. 7-Chloro-1-(6-chloronicotinoyl)indoline-2,3-dione (compound 45a, 1.5 g) was obtained as a brown solid, which was used in the next step directly without further purification. MS obsd. (ESI⁺) [(M+H)⁺]: 321.0

### Step 2: Preparation of ethyl 8-chloro-2-(6-chloropyridin-3-yl)quinazoline-4-carboxylate (compound 45b), 8-chloro-2-(6-chloro-3-pyridyl)quinazoline-4-carboxylic acid (Example 45) and 8-chloro-2-(6-chloro-3-pyridyl)quinazoline-4-carboxamide (Example 46)

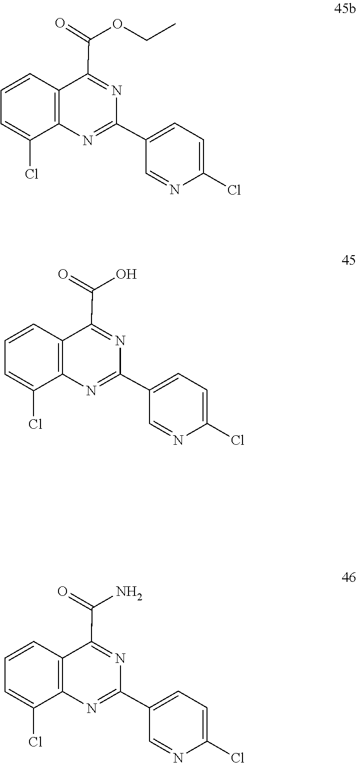 [SMILES: CCOC(=O)c1nc(-c2ccc(Cl)nc2)nc2c(Cl)cccc12 ; O=C(O)c1nc(-c2ccc(Cl)nc2)nc2c(Cl)cccc12 ; NC(=O)c1nc(-c2ccc(Cl)nc2)nc2c(Cl)cccc12]

Compound 45b, Example 45 and Example 46 were prepared in analogy to Example 33, step 2 by using 7-chloro-1-(6-chloronicotinoyl)indoline-2,3-dione (compound 45a, 1.6 g, 4.98 mmol) as the starting materials instead of 7-chloro-1-(4-methoxybenzoyl)indoline-2,3-dione (compound 33a). After prep-HPLC purification and separation, ethyl 8-chloro-2-(6-chloropyridin-3-yl)quinazoline-4-carboxylate (compound 45b, 0.28 g, 16.1%), 8-Chloro-2-(6-chloro-3-pyridyl)quinazoline-4-carboxylic acid (Example 45, 500 mg, 31.3%), 8-Chloro-2-(6-chloro-3-pyridyl)quinazoline-4-carboxamide (Example 46, 200 mg, 12.6%) were obtained.

Compound 45b: MS obsd. (ESI⁺) [(M+H)⁺]: 348.0.

Example 45: ¹H NMR (400 MHz, METHANOL-d₄) δ ppm 9.50 (s, 1H), 7.52-8.92 (br d, J=7.95 Hz, 1H), 8.27 (br s, 1H), 8.03 (d, J=7.58 Hz, 1H), 7.62 (m, 2H). MS obsd. (ESI⁺) [(M+H)⁺]: 320.0.

Example 46: ¹H NMR (400 MHz, DMSO-d₆) δ ppm 9.62-9.69 (m, 1H), 8.97-9.06 (m, 1H), 8.88-8.97 (m, 1H), 8.75-8.88 (m, 1H), 8.28-8.37 (m, 1H), 8.20-8.28 (m, 1H), 7.80 (br d, J=8.44 Hz, 2H). MS obsd. (ESI⁺) [(M+H)⁺]: 319.0.

### Example 47

### 8-Chloro-2-(6-methoxy-3-pyridyl)quinazoline-4-carboxylic acid

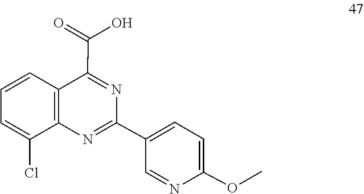 [SMILES: COc1ccc(-c2nc(C(=O)O)c3cccc(Cl)c3n2)cn1]

### Preparation of 8-chloro-2-(6-methoxy-3-pyridyl)quinazoline-4-carboxylic acid

A mixture of ethyl 8-chloro-2-(6-chloropyridin-3-yl)quinazoline-4-carboxylate (compound 45b, 0.25 g, 718 μmol) and sodium methoxide (194 mg, 3.59 mmol) in MeOH (20 mL) was stirred at 75° C. for 16 hrs. The mixture was neutralized with 1N aqueous HCl solution, and partitioned between EtOAc (10 mL) and water (10 mL), then the separated aqueous phase was extracted with EtOAc (10 mL) twice. The combined organic phase was dried over Na₂SO₄ and concentrated in vacuo. The residue was purified by preparative HPLC to give 8-chloro-2-(6-methoxy-3-pyridyl)quinazoline-4-carboxylic acid (Example 47, 180 mg, 75.4%) as a yellow solid. ¹H NMR (400 MHz, DMSO-d₆) δ ppm 9.34 (d, J=1.96 Hz, 1H), 8.77 (dd, J=8.74, 2.38 Hz, 1H), 8.38 (dd, J=8.38, 1.16 Hz, 1H), 8.28 (dd, J=7.58, 1.10 Hz, 1H), 7.70-7.78 (m, 1H), 7.06 (d, J=8.80 Hz, 1H), 3.98 (s, 3H). MS obsd. (ESI⁺) [(M+H)⁺]: 316.1.

### Example 48

### 8-Chloro-2-(6-ethoxy-3-pyridyl)quinazoline-4-carboxylic acid

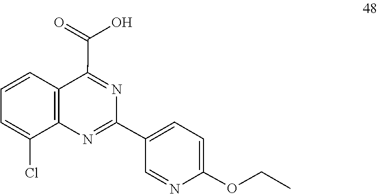 [SMILES: CCOc1ccc(-c2nc(C(=O)O)c3cccc(Cl)c3n2)cn1]

### Preparation of 8-chloro-2-(6-ethoxy-3-pyridyl)quinazoline-4-carboxylic acid

Example 48 was prepared in analogy to Example 47 by using sodium ethanolate (266 mg, 3.9 mmol) in EtOH (20 mL) instead of sodium methoxide in MeOH. 8-Chloro-2-(6-ethoxy-3-pyridyl)quinazoline-4-carboxylic acid (Example 48, 150 mg) was obtained as brown solid. ¹H NMR (400 MHz, DMSO-d₆) δ ppm 9.32 (d, J=2.45 Hz, 1H), 8.75 (dd, J=8.68, 2.45 Hz, 1H), 8.22-8.41 (m, 2H), 7.75 (dd, J=8.31, 7.70 Hz, 1H), 6.96-7.09 (m, 1H), 4.43 (d, J=7.09 Hz, 2H), 1.38 (t, J=7.09 Hz, 3H). MS obsd. (ESI⁺) [(M+H)⁺]: 330.1.

### Example 49

### 8-Chloro-2-(6-pyrrolidin-1-yl-3-pyridyl)quinazoline-4-carboxylic acid

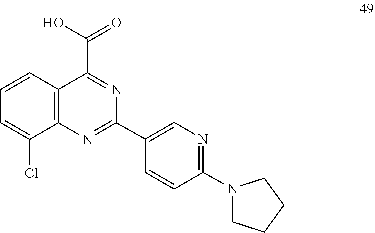 [SMILES: O=C(O)c1nc(-c2ccc(N3CCCC3)nc2)nc2c(Cl)cccc12]

A mixture of ethyl 8-chloro-2-(6-chloropyridin-3-yl)quinazoline-4-carboxylate (compound 45b, 100 mg, 287 μmol), pyrrolidine (30.6 mg, 0.431 mmol) and potassium phosphate tribasic (122 mg, 0.574 mmol) in DMF (10 mL) was stirred at 90° C. for 3 hrs. The mixture was neutralized with 1N aqueous HCl solution. The mixture was partitioned between EtOAc (10 mL) and water (10 mL), and the separated aqueous phase was extracted with EtOAc (10 mL) twice. The combined organic phase was dried over Na₂SO₄ and concentrated in vacuo. The residue was purified by preparative HPLC to give 8-chloro-2-(6-pyrrolidin-1-yl-3-pyridyl)quinazoline-4-carboxylic acid (Example 49, 50 mg, 49.1%) as a white solid. ¹H NMR (400 MHz, DMSO-d₆) δ ppm 9.01 (s, 1H), 8.78 (br d, J=9.41 Hz, 1H), 8.38 (dd, J=8.38, 1.04 Hz, 1H), 8.29 (dd, J=7.58, 1.10 Hz, 1H), 7.76 (t, J=8.01 Hz, 1H), 7.12-7.23 (m, 1H), 3.65 (m, 4H), 2.00-2.12 (m, 4H). MS obsd. (ESI⁺) [(M+H)⁺]: 355.0.

### Example 50

### cis-2-[6-[2-(3-Carboxycyclobutoxy)ethoxy]-3-pyridyl]-8-chloro-quinazoline-4-carboxylic acid

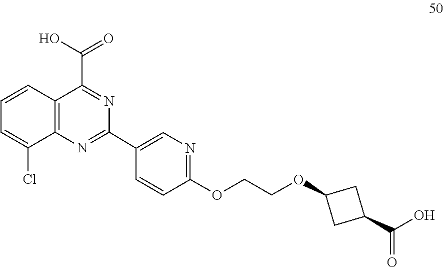 [SMILES: O=C(O)c1nc(-c2ccc(OCCO[C@H]3C[C@@H](C(=O)O)C3)nc2)nc2c(Cl)cccc12]

To a stirred mixture solution of cis-methyl 3-(2-hydroxyethoxy)cyclobutanecarboxylate (218 mg, 1.25 mmol) in THF (20 mL) was added sodium hydride (250 mg, 6.25 mmol) and stirred at rt for 0.5 hr. Then a solution of 8-chloro-2-(6-chloro-3-pyridyl)quinazoline-4-carboxylic acid (Example 45, 200 mg, 0.625 mmol) in DMF (10 mL) was added to the above reaction mixture. The reaction mixture was heated to 60° C. for 7 hrs, then neutralized with 1N aqueous HCl solution and partitioned between EtOAc (10 mL) and water (10 mL). The separated aqueous phase was extracted with EtOAc (10 mL) twice. The combined organic phase was dried over Na₂SO₄ and concentrated in vacuo. The residue was purified by preparative HPLC to give cis-2-[6-[2-(3-carboxycyclobutoxy)ethoxy]-3-pyridyl]-8-chloro-quinazoline-4-carboxylic acid (Example 50, 93 mg, 32.9%) as a white solid. ¹H NMR (400 MHz, DMSO-d₆) δ ppm 11.98-12.34 (m, 1H), 9.28-9.35 (m, 1H), 8.78 (dd, J=8.80, 2.45 Hz, 1H), 8.38 (dd, J=8.44, 1.22 Hz, 1H), 8.29 (dd, J=7.58, 1.22 Hz, 1H), 7.76 (dd, J=8.44, 7.70 Hz, 1H), 7.09 (d, J=9.29 Hz, 1H), 4.42-4.53 (m, 2H), 3.95 (quin, J=7.37 Hz, 1H), 3.61-3.75 (m, 2H), 2.55-2.63 (m, 1H), 2.44 (br d, J=6.85 Hz, 2H), 1.99 (br d, J=10.88 Hz, 2H). MS obsd. (ESI⁺) [(M+H)⁺]: 444.0.

### Example 51

### 2-[2-[[5-(8-Chloroquinazolin-2-yl)-2-pyridyl]oxy]ethoxy]acetic acid

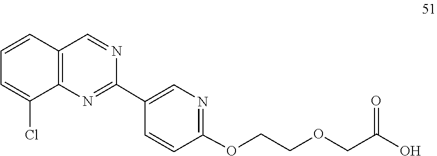 [SMILES: O=C(O)COCCOc1ccc(-c2ncc3cccc(Cl)c3n2)cn1]

### Step 1: Preparation of 2-(6-(2-(carboxymethoxy)ethoxy)pyridin-3-yl)-8-chloroquinazoline-4-carboxylic acid

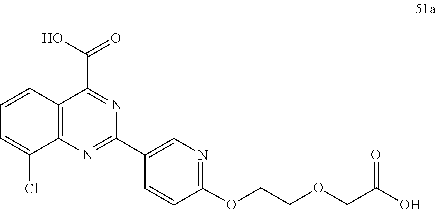 [SMILES: O=C(O)COCCOc1ccc(-c2nc(C(=O)O)c3cccc(Cl)c3n2)cn1]

Compound 51a was prepared in analogy to Example 50 by using ethyl 2-(2-hydroxyethoxy)acetate (185 mg, 1.25 mmol) instead of cis-methyl 3-(2-hydroxyethoxy)cyclobutanecarboxylate. 2-(6-(2-(Carboxymethoxy)ethoxy)pyridin-3-yl)-8-chloroquinazoline-4-carboxylic acid (compound 51a) was obtained as a white solid. MS obsd. (ESI⁺) [(M+H)⁺]: 404.0.

### Step 2: Preparation of 2-[2-[[5-(8-chloroquinazolin-2-yl)-2-pyridyl]oxy]ethoxy]acetic acid

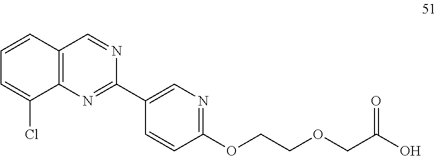 [SMILES: O=C(O)COCCOc1ccc(-c2ncc3cccc(Cl)c3n2)cn1]

A mixture of 2-(6-(2-(carboxymethoxy)ethoxy)pyridin-3-yl)-8-chloroquinazoline-4-carboxylic acid (compound 51a, 30 mg, 0.074 mmol) in NMP (2 mL) was charged with Argon, and the mixture was heated at 170° C. under microwave irradiation for 0.5 hr. The residue was purified by preparative HPLC to give 2-[2-[[5-(8-chloroquinazolin-2-yl)-2-pyridyl]oxy]ethoxy]acetic acid (Example 51, 17 mg, 62.3%) as a white solid. ¹H NMR (400 MHz, DMSO-d₆) δ ppm 9.77 (s, 1H), 9.33 (d, J=1.96 Hz, 1H), 8.73-8.83 (m, 1H), 8.13-8.26 (m, 2H), 7.66-7.77 (m, 1H), 7.02-7.11 (m, 1H), 4.46-4.56 (m, 2H), 4.10 (s, 2H), 3.82-3.91 (m, 2H). MS obsd. (ESI⁺) [(M+H)⁺]: 360.1.

### Example 52

### 8-Chloro-2-(6-chloro-3-pyridyl)-N-(2-hydroxyethyl)quinazoline-4-carboxamide

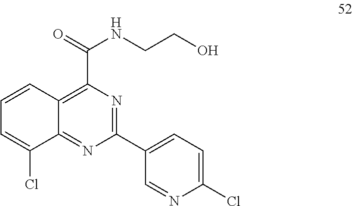 [SMILES: O=C(NCCO)c1nc(-c2ccc(Cl)nc2)nc2c(Cl)cccc12]

### Preparation of 8-chloro-2-(6-chloro-3-pyridyl)-N-(2-hydroxyethyl)quinazoline-4-carboxamide

Example 52 was prepared in analogy to Example 35 by using 8-chloro-2-(6-chloro-3-pyridyl)quinazoline-4-carboxylic acid (Example 45, 189 mg, 0.591 mmol) instead of 8-chloro-2-(4-methoxyphenyl)quinazoline-4-carboxylic acid (Example 33). 8-Chloro-2-(6-chloro-3-pyridyl)-N-(2-hydroxyethyl)quinazoline-4-carboxamide (Example 52, 200 mg) was obtained as an off-white solid (200 mg, 93.2%). ¹H NMR (400 MHz, DMSO-d₆) δ ppm 9.67 (d, J=2.45 Hz, 1H), 9.35 (br t, J=5.75 Hz, 1H), 9.01 (dd, J=8.44, 2.45 Hz, 1H), 8.94 (dd, J=8.56, 1.22 Hz, 1H), 8.31 (dd, J=7.58, 1.22 Hz, 1H), 7.74-7.85 (m, 2H), 4.87 (t, J=5.50 Hz, 1H), 3.63 (q, J=5.95 Hz, 2H), 3.44-3.54 (m, 2H). MS obsd. (ESI⁺) [(M+H)⁺]: 363.0.

### Example 53

### [8-Chloro-2-(6-chloro-3-pyridyl)quinazolin-4-yl]-morpholino-methanone

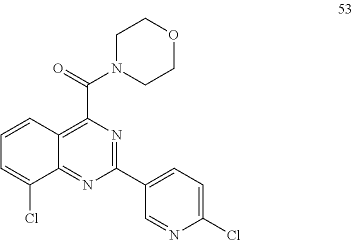 [SMILES: O=C(c1nc(-c2ccc(Cl)nc2)nc2c(Cl)cccc12)N1CCOCC1]

### Preparation of [8-chloro-2-(6-chloro-3-pyridyl)quinazolin-4-yl]-morpholino-methanone

Example 53 was prepared in analogy to Example 35 by using 8-chloro-2-(6-chloro-3-pyridyl)quinazoline-4-carboxylic acid (Example 45, 250 mg, 0.781 mmol) and morpholine (67.9 mg, 0.78 mmol) as the starting materials instead of 8-chloro-2-(4-methoxyphenyl)quinazoline-4-carboxylic acid (Example 33) and 2-aminoethanol. [8-Chloro-2-(6-chloro-3-pyridyl)quinazolin-4-yl]-morpholino-methanone (Example 53, 200 mg) was obtained as a white solid (200 mg, 71%). ¹H NMR (400 MHz, DMSO-d₆) δ ppm 9.46 (d, J=1.96 Hz, 1H), 8.86 (dd, J=8.44, 2.45 Hz, 1H), 8.32 (dd, J=7.58, 1.22 Hz, 1H), 8.06 (dd, J=8.25, 1.16 Hz, 1H), 7.72-7.82 (m, 2H), 3.82 (br d, J=3.91 Hz, 4H), 3.51 (t, J=4.77 Hz, 2H), 3.34 (m, 2H). MS obsd. (ESI⁺) [(M+H)⁺]: 389.0.

### Example 54

### cis-3-[2-[[5-(8-Chloro-4-hydroxy-quinazolin-2-yl)-2-pyridyl]oxy]ethoxy]cyclobutanecarboxylic acid

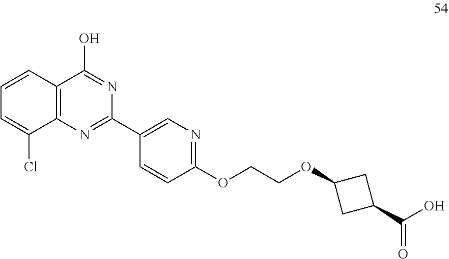 [SMILES: O=C(O)[C@H]1C[C@@H](OCCOc2ccc(-c3nc(O)c4cccc(Cl)c4n3)cn2)C1]

### Preparation of cis-3-[2-[[5-(8-chloro-4-hydroxy-quinazolin-2-yl)-2-pyridyl]oxy]ethoxy]cyclobutanecarboxylic acid

To a stirred mixture solution of cis-methyl 3-(2-hydroxyethoxy)cyclobutanecarboxylate (121 mg, 0.694 mmol) in THF (20 mL) was added sodium hydride (185 mg, 4.62 mmol) and stirred at rt for 0.5 hr. Then a solution of [8-chloro-2-(6-chloro-3-pyridyl)quinazolin-4-yl]-morpholino-methanone (Example 53, 180 mg, 0.462 mmol) in DMF (10 mL) was added to above reaction mixture, which was heated at 60° C. for 20 hrs, then neutralized with 1N aqueous HCl solution and partitioned between EtOAc (10 mL) and water (10 mL). The separated aqueous phase was extracted with EtOAc (10 mL) twice. The combined organic phase was dried over Na₂SO₄ and concentrated in vacuo. The residue was purified by preparative HPLC to give cis-3-[2-[[5-(8-chloro-4-hydroxy-quinazolin-2-yl)-2-pyridyl]oxy]ethoxy]cyclobutanecarboxylic acid (Example 54, 40 mg, 20.4%) as a white solid. ¹H NMR (400 MHz, DMSO-d₆) δ ppm 12.63-12.95 (m, 1H), 11.97-12.21 (m, 1H), 9.00 (d, J=2.45 Hz, 1H), 8.51 (dd, J=8.80, 2.57 Hz, 1H), 8.10 (dd, J=7.95, 1.34 Hz, 1H), 7.99 (dd, J=7.76, 1.41 Hz, 1H), 7.48 (t, J=7.89 Hz, 1H), 7.05 (d, J=8.80 Hz, 1H), 4.45 (dd, J=5.50, 3.67 Hz, 2H), 3.88-4.03 (m, 1H), 3.59-3.74 (m, 2H), 2.55-2.65 (m, 1H), 2.44 (m, 2H), 1.91-2.19 (m, 2H). MS obsd. (ESI⁺) [(M+H)⁺]: 416.2.

### Example 55

### 3-(2-(4-(8-Chloro-4-methoxyquinazolin-2-yl)phenoxy)ethoxy)cyclobutanecarboxylic acid

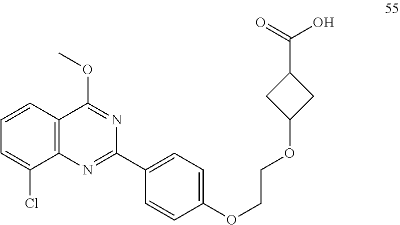 [SMILES: COc1nc(-c2ccc(OCCOC3CC(C(=O)O)C3)cc2)nc2c(Cl)cccc12]

### Step 1: Preparation of 2,8-dichloro-4-methoxyquinazoline

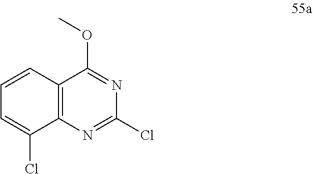

A mixture of 2,4,8-trichloroquinazoline (400 mg, 1.71 mmol) and K₂CO₃ (237 mg, 1.71 mmol) in MeOH (5 mL) was stirred at rt for 2 hrs. The solvent was removed in vacuo and the residue was partitioned between EtOAc (10 mL) and water (10 mL), the separated aqueous phase was extracted with EtOAc (10 mL) twice. The combined organic phase was dried over Na₂SO₄ and concentrated in vacuo to give the crude 2,8-dichloro-4-methoxyquinazoline (compound 55a, 380 mg, 96.8%) as a solid which was used in next step without further purification. MS obsd. (ESI⁺) [(M+H)⁺]: 229.0.

### Step 2: Preparation of 4-(8-chloro-4-methoxyquinazolin-2-yl)phenol

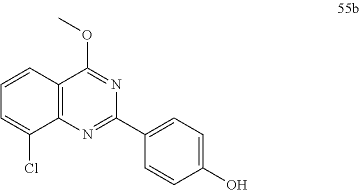 [SMILES: COc1nc(-c2ccc(O)cc2)nc2c(Cl)cccc12]

A mixture of 2,8-dichloro-4-methoxyquinazoline (compound 55a, 380 mg, 1.66 mmol), (4-hydroxyphenyl)boronic acid (206 mg, 1.49 mmol) and Pd(Ph₃P)₄ (192 mg, 0.166 mmol in 1,4-Dioxane (10 mL) and 2N K₂CO₃ (2 mL) was charged with nitrogen and heated at 110° C. for 2.5 hrs. After the reaction was completed, the mixture was adjusted to pH 4 with 1 N aqueous HCl, then partitioned between EtOAc (10 mL) and water (10 mL), and the separated aqueous phase was extracted with EtOAc (10 mL) twice. The combined organic phase was dried over Na₂SO₄ and concentrated in vacuo to give crude 4-(8-chloro-4-methoxyquinazolin-2-yl)phenol (compound 55b, 400 mg, 84.1%) as a solid which was used in next step without further purification. MS obsd. (ESI⁺) [(M+H)⁺]: 287.1.

### Step 3: Preparation of methyl 3-(2-(4-(8-chloro-4-methoxyquinazolin-2-yl)phenoxy)ethoxy)cyclobutanecarboxylate

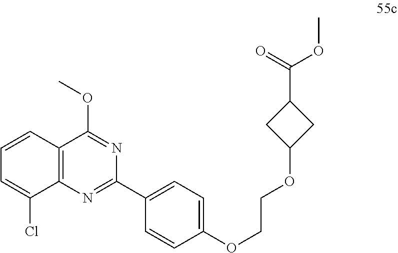 [SMILES: COC(=O)C1CC(OCCOc2ccc(-c3nc(OC)c4cccc(Cl)c4n3)cc2)C1]

A mixture of 4-(8-chloro-4-methoxyquinazolin-2-yl)phenol (compound 55b, 400 mg, 1.4 mmol), methyl 3-[2-(p-tolylsulfonyloxy)ethoxy]cyclobutanecarboxylate (compound 15d, 458 mg, 1.4 mmol) and K₂CO₃ (289 mg, 2.09 mmol) in DMF (4 mL) was stirred at 60° C. for 2 hrs. After the reaction was completed, the mixture was partitioned between EtOAc (10 mL) and water (10 mL), and the separated aqueous phase was extracted with EtOAc (10 mL) twice. The combined organic phase was dried over Na₂SO₄ and concentrated in vacuo to give the crude methyl 3-(2-(4-(8-chloro-4-methoxyquinazolin-2-yl)phenoxy)ethoxy)cyclobutanecarboxylate (compound 55c, 500 mg, 80.9%) as a solid which was used in next step without further purification. MS obsd. (ESI⁺) [(M+H)⁺]: 443.1.

### Step 4: Preparation of 3-(2-(4-(8-chloro-4-methoxyquinazolin-2-yl)phenoxy)ethoxy)cyclobutanecarboxylic acid

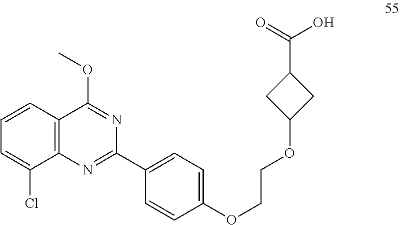 [SMILES: COc1nc(-c2ccc(OCCOC3CC(C(=O)O)C3)cc2)nc2c(Cl)cccc12]

To a solution of methyl 3-(2-(4-(8-chloro-4-methoxyquinazolin-2-yl)phenoxy)ethoxy)cyclobutanecarboxylate (compound 55c, 300 mg, 0.677 mmol) in THF (4 mL) was added 1N LiOH (2 mL) at rt, and then the mixture was heated at 50° C. for 1 hr. After the reaction was completed, the mixture was adjusted to pH 4 with 1 N aqueous HCl and partitioned between EtOAc (10 mL) and water (10 mL), and the separated aqueous phase was extracted with EtOAc (10 mL) twice. The combined organic phase was dried over Na₂SO₄ and concentrated in vacuo. The residual was purified by preparative HPLC to give 3-(2-(4-(8-chloro-4-methoxyquinazolin-2-yl)phenoxy)ethoxy)cyclobutanecarboxylic acid (Example 55, 180 mg, 60.7%) as a solid. ¹H NMR (400 MHz, DMSO-d6) δ ppm 8.51 (d, J=8.80 Hz, 2H), 8.07 (d, J=7.83 Hz, 2H), 7.54 (t, J=7.83 Hz, 1H), 7.13 (d, J=9.05 Hz, 2H), 4.26 (s, 3H), 4.15-4.20 (m, 2H), 3.96 (s, 1H), 3.66-3.72 (m, 2H), 2.55-2.67 (m, 1H), 2.37-2.49 (m, 2H), 1.96-2.23 (m, 2H). MS obsd. (ESI⁺) [(M+H)⁺]: 429.1.

### Example 56

### 3-[2-[4-[8-chloro-4-(trifluoromethyl)quinazolin-2-yl]phenoxy]ethoxy]cyclobutanecarboxylic acid

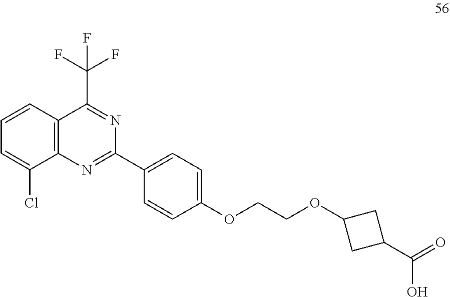 [SMILES: O=C(O)C1CC(OCCOc2ccc(-c3nc(C(F)(F)F)c4cccc(Cl)c4n3)cc2)C1]

### Step 1: Preparation of methyl 3-chloro-2-[(4-methoxybenzoyl)amino]benzoate

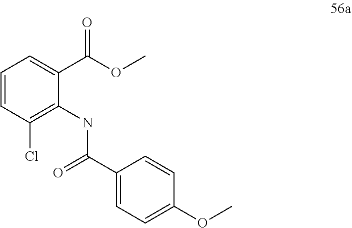 [SMILES: COC(=O)c1cccc(Cl)c1NC(=O)c1ccc(OC)cc1]

To a solution of methyl 2-amino-3-chloro-benzoate (0.98 g, 5.28 mmol) in THF (5 mL), pyridine (2.0 mL, 24.73 mmol) was added 4-methoxybenzoyl chloride (1.0 g, 5.86 mmol), and then the mixture was heated at 60° C. for 12 hrs. After the reaction was completed, the reaction mixture was washed by 1 N aqueous HCl (20 mL) and extracted with EtOAc (50 mL) twice. The combined organic phase was dried over Na₂SO₄ and concentrated in vacuo. The residue was purified by column chromatography on silica gel using gradient elution with PE-EtOAc from 100:0 to 100:30 (v/v) to give methyl 3-chloro-2-[(4-methoxybenzoyl)amino]benzoate (compound 56a, 600 mg, 32.01%) as white solid. MS obsd. (ESI⁺) [(M+H)⁺]: 320.1.

### Step 2: Preparation of 3-chloro-2-[(4-methoxybenzoyl)amino]benzoic acid

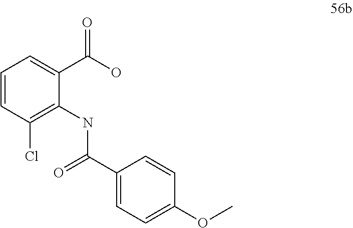 [SMILES: COc1ccc(C(=O)Nc2c(Cl)cccc2C(=O)O)cc1]

To a solution of methyl 3-chloro-2-[(4-methoxybenzoyl)amino]benzoate (compound 56a, 600 mg, 1.88 mmol) in MeOH (10 mL), water (2 mL) was added sodium hydroxide (750 mg, 18.77 mmol), and the solution was stirred at 25° C. for 2 hrs. After the reaction was completed, the reaction mixture was adjusted to pH4 by 1N aqueous HCl solution and extracted with EtOAc (50 mL) twice. The combined organic phase was dried over Na₂SO₄ and concentrated in vacuo to give 3-chloro-2-[(4-methoxybenzoyl)amino]benzoic acid (compound 56b, 500 mg, 87.16%) as a white solid. MS obsd. (ESI⁺) [(M+H)⁺]: 306.0.

### Step 3: Preparation of 8-chloro-2-(4-methoxyphenyl)-3, 1-benzoxazin-4-one

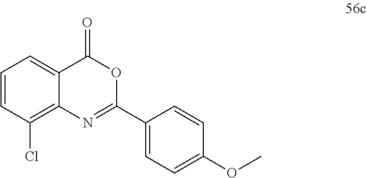 [SMILES: COc1ccc(-c2nc3c(Cl)cccc3c(=O)o2)cc1]

A solution of 3-chloro-2-[(4-methoxybenzoyl)amino]benzoic acid (compound 56b, 500 mg, 1.64 mmol) in sulfoxide chloride (2.0 g, 16.4 mmol) was stirred at 80° C. for 12 hrs. After being cooled to rt, the reaction mixture was concentrated in vacuo to give the crude 8-chloro-2-(4-methoxyphenyl)-3, 1-benzoxazin-4-one (compound 56c, 500 mg) as light yellow solid. MS obsd. (ESI⁺) [(M+H)⁺]: 288.1.

### Step 4: Preparation of N-[2-chloro-6-(2,2,2-trifluoroacetyl)phenyl]-4-methoxy-benzamide

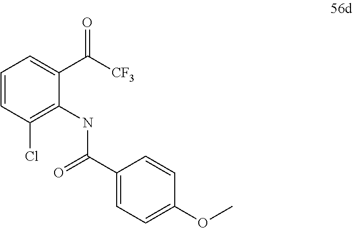 [SMILES: COc1ccc(C(=O)Nc2c(Cl)cccc2C(=O)C(F)(F)F)cc1]

To a solution of 8-chloro-2-(4-methoxyphenyl)-3, 1-benzoxazin-4-one (compound 56c, 500 mg, 1.74 mmol) in DMSO (10 mL) was added trifluoromethyl trimethylsilane (370 mg, 2.61 mmol), and TBAF in THF (0.35 mL, 0.350 mmol) at rt. The mixture was stirred for 5 hrs, then quenched by water (20 mL) and extracted with EtOAc (50 mL) twice. The combined organic phase was dried over Na₂SO₄ and concentrated in vacuo. The residue was purified by column chromatography on silica gel using gradient elution with PE-EtOAc from 100:0 to 100:30 (v/v) to give N-[2-chloro-6-(2,2,2-trifluoroacetyl)phenyl]-4-methoxy-benzamide (compound 56d, 150 mg, 24.13%) as a yellow solid. MS obsd. (ESI⁺) [(M+H)⁺]: 324.1.

### Step 5: Preparation of 8-chloro-2-(4-methoxyphenyl)-4-(trifluoromethyl)quinazoline

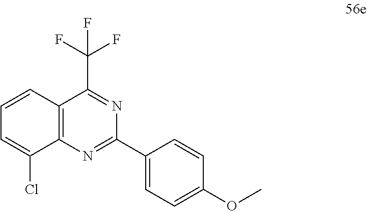 [SMILES: COc1ccc(-c2nc(C(F)(F)F)c3cccc(Cl)c3n2)cc1]

To a solution of N-[2-chloro-6-(2,2,2-trifluoroacetyl)phenyl]-4-methoxy-benzamide (compound 56d, 2.0 g, 5.59 mmol) in DMSO (15 mL) was added ammonium acetate (1.29 g, 16.77 mmol), and then the mixture was heated to 100° C. for 12 hrs. After the reaction was completed, the reaction mixture was quenched by water (20 mL) and extracted with EtOAc (50 mL) twice. The combined organic phase was dried over Na₂SO₄ and concentrated in vacuo. The residue was purified by column chromatography on silica gel using gradient elution with PE-EtOAc from 100:0 to 100:30 (v/v) to give 8-chloro-2-(4-methoxyphenyl)-4-(trifluoromethyl)quinazoline (compound 56e, 1.8 g, 95.05%) as a colorless oil. MS obsd. (ESI⁺) [(M+H)⁺]: 339.1.

### Step 6: Preparation of 4-[8-chloro-4-(trifluoromethyl)quinazolin-2-yl]phenol

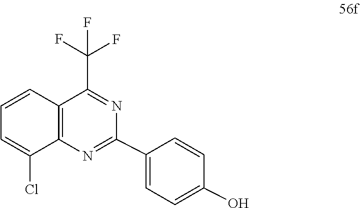 [SMILES: Oc1ccc(-c2nc(C(F)(F)F)c3cccc(Cl)c3n2)cc1]

To a solution of 8-chloro-2-(4-methoxyphenyl)-4-(trifluoromethyl)quinazoline (compound 56e, 500 mg, 1.48 mmol) in DCM (15 mL) at 0° C. was added boron tribromide (0.3 mL, 1.48 mmol), and the mixture was stirred at 0° C. for 1 hr. After the reaction was completed, the reaction mixture was quenched by 1% aqueous Na₂CO₃ solution (20 mL) carefully and extracted with EtOAc (50 mL) twice. The combined organic phase was dried over Na₂SO₄ and concentrated in vacuo to give the crude 4-[8-chloro-4-(trifluoromethyl)quinazolin-2-yl]phenol (compound 56f, 600 mg) as orange solid. MS obsd. (ESI⁺) [(M+H)⁺]: 325.2.

### Step 7: Preparation of 3-[2-[4-[8-chloro-4-(trifluoromethyl)quinazolin-2-yl]phenoxy]ethoxy]cyclobutanecarboxylic acid

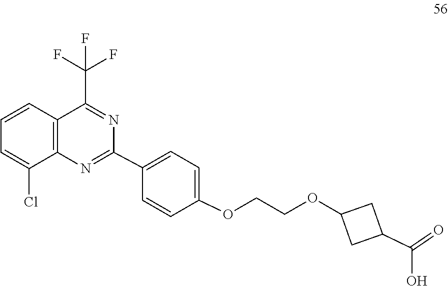 [SMILES: O=C(O)C1CC(OCCOc2ccc(-c3nc(C(F)(F)F)c4cccc(Cl)c4n3)cc2)C1]

To a solution of 4-[8-chloro-4-(trifluoromethyl)quinazolin-2-yl]phenol (compound 56f, 300 mg, 0.920 mmol) in DMF (5 mL) was added methyl 3-[2-(p-tolylsulfonyloxy)ethoxy]cyclobutanecarboxylate (compound 15d, 152 mg, 0.460 mmol) and potassium carbonate (383 mg, 2.77 mmol). The reaction mixture was heated at 70° C. for 6 hrs. After being cooled to rt, 1N aqueous LiOH (2.0 mL, 2 mmol) was added to above reaction mixture and stirred for another 2 hrs. After the reaction was completed, the reaction mixture was adjusted by 0.5 N aqueous HCl to pH4 and extracted with EtOAc (50 mL) twice. The combined organic phase was dried over Na₂SO₄ and concentrated in vacuo. The residue was purified by preparative HPLC to give 3-[2-[4-[8-chloro-4-(trifluoromethyl)quinazolin-2-yl]phenoxy]ethoxy]cyclobutanecarboxylic acid (Example 56, 15 mg, 3.48%) as a white solid. ¹H NMR (DMSO-d₆, 400 MHz): δ ppm 12.19 (br s, 1H), 8.53 (d, J=8.9 Hz, 2H), 8.34 (dd, J=7.6, 0.7 Hz, 1H), 8.18 (br d, J=8.3 Hz, 1H), 7.74-7.88 (m, 1H), 7.20 (d, J=8.9 Hz, 2H), 4.12-4.34 (m, 2H), 3.96 (br quin, J=7.3 Hz, 1H), 3.60-3.76 (m, 2H), 2.91 (br d, J=3.9 Hz, 1H), 2.35-2.47 (m, 2H), 2.11-2.24 (m, 1H), 1.95-2.05 ppm (m, 1H). MS obsd. (ESI⁺) [(M+H)⁺]: 467.

### BIOLOGICAL EXAMPLES

### Example 57: Engineered HepDES19 Primary Screen Assay

The assay was employed to screen for novel HBV cccDNA inhibitors. HepDES19 is a HBV cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res. (2006), 72(2), 116-124; Guo, H. et al., J. Virol. (2007), 81(22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per mL. For compound testing, the frozen cells were thawed and seeded into 96-well plates at a density of 6×10⁴ cells per well. At 24 hrs after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (Donjindo, Cat. CK04-20). IC₅₀ values were derived from the dose-response curve using 4 parameter logistic curve fit method.

The compounds of the present invention were tested for their capacity to inhibit extracellular HBeAg level as described herein. The compounds of this invention were found to have IC₅₀ below 30 μM. Particular compounds of formula (I) were found to have IC₅₀ below 1.0 μM. Results of HepDES19 primary screen assay are given in Table 1.

TABLE 1
Activity data in HepDES19 primary screen assay

| Example No. | IC₅₀ (μM) |
|---|---|
| 1 | 0.10 |
| 2 | 0.36 |
| 3 | 0.75 |
| 4 | 3.62 |
| 5 | 4.35 |
| 6 | 6.16 |
| 7 | 0.22 |
| 8 | 0.46 |
| 9 | 1.93 |
| 10 | 1.04 |
| 11 | 4.49 |
| 12 | 0.063 |
| 13 | 1.03 |
| 14 | 29.9 |
| 15 | 0.060 |
| 16 | 2.39 |
| 17 | 4.66 |
| 18 | 1.93 |
| 19 | 3.50 |
| 20 | 3.66 |
| 21 | 0.094 |
| 22 | 7.13 |
| 23 | 0.22 |
| 24 | 0.094 |
| 25 | 4.68 |
| 26 | 0.091 |
| 27 | 0.59 |
| 28 | 0.30 |
| 29 | 1.23 |
| 30 | 7.63 |
| 31 | 17.8 |
| 32 | 6.19 |
| 33 | 7.75 |
| 34 | 2.22 |
| 35 | 16.1 |
| 36 | 0.18 |
| 37 | 2.24 |
| 38 | 1.16 |
| 39 | 1.17 |
| 40 | 0.082 |
| 42 | 16.9 |
| 43 | 12.9 |
| 44 | 8.70 |
| 45 | 0.66 |
| 46 | 0.11 |
| 48 | 7.57 |
| 49 | 11 |
| 50 | 15 |
| 51 | 4.86 |
| 52 | 0.16 |
| 53 | 0.083 |
| 54 | 3.42 |
| 55 | 0.026 |
| 56 | 7.86 |

1. A compound of the formula (I), 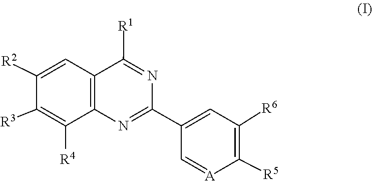
wherein
A is CH or N;
R¹ is H, (C₁₋₆alkyl)₂aminoC₁₋₆alkyl, C₁₋₆alkoxy, C₁₋₆alkoxyC₁₋₆alkyl, C₁₋₆alkoxycarbonyl, C₁₋₆alkyl, C₁₋₆alkyl-C(O)O—C₁₋₆alkyl-, carbamoyl, carboxy, haloC₁₋₆alkyl, haloC₁₋₆alkylaminocarbonyl, hydroxy, hydroxyC₁₋₆alkyl, hydroxyC₁₋₆alkylaminocarbonyl, morpholinylC₁₋₆alkyl or morpholinylcarbonyl;
R² is H or halogen;
R³ is H, halogen or haloC₁₋₆alkyl;
R⁴ is H or halogen;
R⁵ is H, (C₁₋₆alkoxycarbonyl)phenylC₁₋₆alkoxy, (C₁₋₆alkoxycarbonyl)piperidinyl, (C₁₋₆alkoxycarbonylC₁₋₆alkoxy)C₁₋₆alkoxy, (C₁₋₆alkyl)₂amino, (carboxyC₁₋₆alkoxy)C₁₋₆alkoxy, (carboxyC₃₋₇cycloalkoxy)C₁₋₆alkoxy, (hydroxyC₁₋₆alkyl)₂amino, C₁₋₆alkoxy, carboxyphenylC₁₋₆alkoxy, carboxypiperidinyl, halogen, hydroxy, hydroxyC₁₋₆alkoxy, morpholinyl or pyrrolidinyl;
R⁶ is H, halogen, hydroxyC₁₋₆alkyl or haloC₁₋₆alkyl;
with the proviso that R⁵ and R⁶ are not H simultaneously;
or pharmaceutically acceptable salt, or enantiomer, or diastereomer thereof.

2. A compound according to claim 1, wherein
A is CH or N;
R¹ is H, acetoxymethyl, carbamoyl, carboxy, chloroethylaminocarbonyl, dimethylaminomethyl, ethoxycarbonyl, hydroxy, hydroxyethylaminocarbonyl, hydroxymethyl, methoxy, methoxycarbonyl, methoxymethyl, methyl, morpholinylcarbonyl, morpholinylmethyl or trifluoromethyl;
R² is H or chloro;
R³ is H, bromo or trifluoromethyl;
R⁴ is H or chloro;
R⁵ is H, (carboxycyclobutoxy)ethoxy, (carboxymethoxy)ethoxy, (hydroxyethyl)₂amino, (methoxycarbonyl)phenylmethoxy, (methoxycarbonyl)piperidinyl, (methoxyoxoethoxy)ethoxy, bromo, carboxyphenylmethoxy, carboxypiperidinyl, chloro, dimethylamino, ethoxy, hydroxy, hydroxyethoxy, methoxy, morpholinyl, pyrrolidinyl;
R⁶ is H, bromo, hydroxymethyl or trifluoromethyl;
with the proviso that R⁵ and R⁶ are not H simultaneously;
or pharmaceutically acceptable salt, or enantiomer, or diastereomer thereof.

3. A compound according to claim 1 or 2, wherein R¹ is H, carbamoyl, carboxy, hydroxy, hydroxyC₁₋₆alkylaminocarbonyl, hydroxyC₁₋₆alkyl, C₁₋₆alkyl or morpholinylcarbonyl.

4. A compound according to claim 3, wherein R¹ is H, carbamoyl, carboxy, hydroxy, hydroxyethylaminocarbonyl, hydroxymethyl, methyl or morpholinylcarbonyl.

5. A compound according to claim 4, wherein R² is H.

6. A compound according to claim 5, wherein R⁵ is H, (C₁₋₆alkoxycarbonyl)piperidinyl, (C₁₋₆alkoxycarbonylC₁₋₆alkoxy)C₁₋₆alkoxy, (C₁₋₆alkyl)₂amino, (carboxyC₁₋₆alkoxy)C₁₋₆alkoxy, (carboxyC₃₋₇cycloalkoxy)C₁₋₆alkoxy, (hydroxyC₁₋₆alkyl)₂amino, C₁₋₆alkoxy, carboxyphenylC₁₋₆alkoxy, halogen, hydroxyC₁₋₆alkoxy or morpholinyl.

7. A compound according to claim 6, wherein R⁵ is H, (carboxycyclobutoxy)ethoxy, (carboxymethoxy)ethoxy, (hydroxyethyl)₂amino, (methoxycarbonyl)piperidinyl, (methoxyoxoethoxy)ethoxy, bromo, carboxyphenylmethoxy chloro, dimethylamino, hydroxyethoxy, methoxy or morpholinyl.

8. A compound according to claim 1 or 2, selected from
2-(4-Bromophenyl)-8-chloroquinazoline;
2-(3-Bromophenyl)-8-chloroquinazoline;
7-Bromo-4-methyl-2-[3-(trifluoromethyl)phenyl]quinazoline;
2-(3-Bromophenyl)-6-chloro-4-methyl-7-(trifluoromethyl)quinazoline;
4-(8-Chloroquinazolin-2-yl)phenol;
Methyl 3-((4-(8-chloroquinazolin-2-yl)phenoxy)methyl)benzoate;
3-((4-(8-Chloroquinazolin-2-yl)phenoxy)methyl)benzoic acid;
3-((4-(8-Chloro-4-methylquinazolin-2-yl)phenoxy)methyl)benzoic acid;
2-[4-(8-Chloro-4-methyl-quinazolin-2-yl)phenoxy]ethanol;
Methyl 2-[2-[4-(8-chloro-4-methyl-quinazolin-2-yl)phenoxy]ethoxy]acetate;
2-[2-[4-(8-Chloro-4-methyl-quinazolin-2-yl)phenoxy]ethoxy]acetic acid;
cis-3-[2-[4-(8-Chloro-4-methyl-quinazolin-2-yl)phenoxy]ethoxy]cyclobutanecarboxylic acid;
8-Chloro-2-(4-chlorophenyl)-4-methylquinazoline;
2-(2-(4-(4-Methyl-7-(trifluoromethyl)quinazolin-2-yl)phenoxy)ethoxy)acetic acid;
3-(2-(4-(4-Methyl-7-(trifluoromethyl)quinazolin-2-yl)phenoxy)ethoxy) cyclobutanecarboxylic acid;
2-(3-Bromophenyl)-4-(methoxymethyl)-7-(trifluoromethyl)quinazoline;
1-(2-(3-Bromophenyl)-7-(trifluoromethyl)quinazolin-4-yl)-N,N-dimethylmethanamine;
4-((2-(3-Bromophenyl)-7-(trifluoromethyl)quinazolin-4-yl)methyl)morpholine;
2-(2-(3-Bromobenzamido)-4-(trifluoromethyl)phenyl)-2-oxoethyl acetate;
(2-(3-Bromophenyl)-7-(trifluoromethyl)quinazolin-4-yl)methanol;
(8-Chloro-2-(4-chlorophenyl)quinazolin-4-yl)methanol;
(3-(4-Methyl-7-(trifluoromethyl)quinazolin-2-yl)phenyl)methanol;
8-Chloro-2-(6-chloropyridin-3-yl)-4-methylquinazoline;
8-Chloro-2-(6-chloropyridin-3-yl)quinazoline;
5-(8-Chloroquinazolin-2-yl)pyridin-2-ol;
5-(8-Chloroquinazolin-2-yl)-N,N-dimethylpyridin-2-amine;
4-(5-(8-Chloroquinazolin-2-yl)pyridin-2-yl)morpholine;
2,2′-((5-(8-Chloroquinazolin-2-yl)pyridin-2-yl)azanediyl)diethanol;
Methyl 1-(5-(8-chloroquinazolin-2-yl)pyridin-2-yl)piperidine-4-carboxylate;
1-(5-(8-chloroquinazolin-2-yl)pyridin-2-yl)piperidine-4-carboxylic acid;
2-(3-Bromo-phenyl)-7-trifluoromethyl-quinazoline-4-carboxylic acid;
Methyl 2-(3-bromophenyl)-7-(trifluoromethyl)quinazoline-4-carboxylate;
8-Chloro-2-(4-methoxyphenyl)quinazoline-4-carboxylic acid;
8-chloro-2-(4-methoxyphenyl)quinazoline-4-carboxamide;
8-Chloro-N-(2-hydroxyethyl)-2-(4-methoxyphenyl)quinazoline-4-carboxamide;
8-Chloro-2-(4-methoxyphenyl)quinazoline;
2-(4-Bromophenyl)-8-chloroquinazoline-4-carboxylic acid;
ethyl 2-(4-bromophenyl)-8-chloro-quinazoline-4-carboxylate;
2-(4-Bromophenyl)-8-chloro-N-(2-chloroethyl)quinazoline-4-carboxamide;
2-(4-Bromophenyl)-8-chloro-4-methyl-quinazoline;
2-[4-(8-Chloroquinazolin-2-yl)phenoxy]ethanol;
cis-3-[2-[4-(8-Chloroquinazolin-2-yl)phenoxy]ethoxy]cyclobutanecarboxylic acid;
2-(4-Bromophenyl)-7-(trifluoromethyl)quinazoline-4-carboxylic acid;
[2-(4-Bromophenyl)-7-(trifluoromethyl)quinazolin-4-yl]-morpholino-methanone;
8-Chloro-2-(6-chloro-3-pyridyl)quinazoline-4-carboxylic acid;
8-chloro-2-(6-chloro-3-pyridyl)quinazoline-4-carboxamide;
8-Chloro-2-(6-methoxy-3-pyridyl)quinazoline-4-carboxylic acid;
8-Chloro-2-(6-ethoxy-3-pyridyl)quinazoline-4-carboxylic acid;
8-Chloro-2-(6-pyrrolidin-1-yl-3-pyridyl)quinazoline-4-carboxylic acid;
cis-2-[6-[2-(3-Carboxycyclobutoxy)ethoxy]-3-pyridyl]-8-chloro-quinazoline-4-carboxylic acid;
2-[2-[[5-(8-Chloroquinazolin-2-yl)-2-pyridyl]oxy]ethoxy]acetic acid;
8-Chloro-2-(6-chloro-3-pyridyl)-N-(2-hydroxyethyl)quinazoline-4-carboxamide;
[8-Chloro-2-(6-chloro-3-pyridyl)quinazolin-4-yl]-morpholino-methanone;
cis-3-[2-[[5-(8-Chloro-4-hydroxy-quinazolin-2-yl)-2-pyridyl]oxy]ethoxy]cyclobutanecarboxylic acid;
3-(2-(4-(8-Chloro-4-methoxyquinazolin-2-yl)phenoxy)ethoxy)cyclobutanecarboxylic acid; and
3-[2-[4-[8-chloro-4-(trifluoromethyl)quinazolin-2-yl]phenoxy]ethoxy]cyclobutanecarboxylic acid;
or pharmaceutically acceptable salt, or enantiomer, or diastereomer thereof.

9. A process for the preparation of a compound according to any one of claims 1 to 8 comprising any one of the following steps:
(a) intramolecular cyclization of compounds of formula (VII), 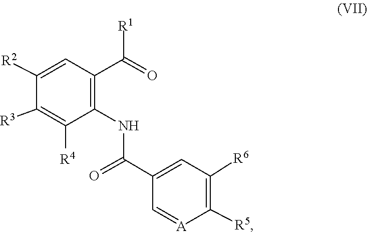
in the presence of an ammonium salt;
(b) cyclization of compounds of formula (V), 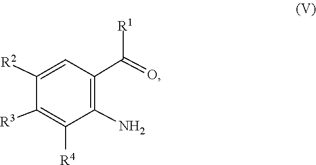
and arylamidine (VIII), 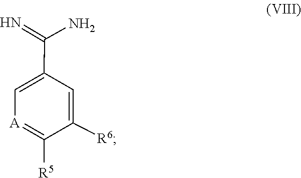
(c) substitution of compounds of formula (II), 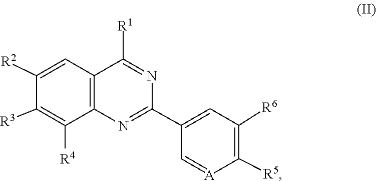
with HO-G₁ in the presence of a base, when R⁵ is X;
(d) substitution of compounds of formula (II), 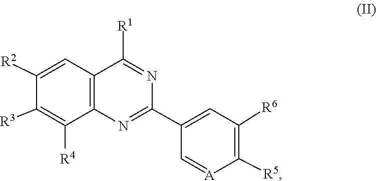
with amine (XXIII), when R⁵ is X;
(e) substitution of compounds of formula (II), 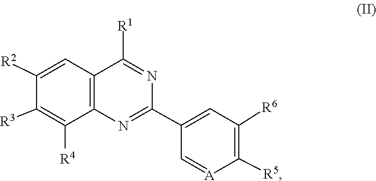
with Q-G₁ in the presence of a base, when R⁵ is OH;
(f) intramolecular cyclization of compounds of formula (XI), 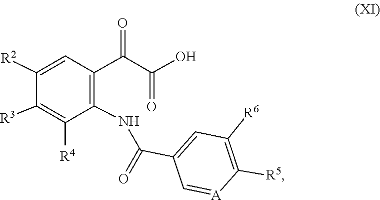
in the presence of an ammonium salt;
(g) ring rearrangement reaction of compounds of formula (XII), 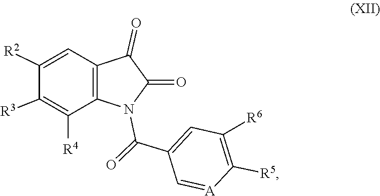
in the presence of an ammonium salt;
(h) coupling reaction of compounds of formula (XIII), 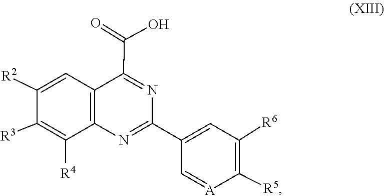
with amines (XXIV) in the presence of coupling reagents;
(i) decarboxylation of a compounds of formula (XIII), 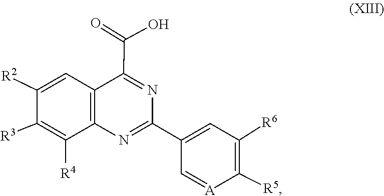
in the presence of decarboxylation reagents;
(j) Suzuki crossing coupling reaction of intermediate (XVI), 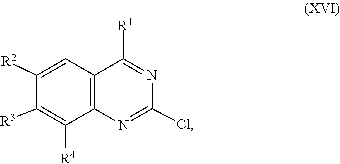
with aryl boric acid (XVII) or borate ester (XVIII) in the presence of a catalyst;
(k) substitution reaction of compounds of formula (XXI), 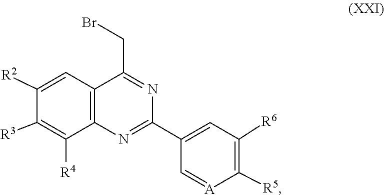
with amines or hydrolysis of compounds of formula (XXI) with a base;
wherein R¹, R², R³, R⁴, R⁵, R⁶ and A are defined as any one of claims 1 to 7.

10. A compound according to any one of claims 1 to 8 for use as therapeutically active substance.

11. A pharmaceutical composition comprising a compound in accordance with any one of claims 1 to 8 and a therapeutically inert carrier.

12. The use of a compound according to any one of claims 1 to 8 for the treatment or prophylaxis of HBV infection.

13. The use of a compound according to any one of claims 1 to 8 for the preparation of a medicament for the treatment or prophylaxis of HBV infection.

14. The use of a compound according to any one of claims 1 to 8 for the inhibition of HBV cccDNA.

15. The use of a compound according to any one of claims 1 to 8 for the inhibition of HBeAg.

16. The use of a compound according to any one of claims 1 to 8 for the inhibition of HBsAg.

17. The use of a compound according to any one of claims 1 to 8 for the inhibition of HBV DNA.

18. A compound according to any one of claims 1 to 8 for the treatment or prophylaxis of HBV infection.

19. A compound according to any one of claims 1 to 8, when manufactured according to a process of claim 9.

20. A method for the treatment or prophylaxis of HBV infection, which method comprises administering an effective amount of a compound as defined in any one of claims 1 to 8.
